(19) United States
(12) Patent Application Publication
(10) Pub. No.: US 2017/0143778 A1
(43) Pub. Date: May 25, 2017

(54) A METHOD OF FEEDING AN ANIMAL TO IMPROVE GROWTH EFFICIENCY

(71) Applicant: Micreos B.V., Wageningen (NL)

(72) Inventors: Johan Frans Willem Nuboer, Vught (NL); Steven Hagens, Bennekom (NL); Martin Johannes Loessner, Ebmatingen (CH); Mark Leonard Offerhaus, Den Haag (NL)

(73) Assignee: Micreos B.V., Wageningen (NL)

(21) Appl. No.: 15/321,053

(22) PCT Filed: Jun. 24, 2015
(86) PCT No.: PCT/EP2015/064248

§ 371 Date: Dec. 21, 2016

(30) Foreign Application Priority Data
Jun. 24, 2014 (EP) 14173673.6

(51) Int. Cl. A61K 35/76; A61K 9/00; C12N 7/00; A23K 50/10; A23K 20/147

(52) U.S. Cl. CPC A61K 35/76; A23K 50/10; A23K 20/147; C12N 7/00; A61K 9/0056; C12N 2795/10132

(57) ABSTRACT

The present invention relates to a method of feeding an animal to improve growth efficiency.

### FIELD OF THE INVENTION

The present invention relates to a method of feeding an animal to improve growth efficiency.

### BACKGROUND OF THE INVENTION

In livestock such as cattle, especially beef and dairy cattle, it is important to the industry to have a healthy economic output; margins are under pressure. One aspect that can improve economic output is to increase growth efficiency. The feed of livestock has already been improved for most efficient turnover. Accordingly, there is an urgent need for further means to improve growth efficiency of livestock.

### DESCRIPTION OF THE INVENTION

Surprisingly, it has now been demonstrated that the use of a bacteriophage in the feed of cattle significantly enhances growth efficiency.

Accordingly, in a first aspect the present invention provides a method of feeding an animal to improve growth efficiency, comprising:
providing the animal with a regular feed regime conducive to the growth of the animal, and
administering to the animal a source of a bacteriophage,
   wherein the source of a bacteriophage is administered to the animal in an effective dosage to improve growth efficiency.

Said method is herein referred to as a method according to the present invention.

A method of feeding is herein defined as any method that comprises administration of a feed or feedstuff or a composition comprising a feed or feedstuff to an animal and is not limited to any specific diet.

A feed is herein defined as any feed, feedstuff or composition comprising a feed or feedstuff suitable for an animal, such as, but not limited to corn, grass, barley, soy, crops, crop residues, pastures, hay, straw, silage, oils, milk, (liquid) milk replacer, carcass meal, fish meal, sprouted grains and legumes. For new-born and/or juvenile animals, the feed preferably is milk or a (liquid) milk replacer. A milk replacer is herein preferably a powdered milk substitute that is dissolved in water.

The feed or feedstuff may additionally comprise veterinary drugs, growth hormones, feed additives, and/or nutraceuticals to increase the production yield. The feed or feedstuff may be in any form known to the person skilled in the art, such as but not limited to liquid, pelleted or compressed feed.

A feed regime is herein defined as the administration of or making available of a feed or feedstuff to an animal on a regular basis such as that the animal is provided with enough feed or feedstuff to be able to grow or at least maintain homeostasis.

A source of a bacteriophage is herein defined as any composition that comprises a bacteriophage. In the context of the present invention, the source of the bacteriophage may also be depicted as a feed additive. The source may be a composition comprising isolated, purified bacteriophages, but the source may also be a non-purified composition comprising bacteriophages and/or parts thereof. The composition may e.g. comprise residues from the production of the bacteriophages such as medium components and host cells or parts thereof.

An effective dosage is herein defined as a dose that confers a statistically relevant improvement in growth or growth efficiency. A dosage is herein preferably expressed in absolute numbers of plaque forming units (pfu) as defined herein below.

Growth efficiency is herein defined as a measurable ratio, quantitatively determined as the ratio of output (such as increase in bodyweight) to input (such as kg of feed administered in a specific timeframe). An increase in growth efficiency can e.g. be depicted as a percentage; in this case the increase in bodyweight of an animal or a group of animals fed by the method according to the present invention during a specified timeframe is compared to the increase in bodyweight of an animal or a group of animals fed by an identical feed regime lacking the source of a bacteriophage according to the present invention.

In a method according to the invention, the source of the bacteriophage may be administered at least once during the treatment period. The treatment period may be a single day up to the entire life of the animal. Preferably, in a method according to the present invention, the source of a bacteriophage is administered to the animal at least once, twice, three times, 4, 5, 6, 7, 8, 9, 10, 11, 12, 13, 14, 15, 20, 25, 30, 40, 45, 50, 55, 60, 65, 70, 75, 80, 85, 90, 95, 100, 110, 120, 130, 140, 150, 200, 250, 300, 350, 400, 450, 500, 550 or at least 600 times within a period of at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, or at least 350 days up to the end of life of the animal, wherein the source of the bacteriophage is preferably administered at least once or at least twice a day. More preferably, the source of the bacteriophage is administered twice a day for approximately ten days.

Preferably, in a method according to the present invention, the animal is from the even-toed ungulate family Bovidae, more preferably from the subfamily Bovinae, even more preferably from the genus Bos, even more preferably from the species Bos taurus (domestic cattle), even more preferably from the group of dairy cattle or beef cattle.

Preferably, in a method according to the present invention, the animal is a new-born or a juvenile animal. A new-born animal is preferably within the age-range of one day and three months of age, more preferably within the age-range of one week and three months of age, even more preferably within the age-range of two weeks and three months of age. A juvenile animal is preferably within the age-range of one month and two years of age, more preferably within the age-range of one month and one year of age, even more preferably within the age-range of two months and six months of age.

The source of a bacteriophage is herein referred to as a source of a bacteriophage according to the present invention and may comprise or provide one or more different types of bacteriophages. Such bacteriophage is herein referred to as a bacteriophage according to the present invention. Preferably, at least one type of bacteriophage is effective against Salmonella. However, the source of a bacteriophage according to the invention may additionally comprise or provide one or more types of bacteriophages that are effective against another bacterium than Salmonella. The source of the bacteriophage according to the invention may, as said, comprise or provide one or more different types of bacteriophages that are effective against Salmonella. Preferably, a bacteriophage effective against Salmonella is effective against all Salmonella species; more preferably against all Salmonella enterica. Further preferred is a bacteriophage according to the present invention having a broad host range, preferably being able to infect and lyse at least 70, 80, 85, 90, 95 or 100% of all strains of Salmonella belonging to the group consisting of Salmonella enterica spp enterica serovars; more preferably belonging to the group consisting of serovars Infantis, Kentucky, Newport, Stanley, Hadar, Virchow, Typhimurium, Enteritidis, Agona, Anatum, Senftenberg, Montevideo, Muenster, Javiana, Heidelberg, Derby, Wien, Porci, Braederup, Panama, Panama, Newington, Livingston, Bredeney, Dublin, Cholerasuis, Give, Amherstiana, Salmone, Tennesee, Blockley, Indiana and Java. Within the context of the present invention, a broad host range is meant that at least 70% of the different Salmonella strains identified here above can be infected by a bacteriophage of the present invention. Even more preferred is a bacteriophage according to the present invention wherein said bacteriophage is able to infect and lyse at least 70, 75, 80, 85, 90, 95 or 100% of S. enterica strains. Preferably, at least one bacteriophage according to the present invention is able to infect and lyse Salmonella Re-LPS mutant (deep rough) strains, where only the inner core 2-keto-deoxy-d-octanoate (KDO) residues are present. This enables such bacteriophage according to the present invention, in contrast to e.g. bacteriophage Felix O1 which requires the terminal N-acetylglucosamine residue of the outer LPS core for infection (Lindberg, 1967; Lindberg and Holme, 1969), to also infect deep rough strains. Preferable, a bacteriophage according to the present invention is able to infect and lyse Salmonella LPS synthesis knock-out strains. Infection and lysis of a given bacterial strain with a bacteriophage according to the present invention can be quantitatively tested by any suitable assay known by the person skilled in the art. In a preferred assay, infection and subsequent lysis is tested by spot-on-the-lawn method. In brief, Dry LB agar plates are flooded with 4 mL of log-phase culture of a bacterial strain to be tested, excess culture is removed and the agar plates are dried for 30 minutes (30° C.). 3 μL of phage dilutions 10-2 to 10-7 of production batches with a titer of 1E+11 pfu/ml in Sodium-Magnesium Buffer (comprising 5.8 g/L NaCl, 8 mM MgSO4, 50 mM Tris-Cl, pH 7.4) are spotted onto plates and incubated overnight at 30° C. Within the context of the present invention, a bacteriophage is said to infect a strain if a single plaque can be observed in any one of the spots.

For bacteriophages to be safely used in biocontrol of food-borne or feed-borne pathogens such as Salmonella, they should preferably be strictly virulent (avoiding lysogeny) and preferably comprise no known virulence factors, toxins and/or antibiotic resistance encoded in the phage genome, and the ability for generalized transduction (the transfer of host DNA by phage particles) should preferably be excluded (Hagens and Loessner, 2010). Accordingly, a bacteriophage according to the present invention is preferably strictly virulent (i.e., not temperate, thereby avoiding lysogeny) and comprises no virulence factors, toxins, and/or antibiotic resistance encoded in its genome. The absence of virulence factors or toxins can be assessed by methods well known by the person skilled in the art. In one embodiment, the absence of virulence factors or toxins is assessed by whole genome sequencing and screening for virulence factors or toxins. Virulence factors or toxins include, but are not limited to any type of toxin, antibiotic resistance gene, haemolysin, strong antigenic protein and/or inflammation factor.

Preferably, a bacteriophage according to the present invention does not demonstrate transduction activity, i.e. does not show any transfer of a host nucleic acid such as DNA to other host cells. Transduction activity can be assessed by assays well known by the person skilled in the art. A non-limiting example of such assay is here depicted in brief; two mutant Salmonella strains are provided, the first strain resistant to a first antibiotic and the second strain resistant to a second antibiotic. The first strain is infected with a lysate prepared from the second strain that has been infected with a bacteriophage according to the present invention. Transduction activity is analysed by testing said infected first strain on its ability to grow colonies on plates containing both the first and second antibiotic. When said infected first strain is able to grow on the plates containing both antibiotics, the resistance gene for the second antibiotic has been transduced from the second Salmonella strain to the first strain by phage transduction. Within the context of the present invention, a bacteriophage is said to show no transduction activity if substantially no colony growth, more preferably no colony growth, occurs in this assay.

Transduction frequency is known to be increased by mutations in rIIA, rIIB, stp and ac (Young et al., 1982). It is preferred that the bacteriophage according to the present invention features functional ndd, denB, rIIA and rIIB genes. Within the context of the present invention, functionality can be assured through a transduction assay, such as described here above.

Preferably, the source of a bacteriophage according to the present invention comprises or provides a bacteriophage, preferably an isolated bacteriophage, belonging to the morphotype group of the Myoviridae and is of the genus Felix O1-like phages, preferably bacteriophage Felix O1, FO1a or FO1-E2, more preferably bacteriophage FO1a. Bacteriophages Felix O1, and Felix O1-like phages FO1a and FO1-E2 are very closely related and for the purpose of the invention can be used interchangeably. Bacteriophage FO1-E2 has extensively been described in Guenther et al; bacteriophage Felix O1 has extensively been described in Felix and Callow, Whichard et al (2010), Hooton et al, Guenther et al and Marti et al. Preferably, a bacteriophage according to the present invention may be a mutant, chimeric and/or recombinant bacteriophage derived from Felix O1. The person skilled in the art may construct a bacteriophage starting from Felix O1, preferably from bacteriophage FO1a or FO1-E2, more preferably from bacteriophage FO1a, by placing mutations in the genome and/or deleting and/or inserting coding sequences or parts thereof into the genome using methods known in the art. Said isolated and derived Felix O1-like phages are herein referred to as a Felix O1-like phage according to the present invention.

Preferably, the source of a bacteriophage according to the present invention comprises or provides a bacteriophage, preferably an isolated bacteriophage, belonging to the morphotype group of the Myoviridae, comprising at least one feature selected from the group consisting of:
the genome of the bacteriophage is at least 100 kbp,
the genome of the bacteriophage comprises at least one polynucleotide encoding a polypeptide with an amino acid sequence having at least 50% sequence identity with an amino acid sequence selected from the group consisting of SEQ ID NO: 3, 5, 7, 9 and 11,
the bacteriophage receptor is Salmonella outer membrane protein C,
the bacteriophage can infect and lyse at least one Salmonella species.

Preferably, a bacteriophage according to the present invention has a genome that has at least 50, 55, 60, 65, 70, 75, 80, 81, 82, 83, 84, 85, 86, 87, 88, 89, 90, 91, 92, 93, 94, 95, 96, 97, 98, 99 or 100% sequence identity with the genome of Phage S16, deposited at the CBS Fungal Biodiversity Centre under number CBS130493 and represented herein by SEQ ID NO: 1. More preferably, said bacteriophage according to the present invention is Phage S16 deposited under number CBS130493. Bacteriophage S16 has extensively been described in Marti et al and in WO2013/169102. A bacteriophage according to the present invention may be a mutant, chimeric and/or recombinant bacteriophage. The person skilled in the art may construct a bacteriophage starting from S16 by placing mutations in the genome and/or deleting and/or inserting coding sequences or parts thereof into the genome using methods known in the art. Said isolated and derived S16 phages are herein referred to as an S16-like phage according to the present invention.

Preferably, the genome of an S16-like bacteriophage according to the present invention is resistant to at least 10, 15, 20, 25, 26, 27, 28, 29 30, 31 or 32 of the following restriction enzymes: Eco521 (EagI), DpnI, HhaI, Eco1051 (SanBI), HincII (HindII), KpnI, MluI, MpH1 1031 (NsiI), MspI (HpaII), NheI, SacI, SalI, OliI (AleI) Van91I (PflMI), PaeI (SphI), Eco881 (AvaI), MssI (PmeI), PvuII, PagI (BspHI), BseJI (BsaBI), Bsp68I (NruI), TaqI, EcoRI, EcoRV (Eco321), HindIII, Paul (BssHII), FspBI (BfaI) NdeI, MboI (all previous manufactured by Fermentas GmbH), SspI (manufactured by GE Healthcare), PacI, SwaI (SmiI), XcmI, Call (last four manufactured by New England Biolabs). Restriction resistance can be tested using any suitable assay known by the person skilled in the art. In brief, purified phage DNA is incubated with a restriction enzyme at a concentration, temperature and for a time according to the manufacturer's instructions after which RFLP patterns can be analysed electrophoretically.

Preferably, the source of a bacteriophage according to the present invention comprises or provides at least two bacteriophages according to the present invention as described herein above, more preferably the source of a bacteriophage according to the present invention comprises or provides:

(i) a bacteriophage, preferably an isolated bacteriophage, belonging to the morphotype group of the Myoviridae and is of the genus Felix O1-like phages, preferably bacteriophage Felix O1, FO1a or FO1-E2, more preferably bacteriophage FO1a, and
(ii) a bacteriophage, preferably an isolated bacteriophage, belonging to the morphotype group of the Myoviridae, comprising at least one feature selected from the group consisting of:
the genome of the bacteriophage is at least 100 kbp,
the genome of the bacteriophage comprises at least one polynucleotide encoding a polypeptide with an amino acid sequence having at least 50% sequence identity with an amino acid sequence selected from the group consisting of SEQ ID NO: 3, 5, 7, 9 and 11,
the bacteriophage receptor is Salmonella outer membrane protein C,
the bacteriophage can infect and lyse at least one Salmonella species.

Preferably, the preferably isolated bacteriophage (ii) is Phage S16 deposited under number CBS130493. Preferably, the preferably isolated bacteriophage (i) is bacteriophage Felix O1, more preferably FO1a. More preferably, the preferably isolated bacteriophage (i) is bacteriophage Felix O1, more preferably FO1a and the preferably isolated bacteriophage (ii) is Phage S16 deposited under number CBS130493.

Preferably, the preferably isolated bacteriophage (ii) is Phage S16 deposited under number CBS130493. Preferably, the preferably isolated bacteriophage (i) is bacteriophage FO1a. More preferably, the preferably isolated bacteriophage (i) is bacteriophage FO1a and the preferably isolated bacteriophage (ii) is Phage S16 deposited under number CBS130493.

In conjunction with the broad host range Salmonella phage Felix O1-like phages, an almost complete host-range can be achieved making a combination of a Felix O1-like phage with an S16-like phage uniquely useful for the various applications according to the present invention, which are non-limitedly listed herein. Accordingly, the source of a bacteriophage according to the present invention preferably comprises bacteriophage Felix O1 (FO1), more preferably comprises bacteriophage Felix O1 and bacteriophage S16 even more preferably comprises bacteriophage FO1a and bacteriophage S16.

Preferably, a bacteriophage of the present invention is none of the T4-like phages selected from the group consisting of JS98, JS10, CC31 and F387/08. Accordingly, the source of a bacteriophage according to the present invention preferably does not comprise a T4-like bacteriophage selected from the group consisting of JS98, JS10, CC31 and F387/08.

A bacteriophage according to the present invention can be propagated using any method known to the person skilled in the art. A bacteriophage according to the present invention is preferably propagated using a method as described in Guenther et al, Marti et al and Whichard et al (2003).

In a method according to the present invention, the source of a bacteriophage according to the present invention may be administered to the animal in any way known to the person skilled in the art. Preferably, in a method according to the present invention, the source of a bacteriophage according to the present invention is admixed with a feed. Admixing is herein defined as that the source of the bacteriophage is added, mixed or blended with a feed, using any method known by the person skilled in the art, before the feed is administered to an animal. The source of the bacteriophage may be admixed at any time point before it is administered to an animal, it may be admixed immediately before administration or it may be admixed and subsequently stored before administration to an animal. As an example, the source of the bacteriophage may be admixed to drinking water, milk or milk replacement or may be admixed to the water before the water is used for preparing a milk replacer.

Preferably, in a method according to the present invention, per administration about 1E+3 to about 1E+13 plaque forming units (pfu) of a bacteriophage according to the present invention are administered. More preferably, per administration 1E+3 to 1E+13 plaque forming units (pfu) of a bacteriophage according to the present invention are administered. Even more preferably, per administration about 1E+4 to about 1E+12, about 1E+5 to about 1E+12, about 1E+6 to about 1E+12, about 1E+7 to about 1E+12, about 1E+8 to about 1E+12, about 1E+9 to about 1E+12, about 1E+9 to about 1E+11, or about 2E+10 plaque forming units (pfu) of a bacteriophage according to the present invention are administered. Even more preferably, per administration 1E+4 to 1E+12, 1E+5 to 1E+12, 1E+6 to 1E+12, 1E+7 to 1E+12, 1E+8 to 1E+12, 1E+9 to 1E+12, 1E+9 to 1E+11, or 2E+10 plaque forming units (pfu) of a bacteriophage according to the present invention are administered.

Preferably, per administration, about equal amounts of bacteriophage S16 and a Felix O1-like bacteriophage, preferably Felix O1 are administered. More preferably, per administration, about 1E+10 pfu of bacteriophage S16 and about 1E+10 pfu of a Felix O1-like bacteriophage, preferably Felix O1 are administered. Even more preferably, per administration, 1E+10 pfu of bacteriophage S16 and 1E+10 pfu of a Felix O1-like bacteriophage, preferably Felix O1 are administered.

More preferably, per administration, about equal amounts of bacteriophage S16 and bacteriophage FO1a are administered. More preferably, per administration, about 1E+10 pfu of bacteriophage S16 and about 1E+10 pfu of bacteriophage FO1a are administered. Even more preferably, per administration, 1E+10 pfu of bacteriophage S16 and 1E+10 pfu of bacteriophage FO1a are administered.

A preferred method of feeding according to the present invention comprises a feed regime of three days wherein an animal receives two feeds a day, wherein each of these two feeds comprises a source of a bacteriophage, wherein the source of the bacteriophage comprises (for each feed) 2E+10 pfu of a bacteriophage according to the present invention, wherein preferably the source of the bacteriophage comprises (for each feed) 1E+10 pfu of bacteriophage S16 and 1E+10 pfu of a Felix O1-like bacteriophage, preferably Felix O1, more preferably bacteriophage FO1a. A further preferred method of feeding according to the present invention comprises a feed regime of five days wherein an animal receives two feeds a day, wherein each of these two feeds comprises a source of a bacteriophage, wherein the source of the bacteriophage comprises (for each feed) 2E+10 pfu of a bacteriophage according to the present invention, wherein preferably the source of the bacteriophage comprises (for each feed) 1E+10 pfu of bacteriophage S16 and 1E+10 pfu of a Felix O1-like bacteriophage, preferably Felix O1, more preferably bacteriophage FO1a. A further preferred method of feeding according to the present invention comprises a feed regime of ten days wherein an animal receives two feeds a day, wherein each of these two feeds comprises a source of a bacteriophage, wherein the source of the bacteriophage comprises (for each feed) 2E+10 pfu of a bacteriophage according to the present invention, wherein preferably the source of the bacteriophage comprises (for each feed) 1E+10 pfu of bacteriophage S16 and 1E+10 pfu of a Felix O1-like bacteriophage, preferably Felix O1, more preferably bacteriophage FO1a.

Preferably, in a method according to the present invention, the growth, depicted as weight gain within a specified time-frame, of an animal that is administered a source of a bacteriophage according to the present invention, is at least 1, 2, 3, 4, 5, 6, 7, 8, 9, 10, 11, 12, 13, 14, 15, 16, 17, 18, 19, 21, 22, 23, 24, or at least 25% higher than the growth of a control animal that is not administered the source of the bacteriophage.

Preferably, when comparing any method or regime according to the present invention, control animals are used of substantially the same age and individual weight as compared to the subject animals subjected to a method or regime according to the present invention. Preferably, control groups and subject groups of animals are used of at least two, three, four, five, six, seven, eight, nine, ten, twelve, thirteen, fourteen, fifteen, sixteen, seventeen, eighteen, nineteen, twenty, or more animals. Preferably the number of animals of a control group and of a subject group is substantially identical or is identical and/or the total weight of a control group and of a subject group is substantially identical or is identical. More preferably, the number of animals and the total weight of a control group and of a subject group are substantially identical or are identical.

To assay growth efficiency, depicted as weight gain, a preferred assay or study is to subject a group of animals to a feed regime according to the present invention, wherein at least one subject group receives a source of a bacteriophage according to the invention and at least one control group is subjected to the same feed regime but lacking the source of the bacteriophage. At the start of the study, all animals are weighed. A the end of the study comprising a specified period or time-frame, such as depicted elsewhere herein, wherein the method of feeding according to the present invention is applied, all animals are weighed again and the gain in weight per animal and per group is calculated. The absolute weight gain can be depicted, the relative weight gain can be depicted and the ratio between the weight gain of the subject group and the weight gain of the subject group can be depicted. The latter calculation will depict the efficiency of the method according to the present invention. The study period may be longer than the period wherein a method according to the invention is applied. Time points for measurement may be anywhere within the study-period; although at least two time points of measuring the weight of the animals must be used, more measurements may be included in the study.

Since the source of a bacteriophage according to the present invention can conveniently be admixed to a feed, the present invention provides in a second aspect for a feed composition comprising a source of a bacteriophage according to the present invention. Preferably, such feed comprises per administration dose about 1E+3 to about 1E+13 plaque forming units (pfu) of a bacteriophage according to the present invention. Such feed or feed composition is preferably used in a method of feeding an animal to improve growth efficiency. More preferably, such feed comprises per administration dose 1E+3 to 1E+13 plaque forming units (pfu) of a bacteriophage according to the present invention. Even more preferably, such feed comprises per administration dose about 1E+4 to about 1E+12, about 1E+5 to about 1E+12, about 1E+6 to about 1E+12, about 1E+7 to about 1E+12, about 1E+8 to about 1E+12, about 1E+9 to about 1E+12, about 1E+9 to about 1E+11, or about 2E+10 plaque forming units (pfu) of a bacteriophage according to the present invention. Even more preferably, such feed comprises per administration dose 1E+4 to 1E+12, 1E+5 to 1E+12, 1E+6 to 1E+12, 1E+7 to 1E+12, 1E+8 to 1E+12, 1E+9 to 1E+12, 1E+9 to 1E+11, or 2E+10 plaque forming units (pfu) of a bacteriophage according to the present invention.

Preferably, such feed comprises per administration dose about equal amounts of bacteriophage S16 and a Felix O1-like bacteriophage, preferably Felix O1. More preferably, such feed comprises per administration dose about 1E+10 pfu of bacteriophage S16 and about 1E+10 pfu of a Felix O1-like bacteriophage, preferably Felix O1. Even more preferably, such feed comprises per administration dose 1E+10 pfu of bacteriophage S16 and 1E+10 pfu of a Felix O1-like bacteriophage, preferably Felix O1. More preferably, such feed comprises per administration dose about equal amounts of bacteriophage S16 and bacteriophage FO1a. More preferably, such feed comprises per administration dose about 1E+10 pfu of bacteriophage S16 and about 1E+10 pfu of bacteriophage FO1a. Even more preferably, such feed comprises per administration dose 1E+10 pfu of bacteriophage S16 and 1E+10 pfu of bacteriophage FO1a.

In a third aspect, the present invention provides a method of treatment of an animal to improve growth efficiency of the animal, comprising:
providing the animal with a regular feed regime conducive to the growth of the animal, and
administering to the animal a source of a bacteriophage,
   wherein the source of a bacteriophage is administered to the animal in an effective dosage to improve growth efficiency.

In the method of treatment, the features are preferably those as described in the first and second aspect of the present invention.

In a fourth aspect, the present invention provides a source of a bacteriophage for use in the treatment of an animal to improve growth efficiency of the animal, comprising:
providing the animal with a regular feed regime conducive to the growth of the animal, and
administering to the animal the source of a bacteriophage,
   wherein the source of a bacteriophage is administered to the animal in an effective dosage to improve growth efficiency.

In the source of a bacteriophage for use in the treatment of an animal to improve growth efficiency of the animal, the features are preferably those as described in the first and second aspect of the present invention.

In a fifth aspect, there is provided the use of a source of a bacteriophage according to the present invention to improve growth efficiency in an animal. In the use of a source of a bacteriophage to improve growth efficiency in an animal, the features are preferably those as described in the first and second aspect of the present invention.

“Sequence identity” is herein defined as a relationship between two or more amino acid (peptide, polypeptide, or protein) sequences or two or more nucleic acid (nucleotide, polynucleotide) sequences, as determined by comparing the sequences. In the art, “identity” also means the degree of sequence relatedness between amino acid or nucleotide sequences, as the case may be, as determined by the match between strings of such sequences. Within the present invention, sequence identity with a particular sequence preferably means sequence identity over the entire length of said particular polypeptide or polynucleotide sequence. The sequence information as provided herein should not be so narrowly construed as to require inclusion of erroneously identified bases. The skilled person is capable of identifying such erroneously identified bases and knows how to correct for such errors.

“Similarity” between two amino acid sequences is determined by comparing the amino acid sequence and its conserved amino acid substitutes of one peptide or polypeptide to the sequence of a second peptide or polypeptide. In a preferred embodiment, identity or similarity is calculated over the whole SEQ ID NO as identified herein. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, BLASTP, BLASTN, and FASTA (Altschul, S. F. et al., J. Mol. Biol. 215:403-410 (1990). The BLAST X program is publicly available from NCBI and other sources (BLAST Manual, Altschul, S., et al., NCBI NLM NIH Bethesda, Md. 20894; Altschul, S., et al., J. Mol. Biol. 215:403-410 (1990). The well-known Smith Waterman algorithm may also be used to determine identity.

Preferred parameters for polypeptide sequence comparison include the following: Algorithm: Needleman and Wunsch, J. Mol. Biol. 48:443-453 (1970); Comparison matrix: BLOSSUM62 from Hentikoff and Hentikoff, Proc. Natl. Acad. Sci. USA. 89:10915-10919 (1992); Gap Penalty: 12; and Gap Length Penalty: 4. A program useful with these parameters is publicly available as the “Ogap” program from Genetics Computer Group, located in Madison, Wis. The aforementioned parameters are the default parameters for amino acid comparisons (along with no penalty for end gaps).

Preferred parameters for nucleic acid comparison include the following: Algorithm: Needleman and Wunsch, J. Mol. Biol. 48:443-453 (1970); Comparison matrix: matches=+10, mismatch=0; Gap Penalty: 50; Gap Length Penalty: 3. Available as the Gap program from Genetics Computer Group, located in Madison, Wis. Given above are the default parameters for nucleic acid comparisons.

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulphur-containing side chains is cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. Preferably, the amino acid change is conservative. Preferred conservative substitutions for each of the naturally occurring amino acids are as follows: Ala to ser; Arg to lys; Asn to gln or his; Asp to glu; Cys to ser or ala; Gln to asn; Glu to asp; Gly to pro; His to asn or gln; Ile to leu or val; Leu to ile or val; Lys to arg; gln or glu; Met to leu or ile; Phe to met, leu or tyr; Ser to thr; Thr to ser; Trp to tyr; Tyr to trp or phe; and, Val to ile or leu.

A polynucleotide is represented by a nucleotide sequence. A polypeptide is represented by an amino acid sequence. A nucleic acid construct is defined as a polynucleotide which is isolated from a naturally occurring gene or which has been modified to contain segments of polynucleotides which are combined or juxtaposed in a manner which would not otherwise exist in nature. Optionally, a polynucleotide present in a nucleic acid construct is operably linked to one or more control sequences, which direct the production or expression of said peptide or polypeptide in a cell or in a subject.

As used herein the term “heterologous sequence” or “heterologous nucleic acid” is one that is not naturally found operably linked as neighbouring sequence of said first nucleotide sequence. As used herein, the term “heterologous” may mean “recombinant”. “Recombinant” refers to a genetic entity distinct from that generally found in nature. As applied to a nucleotide sequence or nucleic acid molecule, this means that said nucleotide sequence or nucleic acid molecule is the product of various combinations of cloning, restriction and/or ligation steps, and other procedures that result in the production of a construct that is distinct from a sequence or molecule found in nature.

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”. The word “about” or “approximately” when used in association with a numerical value (e.g. about 10) preferably means that the value may be the given value (of 10) more or less 0.1% of the value.

The sequence information as provided herein should not be so narrowly construed as to require inclusion of erroneously identified bases. The skilled person is capable of identifying such erroneously identified bases and knows how to correct for such errors. In case of sequence errors, the sequence of the polypeptides obtainable by expression of the genes as represented by SEQ ID NO's 2, 4, 6, 8 and 10 should prevail.

All patent and literature references cited in the present specification are hereby incorporated by reference in their entirety.

TABLE 1
Sequences

| SEQ | | |
|---|---|---|
| ID NO | Name (GenBank accession number) | Organism |
| 1 | genome sequence (HQ331142) | Phage S16 |
| 2 | sequence encoding gp34 | Phage S16 |
| 3 | gp34; long tail fiber proximal subunit | Phage S16 |
| | (AEO97169; gp166) | |
| 4 | sequence encoding gp35 | Phage S16 |
| 5 | gp35; hinge connector (AEO97170; gp167) | Phage S16 |
| 6 | sequence encoding gp36 | Phage S16 |
| 7 | gp36; hinge connector (AEO97171; gp168) | Phage S16 |
| 8 | sequence encoding gp37 | Phage S16 |
| 9 | gp37; long tail fiber distal subunit | Phage S16 |
| | (AEO97172; gp169) | |
| 10 | sequence encoding gp38 | Phage S16 |
| 11 | gp38; chaperone for long tail fiber distal subunit | Phage S16 |
| | (AEO97173; gp170) | |

### DESCRIPTION OF THE FIGURES

FIG. 1 Mean weight in kg of the animals at the start of the study, at twelve weeks and at twenty-six weeks. The groups are control (group 4), 3 days treatment (group 1), 5 days treatment (group 2) and 10 days treatment (group 3). The mean weight in kg is depicted on the y-axis.

FIG. 2 Mean weight increase in kg at twelve weeks and at twenty-six weeks. The groups are control (group 4), 3 days treatment (group 1), 5 days treatment (group 2), 10 days treatment (group 3) and average treated which depicts the mean weight increase of all treated animals.). The mean weight increase in kg is depicted on the y-axis.

### EXAMPLES

The present invention is further described by the following examples which should not be construed as limiting the scope of the invention.

Unless stated otherwise, the practice of the invention will employ standard conventional methods of molecular biology, virology, microbiology or biochemistry. Such techniques are described in Sambrook et al. (1989) Molecular Cloning, A Laboratory Manual (2^{nd} edition), Cold Spring Harbor Laboratory, Cold Spring Harbor Laboratory Press; in Sambrook and Russell (2001) Molecular Cloning: A Laboratory Manual, Third Edition, Cold Spring Harbor Laboratory Press, NY; in Volumes 1 and 2 of Ausubel et al. (1994) Current Protocols in Molecular Biology, Current Protocols, USA; and in Volumes I and II of Brown (1998) Molecular Biology LabFax, Second Edition, Academic Press (UK); Oligonucleotide Synthesis (N. Gait editor); Nucleic Acid Hybridization (Hames and Higgins, eds.).

### Example 1. Efficacy of a Method According to the Invention

To determine the efficacy of a method according to the invention, a total of 66 animals was used in an in vivo study. The animals were of the breed Holstein and were two weeks old at the start of the study. In the control group, three animals died during the study period. In group 2, one animal died during the study period. The animals that died during the study period were excluded from the calculations. The phage composition used was mixed through the liquid milk replacer.

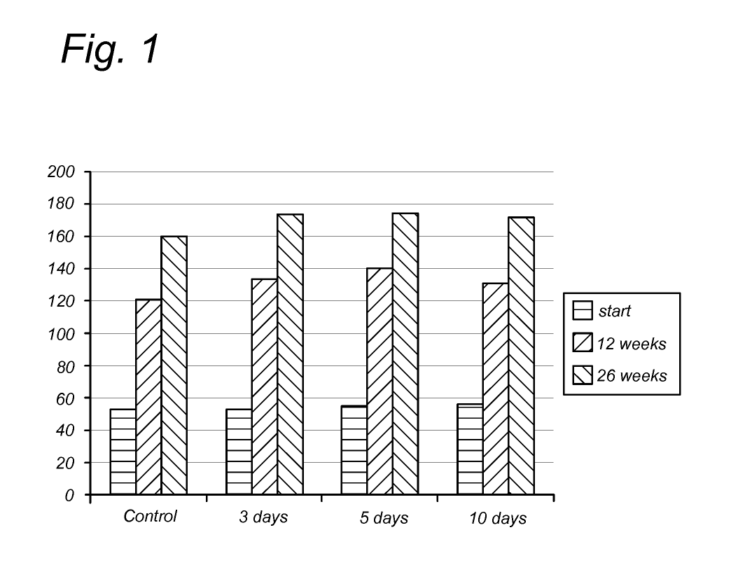

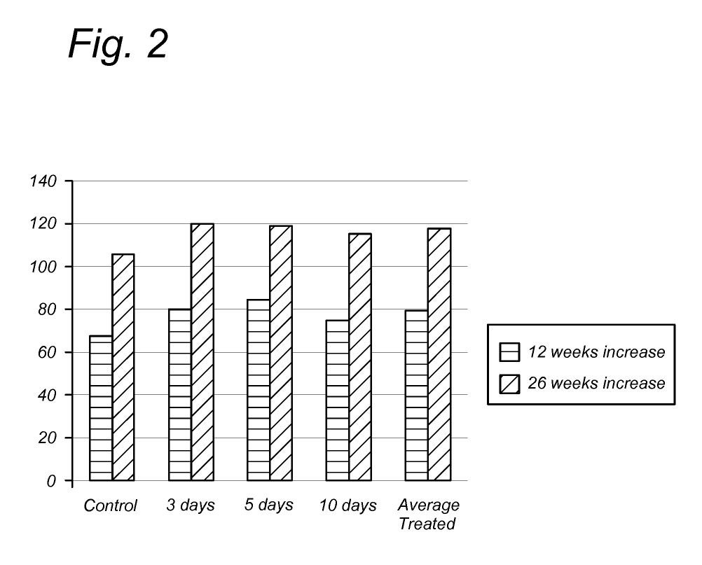

See Table 2 and FIG. 1 for the mean weights per group of animals throughout the study period. See Table 2 and FIG. 2 for the mean increase in weighs per group of animals.

Group 1 consisted of 15 animals and was treated for three days with 1E+10 pfu of bacteriophage S16 and 1E+10 pfu of bacteriophage FO1a per feed, two feeds a day; the treatment was started within 12-36 hours from the start of the study at day 0. The mean weight at the start of the study was 53.6 kg. At twelve weeks, the mean weight was 133.8 kg; at twenty-six weeks, the mean weight was 173.4 kg. The mean increase in weight was 80.2 kg at twelve weeks and 119.8 kg at twenty-six weeks.

Group 2 consisted of 15 animals and was treated for five days with 1E+10 pfu of bacteriophage S16 and 1E+10 pfu of bacteriophage FO1a per feed, two feeds a day; the treatment was started within 12-36 hours from the start of the study at day 0. The mean weight at the start of the study was 55.5 kg. At twelve weeks, the mean weight was 140.3 kg; at twenty-six weeks, the mean weight was 174.3 kg. The mean increase in weight was 84.8 kg at twelve weeks and 118.8 kg at twenty-six weeks.

Group 3 consisted of 18 animals and was treated for ten days with 1E+10 pfu of bacteriophage S16 and 1E+10 pfu of bacteriophage FO1a per feed, two feeds a day; the treatment was started within 12-36 hours from the start of the study at day 0. The mean weight at the start of the study was 56.5 kg. At twelve weeks, the mean weight was 131.3 kg; at twenty-six weeks, the mean weight was 171.9 kg. The mean increase in weight was 74.8 kg at twelve weeks and 115.4 kg at twenty-six weeks.

Group 4 was the control group and consisted of 18 animals; the mean weight at the start of the study was 53.7 kg. At twelve weeks, the mean weight was 121.4 kg; at twenty-six weeks, the mean weight was 159.5 kg. The mean increase in weight was 67.7 kg at twelve weeks and 105.8 kg at twenty-six weeks.

The combined measurements of the three treated groups were as follows. The mean weight at the start of the study was 55.3 kg. At twelve weeks, the mean weight was 134.8 kg; at twenty-six weeks, the mean weight was 173.1 kg. The mean increase in weight was 79.5 kg at twelve weeks and 117.8 kg at twenty-six weeks.

It can accordingly be concluded that a method according to the invention results in a significant increase in growth efficiency; namely where the control animals demonstrated an overall increase in weight of 67.7 kg at week twelve and 105.8 kg at week twenty-six, the treated animals demonstrated an up to 17.1 kg higher mean increase in weight. Overall, the mean increase of the treated animals was 11.8 kg and 12.0 kg higher than the control animals at weeks twelve and twenty-six, respectively.

TABLE 2
Mean weight increase per group of animals and difference in mean
weight increase of treated animals versus control animals.

| | | Group 1 | Group 2 | Group 3 | Groups |
|---|---|---|---|---|---|
| | Group 4 | 3 days | 5 days | 10 days | 1-3 |
| | controls | treated | treated | treated | treated |
| Number of | 18 | 15 | 15 | 18 | 48 |
| animals/Group | | | | | |
| Mean weight | 53.7 | 53.6 | 55.5 | 56.5 | 55.1 |
| at start of study | | | | | |
| Mean weight | 121.4 | 133.8 | 140.3 | 131.3 | 134.8 |
| at week 12 | | | | | |
| Increase in | 67.7 | 80.2 | 84.8 | 74.8 | 79.5 |
| mean weight | | | | | |
| at week 12 | | | | | |
| Increase of | — | 12.5 | 17.1 | 7.1 | 11.8 |
| treated versus | | | | | |
| control wk 12 | | | | | |
| Mean weight | 159.5 | 173.4 | 174.3 | 171.9 | 173.1 |
| at week 26 | | | | | |
| Increase in | 105.8 | 119.8 | 118.8 | 115.4 | 117.8 |
| mean weight | | | | | |
| at week 26 | | | | | |
| Increase of | — | 14.0 | 13.0 | 9.6 | 12.0 |
| treated versus | | | | | |
| control wk 26 | | | | | |

### REFERENCE LIST

1. Felix and Callow, (1943). Typing of Paratyphoid B Bacilli by Vi Bacteriophage. British Medical Journal 2: 127-130.
2. Guenther, S. et al., (2012) Biocontrol of Salmonella typhimurium in RTE foods with the virulent bacteriophage FO1-E2. Int J Food Microbiol 154: 66-72
3. Hagens, S. & M. J. Loessner, (2010) Bacteriophage for biocontrol of foodborne pathogens: calculations and considerations. Curr Pharm Biotechnol 11: 58-68.
4. Hooton et al., (2011). Applications of a bacteriophage cocktail to reduce Salmonella Typhimurium U288 contamination on pg skin. International Journal of Food Microbiology 151: 157-163.
5. Lindberg, A. A., (1967) Studies of a receptor for felix O-1 phage in Salmonella minnesota. J Gen Microbiol 48: 225-233.
6. Lindberg, A. A. & T. Holme, (1969) Influence of O side chains on the attachment of the Felix O-1 bacteriophage to Salmonella bacteria. J Bacteriol 99: 513-519.
7. Marti, R. et al., (2013) Long tail fibres of the novel broad-host range T-even bacteriophage S16 specifically recognize Salmonella OmpC. Mol Microbiol 87: 818-34.
8. Whichard et al., (2003) Suppression of Salmonella growth by wild-type and large-plaque variants of bacteriophage Felix O1 in liquid culture and on chicken frankfurters. Journal of Food Protection 66: 220-225.
8. Whichard et al., (2010) Complete genomic sequence of bacteriophage Felix O1. Viruses 2: 710-730.
9. Young, K. K., G. J. Edlin & G. G. Wilson, (1982) Genetic analysis of bacteriophage T4 transducing bacteriophages. J Virol 41: 345-347.

What is claimed is:

1. A method of feeding an animal to improve growth efficiency, comprising:
providing the animal with a regular feed regime conducive to the growth of the animal, and
administering to the animal a source of a bacteriophage,
wherein the source of a bacteriophage is administered to the animal in an effective dosage to improve growth efficiency.

2. A method according to claim 1, wherein the source of a bacteriophage is administered to the animal at least once, twice, three times, 4, 5, 6, 7, 8, 9, 10, 11, 12, 13, 14, 15, 20, 25, 30, 40, 45, 50, 55, 60, 65, 70, 75, 80, 85, 90, 95, 100, 110, 120, 130, 140, 150, 200, 250, 300, 350, 400, 450, 500, 550 or at least 600 times within a period of at least 1, 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 250, 300, or at least 350 days, wherein the source of a bacteriophage is preferably administered at least once or at least twice a day.

3. A method according to claim 1, wherein the animal is from the even-toed ungulate family Bovidae, more preferably from the subfamily Bovinae, even more preferably from the genus Bos, even more preferably from the species Bos taurus (domestic cattle), even more preferably from the group of dairy cattle or beef cattle.

4. A method according to claim 1, wherein the animal is a new-born or a juvenile animal.

5. A method according to claim 1, wherein the source of a bacteriophage comprises or provides an isolated bacteriophage belonging to the morphotype group of the Myoviridae and is of the genus Felix O1-like phages, preferably bacteriophage FO1a.

6. A method according to claim 1, wherein the source of a bacteriophage comprises or provides an isolated bacteriophage belonging to the morphotype group of the Myoviridae, comprising at least one feature selected from the group consisting of:
the genome of the bacteriophage is at least 100 kbp,
the genome of the bacteriophage comprises at least one polynucleotide encoding a polypeptide with an amino acid sequence having at least 50% sequence identity with an amino acid sequence selected from the group consisting of SEQ ID NO: 3, 5, 7, 9 and 11,
the bacteriophage receptor is Salmonella outer membrane protein C,
the bacteriophage can infect and lyse at least one Salmonella species.

7. A method according to claim 6, wherein the isolated bacteriophage is Phage S16 deposited under number CBS130493.

8. A method according to claim 6, wherein the isolated bacteriophage is Phage S16 deposited under number CBS130493 and bacteriophage FO1a.

9. A method according to claim 1, wherein the source of a bacteriophage is admixed with a feed.

10. A method according to claim 1, wherein per administration 1E+3 to 1E+13 plaque forming units (pfu) of a bacteriophage is administered.

11. A method according to claim 1, wherein the growth, depicted as weight gain within a specified time-frame, of an animal that is administered the source of a bacteriophage, is at least 1, 2, 3, 4, 5, 6, 7, 8, 9, 10, 11, 12, 13, 14, 15, 16, 17, 18, 19, 21, 22, 23, 24, or at least 25% higher than the growth of a control animal that is not administered the source of the bacteriophage.

12. A feed composition comprising a source of a bacteriophage as defined in claim 1.

13. A method of treatment of an animal to improve growth efficiency of the animal, comprising:
providing the animal with a regular feed regime conducive to the growth of the animal, and
administering to the animal a source of a bacteriophage,
wherein the source of a bacteriophage is administered to the animal in an effective dosage to improve growth efficiency.

14-15. (canceled)


SEQUENCE LISTING (11 sequences)

SEQ ID NO: 1 - DNA, 160221 bp, Unknown
  Salmonella bacteriophage S16
aattacctcttagatggcgccctggagcgccgcaattttcatgatgtcagtgtagtattcttctcgagaaatgtagtattcttcaagcatataccaaatcactgggtattttttagcaaattcgtcagaagctttattaaaatcagactttgacgataagatgagtttattataaatcttataacataaagcaattttttcagcggcttctgactgatttactgttgtgttaatagagcggtaatttaatgcttccgctatacggttaagaactaaagacttttcccagttatcaccggagataaaattcttcagcaacggattaagcgcttttatattaatcatagcgttgaagcgtctgatattaaacaaatgtggatgaattaaatcatcaagtacgtcgtcaatcaaatctgcgtaactatcagctaaaacatcaaaaactgaagttagagaattattgtctttgacatatttcattgcgctaggacgaatagtataaaattcttttatatctgccgctttacatgcttgtctaatattttcaatgctgtaactcgtagagcattctgttcttaattcaacaatgtcttctcgattaattccaactgctaaaccttccaattcacgaacttcatttgcagttaaaaacaaattagtcgattcccaatgaagttctgaatccaatttccatagatgaacattcggagattttggacgtttaaccgcatctgaattatctggcttattgatttcagcatctttggcacgggcttgttccatatcagaagctttaaaaataactacttcatcgccttcaaacaacttcttggcaagttctacaacttctaaatggtcttcattattcgggtcaattaaaataacttgacagcctttatgaatattaaggtaactcatcccacgaatagtgcttgctctgcggtttggcttatcgtcaatcataaaaacaatctttttctgattaacatgaattaagccagatgccgaatatcgattacgcgtgctccatgaattactaattttgcggcgttcagccatatcgccgacaaagtaagtatacatattagatgaatacattttattttcgagagcacgttggtcgaaataatcaagccattcgcggcaagttttgccttgaatttttgattggctattaccaataatagcaagagacctgctggaaaatttctttaactcacggaccagttctcgcttattggttatactttcaagatgtttgatatcgtctcttagatttgtttcttcgattttattaattttattgcgaatagcttcgatagtttcttcattgtaagaaagctcttcacggctaggagtaatatcaagttcacctaattcaaaattaacataaaccgtgctatacttgttattcagccattcgcatttgataccaggaacttcttgaaccttaatcggataaacaattcgtccataaattgcatacaaattcgaatcagtatcaaaataagagcgttgtttattaaaccattgcttttctggagtgaactcttcgaaataatcaatattgatattagcaccttcaatttctggttcaacaccagcaaatgtgcgcaaagtacgacggacttcatgatgccacttatcaatatcgtcagtttttacagggacagtaatttcaataccagtgtattcatcttcttccattgctgtgtcaaacagcggaataatttctggaccagtatttttcattatagcggtgtaaccgcgcttacggcctttatgacaagaaacgactgtaaaagtttttgtatagctaaatggagattttgagccaagacccattgccccgatgaaatcatttgagtcggctttagttgaagcaaaataagtattatacaagcctggagcatcatcagttccacgaatttgttcatcgctcatacctgggccaaaatcgcgaattacgaagcgcgggtctatacgagtaggaactttaatagtgaacggacgagtttggccattcagtttatgagcatcaagacagttagtagataattcacgaacgatagcgtaaattttattggtatataacttgtctgaaagaaccatgaacgctttcatgttgttctcaatgccaaacttacttgccttctgattggctgaaccaaaaatctgctcttcttcttgtttaataatcatctttttctctcattcacttaaattaatcttggttacgcttaatcaattctgcaacttctagaagttcttctgccgttgcggtgttgttttgtattttaaaccgtatttccttaaagcgttctttaaaatcttcagcttcactcatgtcaaaaaatctttgaatgatgcggtactctttatatacagcgtcatcaaataataccatacaaacgttaaagcttttcatttcctacctctctgaggatggctgcaaactggctcatagtgtgtttttcacgtacagagtaaaccagcctcaaagcggtttgttgtaggttcaccggaatgatctggttgttttcaactaatacagcaatatgttctatcatattagagaatttatttaaccagatttcatctggtgtattctctgttttattcagaaacatatctttactatctctaattgttgctgctacgttgtataaattaacataacttccgttaagcattttcatagctgtaatcaccatactaaaaagggactcccgaaggagtccctgttattaccatgtggcttttgacaccatacctgtaactttcttcaaagagttgtccggctgacgaacacgaatagtagccatatcaccagggagaggaagagcatctccccacatataaccgaaacttccatctacaagcgttacccagatttgtgacataaactctcctatacgggcggaatacccgtattatttattcactcatccagaccttgcgaggagcagcatcattacccataaccatttcaaaaagttctttccaatttggaggtaatgaaactacatcatacaccggcttctggataactcgttcatactcatcttcttctaaagaaccaagtcccttgatataacgaagtttccatcctgtgagtttatctttttcagcttcatactcagcggcattgtaataccatttttgctcagagcccttggacataataatcaccggggttttaacaaaacgaatacgaccttcttcgaacaacttcggccaattagcaaaaaatgctaacagacttggataaattgaccctgtgccaaatatctctattgtgaggtatatcaatgattagcccgaaggctaaccatcaacatcagcatcggtcatgatggcgatgtttttataattcaaagaagaagttgatgtacggcgtacgattgtctgggctttatatgacttcatatcgatttcagtttctttaactttagcaaatttcatttaaaacctcataaaatttttcatcaggagtccaaccgtttttaaactgattgaacgcgattttgaacagtttcgttttgatagttatattattaaccgttttccatttagcaattccggttttcttataactaactgggtcataaacgttagcataccaatcataaagggacggaatcaacttaactgcgtctggattggttttagatttgttataccaaggtttagatttttcaagggcggctgctacttttgtagcgtttaacgtgttaccaactccgcataaaccaaggtcctttattaaagggtcgtccgttcttagtctataggttgtgttatctttatccttgtaaactccgtatcctttcacaccttcaaattttcttccgtaatttacgccaacccaatcttcttcagtgtattcgcctttcgtcagtaatcgacattctccagtttctttatttctaagtaaaattttgtcacgcatgaactcagcagaccaatctgaaatcatattaacataatttttcctggctatttcataaagtttagaacgaaatattgttccagaattcattatccggttcaaagcataagacatttctttgtttttatgaattttaaacaataaccagtgcgcaataaaatgttcgcgatatgtcaattttaccaaattatctgaagtatctaatccacccatgcttcttggaagtatatgatgaatttctcctttaaccggcggaaccctggttttaccccgggaaattagtttttcatatatcaaactataattcatactaagtcttttacttttttccagacgccatcaataaaaatttcgtcattgatattaactacaatatcttcaccggcaacatttacagtataccattgttcgccggcttcaacttccgtgaatgggtcttcaccaatgactaacccagtaattgcacaaatatcaaagatttctttgtttttcaaagcatcacctgcagacattccccaggtgttcatcactttaccacgtaatggataaccgccatgaagggcacggtcacgagtattaattagataaccaatagcagaatcaccttctgttaagaacaaagttgtttctactttatcattaccatattcaccagctttgatgtgtttatgaactttagctttggccgcctttttcgctgctttggtttctgctgccttctcagccgccagtttacgtgctaacgcagcttcaataatcggcattaaaatagcatcagatttcataatctgattagaaattttctttgcatcgagatttaagtgagatttaatttcgccccatggacttgtaagacgctctttggtttgcgagtcaaagcgcaagttactcatatctcgaataaacatcaacacagtaaaacactctttaatacgagctttgtttacttcaattttgtgtttacgtttaatagccggaattaattcattagcaatttcgtcaataacataatcaatatgcgaaccgccattctttgtatgaatgttgttaacataagtcatttgacgaaaaccatctggagaattacctatcgccaaagaataattttcttcatcaataatgatagcatttttatcatactgcttagaatattttttgaaattactatcaacttttttattgttaaatttaaattcaatttttggataaacgactgccaatgtctgcaaacggtccagcataatatcaaaataaacatcatcaaaattgttcatttcgaaatggctaaaatctggaataaattctaccttagttcctttataagaggattttttggatttccaagaaacattgtcagaattattgctacaagaaactgttaattcattttctccatcagatgttgtacctttaaacataacagagaaaatattagtcaatgaagaaccaacaccgttttgcccgccagttttacgctcttcatcagctccaaagtttccgcctgccttagttttcgtccacgctgctactgggccaggaatttcttcaccctcaggagtagtaaccatagcctgtggaattccacggccattatcttctacggtcactttgcttgtggaagaatcgatattgactgaaattttattagcaaatttaaaacttgtacggatagcttcatcaacagagttatcgataatttcatcaataagtttgagaacgccagctacgtattgtacccgcttaaattcgccaaaaacaaaacgctcatgaaattcgttagatacacttccgatgtacattccaggacgcttaagaacgtgttccttgtcagagagcatttgaattttattttcaatcatgttattatcccagtttgaatttgtctgataattatatcatcaatttaatttagcaattaattcatcacgatatttcatgagatgatgaagcgccggagccccatatataggagaacacaaacttgaattaggttttttacaaaatatggtgtgaatagcttgctcttcactttcgtatgaaaacgtcaaagtttggctataccgatcatcataataggcagcataattattttcttcaattacgataaattcttgaacaactttataatatttttgtaacaagatgtaccgtccgttatccaatttagtcgggaacaatgcgaatttatattttccaatcgctcgttcaactaatctaagcttgcgggcggcattcttttgaattgaagattttccaaatatcataatatttttctcactcagccgttaagaataatcgcgatttttgtatccgttgtaatgaacagcgggcgcagcggtttctcggaacatttttccttcacaattcagaaatgaacattcaattggttcatcacgttctgagaccttgcgcatcttttctaccttttcaccgcaggcttcgcacgtgtaatcataaattggcattatagtaatccttttaacatttcaatagccgctttgacttgatttttattagtaattgctaaagtgatatgaggaatttcattttcttctgaagcttcatattcgtcatcattagccgccgaattaagtgtttccaacttaaaatatttagcttcgtcaggcaagataaagaaatattcattactagttttaataccagaatattctggtattttgtcaattatatcttgcgcgccgcatagagcctggcttaatgttataattgcttcagcggagccacaccaattaacgcttaaaactttaaaaaatccgccatttttattaatgaaattaataattgatttgttgtgtctatgagctttgacaaatccgtcgtaatctttaatagaataccattgacctttaacgaaatgtgttaaattctgtgtagtcattagatagtttccttccaataagaagttccgttttcattgatgtaacgaactaccgcatagattgcacggctgttttcgtaaacgtaggcgtatgtatgatgttctagatgtccttgaacttctttttgcacaatagtcaactctgggttgaacaagatatcttcaaactgctcttgtgttaaaatgcggttcatttattttcctcgtttgttttgatagggttatagtatcataaccctcaaggaaagtaaacagtttaaactttcacgctcagaaaacggcttgaacggattactgctgggataactaaatccaaatctttagtagccattaaagcgtattcaccctgataaaccacgttcgcaacttcttcgccgtcaatatcataaaccaaggtaactaatccacacttacggtcacaaatgtgacagtctccctgaccgccggaatgctcgattgctgcatctgcgtttcgaattcgtacaatatcgccaatttcaataacacaacgctgagaagcttctttcgtataagtgtcgttttcagacacatagtagtcttttacataaattgtgttattagttttaacatcgcgcagacgatatccgtcacgctttgaatgacctagaacaataccaacacgaccagcatttttaccactaattacacgaagttcagtacctttagcaaacatattatcacctttcaattttcagatttgagaaattattttatattaaaatatttaaagcgttatccacgaccttttggaagtgaaagagagtcaatttcagcaccgaccggagcttcttctaagccaagcgcatatcgttgagcatacttcagcatcagaatatctttagcacaatcatggatactatcatgagcgatgaacccgtcaagtacaccattacgcaatggagttgttgttaaatttctagtcatcagcagagcttctattgctgtacggatatctcgctggttccagaatttacatggttccaaattaaatgtgtcaatatctttatcttcaattcctttccggagttcaccttcacgtaggatatcaacaagaataggaaagtcaaatgactgtccgcggcaccatccgaatgagttccatgcatctactccgttatctttcaggaatttaagaagtttataaagtccttcgacgtggtcaatgtcatcatcagacggtgcaaggttagctctagcttcagctgactgctttttccaccaagctaaagtacttgcaccgaataaccgaaatggacgctgagaccggaggtcaaactttagtttcattcccctagaaactagttcgtcgaacgtttcgataacttccggattagggtcaaaaactaccaccgcacagtcaataactgctgatcgagaaacgttaccgaatgtctcccaatcaataataaaatcttttacgcccggcatagatattccttaaattcattaatctgtttagatattttatcattaattgatataagcggtacttgtccttggaaccaagcgatactcatagcttcgtcgcgaatctcgtttagttgaagcaataacgtagattgatgaccgcgccaccaagcatcttcgggtttgttaatttcaatagcattaatcaacttatcaaaatcatcaatatcaagagtaaacatatttgttccttatacaaagaaaacatcatggcggccacgagttgcacccacataaagaagttctagcttaaacttattatcgctgcttacgtgaatacacggagtataaatgaaactactatctacagaaataccctgagctttatggaacgttgaacaaggtaaagctttaaccttatgaaattttcgtttagcatcccagaattctgaccaaggagctttaccgcctttattccagtttttataagtatctgccgtctttgccaagaaaaactgaaatttgttcatttcttgttcgtcagatattacattaattttctcacgagcatattcttcatcgtcataagtttctacatctaaaacccagtgacgaattagatgttcaccagatactccctttgctcccaagaatgaagatgtgtaatctgcacttagaatacgaacatattgaccattagtaaataatgtttcatggaatttttttccctcaaatacaagttccctcattaaaggttcttgcataacaataacttctcctactacgaacgcttcttccgtttgatataaacgtctacgaatgatagagtttaatttatcaaccgacttatttgtgaatgcgagcattcggttttcaaacaaatcttccggtgatttgacgatactaaaatattgcatcataaaatctttaagtgccgttgtacttgtaaatccatgcacaccatgaccatcaacagttttttcgtaaatccatgaaccgtttcttatatcagtagcaacatcaataatcggagcgttactgcgcattacttcagtaagatttaattgtttaaaatctttatgaataaagaaaggagatatatgagcttctgtttctccagggtcaaccggacgaatttgagcgacatctccaatagcgactatagtacaccatttaggaattgaagccattagtattttaaataatttgcggtcccacattgaagcttcgtcacatattagcactcggcatgatgctaaatctggcacttctttttgttcaaacaacatactttcttcatatgttgtggggttaatttttaaaattttatgaatagtattagcttccatgccggacaatttggttaatacttttttggctgcatgtgtaggagccgtcaaaataataccagtttcgccagtagaaactaaatgctccatgataaattttgttaacgtagttttaccagtacccgctggaccgttgatagtaacatggtttttcttttcttttatagctctaatagtttcatcaaatgctagtttctggccagaagtcaactgctcaaatgtaatcataccactgatacccttttaacacttaatgtgttcataatttgtctgttcattctactttcaatttcagaaagctttttaaacttcatttcaactttatcgaaccagaaacgacgtttcttaattgtagtttctactttaccgacattcaatatatgagtaagataagtttttagctgctcatagtgcttcacagcatcatcaaatttttcatagattgtagcctttccagattcgaagtacgggagttcaaacccgtctttatctttgtagactaacatgtatccatagtgcatcacattttgcctcggataagttgtaacaatgttgcagaaagtggaagaccttttttcagcttcgctttatccaaacgagataaattttggaaacgagtaagaccttgaataataatatcataatctttcacggtgatttctgctttgctattctttttggatgcagcgcctgaagttacagcttcagaagacgggtatttttcaaatattctctttaatgttttaattccgcctttgatgcgagtttctagagaatcgcgagtgtctttctgtattccggcttgcttaagatgactgcggcgggcctgcacgtttgcccaaatgcgtttagcttcttcacggcggacaccggaaattttagaccaatcaggctctttgttccaatcatgagtttctaatacaaagtcagaatgattgaggtcaacagcaggttctacaactttgtttttagagtcaacggcggaaacgatgcgcgcagctagttcttcttgctttttaatgaggtcatcaatttcgaagttggcgcgcagtttattgtacttcattttaacaggaataacgttgccttctacatatcctttgttgttatcaaaccgctcgagagtcatttggtcaggcccaagttccttcttgaacttttctcctgaatatgcacaagtatcttgctccatgatgttcatcaagtacttcaaagaaagattaaattctttacttctagaagaagcacttttgtatgtatgaaccaaacgttgagcgataacaatttcacgaggcatggccataacgatattccttatttgctttttatgcgaatgtggtataatggtttcaaggattattctaatacactctatccggaaagtaaacggttagaatgaataaaaatccaaatgatatacagtatatgacggagataaagggtcatactgtaactcttcaatctccactatctcaaattgtgttccaatagggaacatccattcatcttctccattgaccatttcaagtttatccagtctttcatcaaaaataaaatctgggtctatgctgtgcataaattctgtgtctggcgcagccagaagcatattcagtgcgtgctcttgatagttaaaagcaaatgggcaattacgtaatgacaaaatagtatgagtaccatacacactccctccagcgaattgtcttgctgtggaaaaatctgatgtaaacgacgtaactcggtcatcagcaataattcttcctacagccatatttttcaaccaagcttcggtacgttttgacactcctctgtagagttcaactggaacgtcagaactaatatgcttacgtataattttgtccagttctacatgcaaagttttgtcaattttatcattcatacactgccaaagtatgctctgttcaaaatcactgaacatcttaattttttcttcatacaatccatagagattgttatcaactatgttacaaacgatttcattaagctgagaatttgtgtataacataatagcctccgtagtttacagaagctatcataacacattttgactgggatgtaaacggttattctgtccacggagcccaaggaggagggtctaaccagatagaagagagttcttctacgaaagtgtcgttgagttttacaccatgccgtaaggcaagaacaaaaagctcttgtttgatttcagcacgtcggttgccaaatgcttctgaattttttgactggtcttcttcaaataaatcaaataaatccgacattacatacctctgatgtatttgacagcagttacggacatagttttttcttcaaatccattacgacgaattccagtatcattaaaatgatgccaaatgaatttttccaattcaacgatgtgttcaggagtatttacaataaattttttgaagatttttttgccatatccatctgtacgacacaaaggttcgctaaaattgaattccggaaaaatgtatttctgactaagcaaagtttctaccataccaaacggtgaattttcgcatttgtcgatattacaatcatgaatgatttctaacaatgcattttcgggaattcgcattttcataaactgcttttctagattattataatgctcgtttgaaaaaggtgcaggttcaccatactgagttagaacaacttcaaacttttctcgagtttcttctactcgtcgattagttcttttatagaatttcactacaaatgactttgttttcatatttttcatgatattctcttattttatgacttatcgccgtaaatgttagttaaaagctttttagcttctattgaaatacgtttagcttcttgttcattgcgccaatttctagcgtattcttccgcttcatgttgaagttcttgctcaactttagcaacccattcatcatctaaatgagtaggtgtttcaaaccaatttttgaattcttcgtaccatcgtttaatcataaccaggatgtcccaaagcgaataacagatttttcacctacaaagtgaaagtgagcctcatatacgacacgtaaagcagtaccttcctcacggtagcaatgaataaagatgtctactaaatcatacagatttgggctatttgcgataaacaaatgttcatgaattaagcgatttgcaataagactattgataccaccaacttcttcaactggcttcatccagttagatgttttcataatattatcctcatttgataaaagaattatatcatatcgtttaaaagcaaaaaagggactcccgaaggagtccctgcaatcaatcacctggtactactggcggtacgcctttaaacatatcagcgtttttggcaatccaagcgtctctttggtcctcttcaaaaacggtagaatacgccgctttttcggaagggaagagttgataatgagaaccagcaatttgatgcacgtcggagtaaagcttaaaagctacagaaacttccataccttttacttcaccgtcggcatttgtatggtcaaacgtttttacgttaacataactcatgtaattctcctttgttgattacatgagtatttattaatagttatttaaagcctctactatatctgctatgatatcttcagaatcgccaataagttctcgaagagatttgtacttatcttctggacttagaggatttaagccccaatattttattaaagcattttcaatatcagaatatgaaatcattagtatcctcggttttgccgctcaaagttttcagcatttttcagatagtaaagtttaaagatttcttcagccgacatgcctaatccttggaacatattcaacacaaagtgaagaatatcaatcatttcaaatttaatttctaattggtcttttgaagacatttcagaaataagacgattgcgtaattctgcatgttgtgctttccacggtttccagacagaagaagcagccttctcgccattagacataccgcccagagaagtcaacagctcacggaattcgtcatcgatataatctttctgattacgtagccaatcaacaacttcaccagctttagctaagtcatctggattgcggttatagtcaggtttatcatttgctagacgcacttgcaatgatttttgcatgtcaagcatcacttgaagcgggtcaataccagcattcacagcatcataatacgcattttctgctttttcattgccagagatgagttgagaacattcattaaaatgggccattatatttcctgtcaaagtattaaatagtttaggaggatttatgtctgaaaaaattaaaaccccagaaaataaaaatctgaagtctcaattcgttgagaacaaaggtaaattaatcttagttggagttattacagctgtaataacagcatggaactatattatcatccctttcgcattagcatatgggtttactttacctccaattccattggataaagttattcatttcattatgctcggaggattttaaactccaatctttgagtaattcttaaggcgaagttgagccatcaaacccgaagatatattttcggaaatatatttttcgatgtcttcaactttcgccccttcattttttatcatatcattaatgtctttttctttccattgagatttatcccaaaacacaactctttcgccagcgtctatcaaacgcttcattcgcttgattgtgtcttcttttcttggttcatggtccataatccatactctatcttctttaaatggaaccaaatccaaatcaatagcgccaccggtgatggcaatagcattgcccaagaacaatgagtctatcgggccttctaatacaaaaatattttgaccggtctttgctgtgtctaatccgtatatttttgttgcatgttcgtttcgtttaattgttatgtatttttgaggggcgtctttacgcaatgctcttccttgaaaactttcaataatacctttctcattaaagattggaattactaaacgaggctcattacgttctgttttataagttccgggattaacggaattcaccaaagctggccattccatcgtaaaatacaaacggttccaacgcgattttggaatacaacgatgtttaacgtattttataataggatgctcttctgacaaactatctaagcggctgcaatgaactaatttaattccttctggttctttaatttcaacttttttcacctctggttttgtatcaggcttaaaagtcattttgtcctttctgagttctaaaataaactcacggtataaatcaggttcatattcctttaaatacaccccaattggttttccataatcacagttataacacttcaaaaatacatcatcgcccgtgccataagcccaaaaacgagcttttaatgcgtctttctgtgagtcgccacagactgggcagcggcagtttaacttgaattgggatacgttgtttacttgacgaaagcgaggtaagtgagataatgcgcgaaatgcaaactcattgtgaacccaagacatatattctcctaagtggccttatttctaaggccattatattacaatttcttaagattaactttaatgcgcttacgttttgaggggatttgttctggacccttgttggttattgctccggttgtggtaccagaggcgatattctggacacttccaccggaatcaccggctaccatgtcctcgaacatagggagagcatcaaatatctctttctgttcggattcggttatattgtatctagaagcgattgaagaccaagctgacatcatagaagctattccatttaaaccgggaacagttgacatcattcgcttcattgaacgaacagccgcgtgaaacggagtatacgctgccttttcttcaggggttgatggtcgtttaagcacgctgcctttttcgtcaatgatttttgcttcatatgcgttccattcagtgaatggtttttgcattagacgaataaacttatatgcataaacagcatctataccgtttttaatagtgctcataaacacctcctacgatatttaatcaataacaaaaacggctttatttccgtttgcgtctgttacttcataagtatcttcgtcatagagaggtataaaccattctacatcagtatctgacataacagaaataatattttctgaaatttttgtcatttcgtattcttcaaataaacgaaacacatcgatatttgtacttgtgcaagttgcagtagacatttagcctctcatttaaagtccaatccattccagcacttcaggaactgatttattttcacaataaagattagtgccagcaactaccaaatcattctggtcgacatcttcaatgagcatcttttgattcgcattagctccagcaagaaggtctttaattagcttaggttttaccgtatcccaatcaccttcgacaaatttaaggcattgaatagaattataatcagctactcccggagttgcatgggctggaactgctgaacatgatgctactaccaatacgagacttgcaattaactttttcattattactctccagtttgttgataaagtaatagtatcacatccttccatggatgtaaacggtcaactcaaacttttcattgcttcttcaaaagttaattttttaacggaaagataccgcgccgagctaattttcttaccattgctaaggacgaccagtgtcccgtagtcccatcctgataaatctattgagttccatatatcaacacatacttgtagtgactcatccgctactatgaacttctcgtgaccatctcggtcacgattggcaattaaaacataaatcaaaatttaagttcctctgcaagtgcgtccagcttagctcgggctgatgtctggtctgtgcgttgttgtctattagcctcggcttttcgttgctgagctccggatgtttcagtaactggagttggctgagttccaccttcctgtgcaatttcaacccagcgctgatttcctttcttaacgcctaccgcaaacttattccaaatatttttatcgccataacgagatttaatctgcttgatgagttgttgtcccatctgagcaagttcttctgtctcaatcacagcaagcatgaagtccgccgttgccggtaagccagctgattctgcaatgtcggacatattcatatcagaagcatcccatgcaccacgaccaacctgagccgcagaccagactacagtttctgattctactgccaatgcacggagttcttcggcaattgctttcactaaagtgtaactgttttcagtataaacacgaatacgacaagaaccacatattcctagatagtcaatcataatgatatcaggaacaaaattctttttaagttttaactcatttagtaaagctctaaatgtgttcgcattagctccgccggtaggatattgtttaatgattaaacgaccaagagtatttttatctcgccatttttccatcttacctttgtattctgcatatgacacattgccatcatcaatatcatccaaagatacatcaagcaggttagcatcaatacgtttagcgcagacctcctcggccatctccatggagatataaagaacattcttaccagtttgaagataatcagctgctaaagaacacaatccaagagacttaccaacgttaacaccagccatgagaatgttgagagtgccagtttctgccccgcccttagtgatacggttcaaaatattcatcaagaatggaactttacgagccttgttctgatacgctaaccaacgagcttcataatcttccatccaatcatgaccgacataagagtcaaaactaatagaaagagcttgacgcataatgtctggaattgctccaacatccggtaattttttatttcttttttctggcggtagttcagcgttcgtttgaatttcaatgatttttgatgttgcgttatacatcgcatgagactgtacatatttctcagtctcttttaccaaccatgcttggtcttctggcgtatctgctaatttatcaattaattttttagcttgttcagcttcagattcgcctagagttgaattgtctaatgcaatattcaaagcattgattgaagggacagattgatattcgttgatatgtttttgtaataagttaaacacatttttagccgggccatgttcaaaatattctgaatccatatatggccaaacttttgcgaaatatccttgattaaaaataagatgcgataatattgtttctaccacggtaacctcattaaaataatttgaaacgttttttattacgctcagctaacgcttgctcaatttgaattttaacacatttttcaacatgcggagctagttctgcttttctatcttcggataatgtagaaaaatctacagaaatttcgtttccattgtgatttattgatgtgacataaactatatgtgaagaaccatcttctaatgttagcaaaatttcttgtactatattagccattgcctttttgacaatatctaatgatttttggtaaactctttctgtcctctcaaattccccctcctgagagggggcttcttcgacgacttcaagatttaaatctaattcgcttaaatcataatcgtcttgtttcattattcgtccatcatagagtcaaggtcatcttcaattgctgccggagacggagcatgagaaccttcgggagctttgaaaacctcagtttttgaattaatcaatgcatcaacttcagcatcaacaaccgcattactatcaattgcgcccagttgatatttacgtttaattgcatcacggaatggttgatgcttgaacatcggtccccagaaatctacgcacgatgtagctgctgcgcgccatgatttttcttcacgaaccatttcaccggtttcaatgtccaaatattcgcgagcataccaaccattttttggtttaactacaaacccgagctccaatgccatttcaagcaaaccagaatacgggtcaataccaccgtcaaatttaacatcaataaagaacttagatttctctttaactgtacgagatttttctgcgtttaaaacaaactgataaccttgcaaatcagtaccgtctttaatctgacgtttaccaataataaacacggtatccgcagaatacatcggaccagtaccaccggtcattaccgttttactgaacatttcaatagtttcaattgtatggttaactgcaacacatggaatgttcttaatactgaagtatggagtaacgatacggaacagagatttcaatgctttcgcacgggtcatatctgcgaccgatttttcgttcaacgcatcttcagtttctttcttggaagccaggttaccaattgagtcgatgaatacaataaccttttcaccacgctcaatttcttctagctgatttaccatatcaatcttaagttgttcaacagactgaacaggagtatgaataacacggtcagggtcaacgcccatagatttcagataagccggagtaataccgaattcactatcatagaacagacaaattgcatcaggatgttttgtcatataagcactgaccattgtcagagacatattcgatttaaagtgctttgacggaccagcaaagattgttaaaccagactgcataccaccatctaatgccccactgattgcaatgttcagcatcggaattttagtacggataacgtctttttcgttgaagaattttgacttagttagttccgcggtcattttagaagtggatgctttaatcagacgagattttaaatcagacattcattttttccataagtctccattatatttttctcataggtttaaaggatagagtaattatatcaaaacaaatttaaagcgatttaacttttattacgcacttaattggtgagttgaccattgattttcgaggtttagacattttaacctcaggcacatcctcagttcttttaatccaacttttgtatggacagtatttttctttcttggtcatgatatttccggtttaaatttttgaaagaaggtacactagaagtataccttcgcgaaaatttttaaacttacaggtcaatagccttgcggaactcttcttgccaaattggtttttcatcaagatatttttgaagaattttgtgctgatatttcaaagtttcaacacggaactcttcatcttctttaatcttgttgattttatctactaattcttgacgattactcacataaaagaaatcattaccttccatgatattcatgtcagggtcaaacgtgctatcgaagaatgccactgcagtagatgctagagcttcccatacacgaggagtgatttggttgttatcgtaggttttatcacctaatacaatagtagcatacgcggttgagttgcgttgaaccatttcacgagagtctactttaccggggaataccggcggagttgtccaaggaaattctgggttcttgaactgttctgctttcactgaaccaaagaattcaacatccaatccagtatcaaataggtattcaatcattttagcttcacggttaccagaacggaaagtgccgccataaatcaggtctcgcattttgatgccatctgcggcaatcttaaagacactatgatacattttatgacggtccaaagcaaaatgaacgaattctagtttatcggataaacatccaactaaacgatcagaatgaattttcttgcattgctctagatttcttccttgagatacaatacgcattggagatgttacaatgaattgttcttctttatatttactggaccatttctttttagacatccttcgccaagcttgctcaaacggaagacgaatatcggtgaacaaataataaatttttgatttatacttattcatgaacatataagcttctttgttcatcttattttcttcgccgccatagaagttcagagcagcattaacgaccaataagcggtcatacacattcgggtcttttactgaatcgaaagaaattccatactgtgtatttttcattgaaataaggtcaacatcaagacccatatcttttaaacattcggacaaataaattgtttctgaagcaggggttgttttaaacccctggatgttattgcccatattaataatagcaattttcatagtgtcggttcaatctcgctaaatttatgaaaagcatctgacttctgctttttagcaacacacatacgaagtactttcaaaggttcatcagtaccaaacatagttttggttgggtcaccttctgatttccaccgagcttgagttgggaagtcagcatgaattttttccagcgctctgttctgtttagcagcattacgcatagatgatacaccgcctggagcttgtcctttacctgatttaacgagatatttaaaaatcgccaaatgaggataacccatattaatgagtttaagaaacgcgtatgtatcttcggataggtctactataccatatccaatgtcatcggcagaaagtttgcttaaatcataaaaagtatttgtaaacccgtaagagttctcacggaaatgaccccatttagagtcaatcttgaatattggcaagcgagcgtgaccatgataaaagccgcagtccatcgctgtttcaacataaagacaaagattattaaattcatcccaggtcattccaacatcatggagaatgcgccggtcatctttttcgcgaatttccgttgtatgaatagtagtatcatcatccaacatccagatgcgttgtccttgatacatttcggtaattaaacgacgagtaccagcaatcccatttacatcatccgggatagttactattttagcaattgtgctatatcgagtttcgtattcttctttttgagattcgcgaactacaagatgagcgacataacctttagggaacatgtctagggcagtaactgccccgacacgattataacttggaattacaaattgaatcatttccaataacctttgtaatctttctttgatatatgagtttcaccagtatggtaaaaatggtcaactagatagaagtgacgagaataaacatgcaaactacctacgttccaaataatgtcaccaactacgtattttttatcgcggccacagttaagtgtttgtactaataattctaacacatgtttttgccaagcatagtcatttctaaaaccgaagactacatcattgcttctcatactcacaacagcatgaacttgctcatcgcgaattaaatattgaaccgtgtttgtacacatgaaatcactcataccgtcacggttataatcagtctgcatacttggacgagtataaatcataattccgcggcgagaatctggattgacactcagttcagccacacaacttgcaaattggttataattatctttattccaaattgcccatccgtagtttgagttaatctcgccgtttttggatgaaatctggtcccaaattgctggagtttttcctggaatgtctttaacgaataaactttgagacttataccattctaattcgcgggctacatattcatcattaactgaaccaaaaataagttcttcatctgcaataaaagatgcgccgataatttcaatagtttttacgccagttttgtcaataacaaactcttcattttcgagagctacagcaaattcctgacgaatgtcttgtactgtcaaaggtgtgataatcattttgcctctttaattacgataatttgttgaggttggttattttttcgctcggcgacgaatgcccaaattaaagcaaccaaccatccaatgaaagtccaaccaaaaataagatttaaaaagaaaattccaacattagattttgtcccacgaagaagagcaataatccacggaagaaaatacgctaaaaacgctaagaacaatgaaccaaaaccaagtgccgctgtacccaatacgatagtttccattataatttcctctattttatcaaatgatttatttagcgtttttagccatacatttattaaaagctttcacttttccgcctgaacgataatgaataactgccagacgagttttattatttggatttttgaagtattgttcacgaagaacttcttcatgagcttctacaacaaattcacacattctgatagctttttcctttttatcgagtgcagcttgacgttcatcgccttcagctactgattttttaattgcagcttcaagcggactattcgcgcgccgttcttcaatttctttaatagcgttaagagtttcctgcgagacttgagaagttgcacgagtataaaccattccagaagctggcttataaaccaaattaccttcacttccataatgtggttgcgaataaggttgaatagaacaacctgctaaagtagttgctgctagaacaatcaatgatttcaaattaaatttcattttattttcctcatttgttttgatgagataatagtaacacatccttccatggatgtaaacggttagaagtcaaacatatcttctaatgatgctttctcttcgtagtccataccagcagcctcacacatgccagtcaaaggtttgacaaaagacttctgaaagagcgtagtgtaatccatccaagccaatacttctgaacgaatttcctgtggcaactcagtaccggatggccaagcaatacacttatcaccaaacgggtttccttcacgcaaaggaacaatcattactttattcccctcaagaattggagtagcgctgaacccagcagtcgctcggttgtaagtcaaagcacctcgaatgtggaacggacacttaaatccaggccatccgttatcatcatacttggcgatgtcattacatgtcttaacttctgcaatgattttgtaatcaagaccacggtattctttttcaaactgtttgtaatattcttgtactgatttttcaccttcttgaagcatacgacgaatactttcttccaatgctgcctgcaccgcctttggagtagaagattgctgagtttccatacccatgatttttagatgcggctcagtgtaacgagtatcttccatgtcatatacgttcaatgcataacgtttcttagctttccagaaaccgccaataccctttgaaccaagaggaggacaagaaatagcctcacggtccatatgcattaagtgttctttattattcatgtactcgcaaagctcacggtaagctttgtcaatcataggttccatttttttcttaccaaattggttcatgaattcaaccaaatcattggtatttttaaaacgctctaaacctactttttcaattactttatcaacagaaacgtaaactgagtcagtgtcgcctgccgctataaaatcttcatttgttgtaccgcatactcggttaagatactcgttaatcttacgagcaatccactggataccaacctgaccgaacagagtgatagcagtcgcattacgaaggtcaaaatatcggaaataaatgttaccgagagcaccataaagactgttgatgagaattttacggttcaactggtttgtattagccaaaatactcgcaagttcacaatcatgcagcatcgctttcagagctgacacacttaacctgtagagttcattcttcacttcatcgctaaaatctttataacgctcaacctgaattttaccgccagttccaccggattttgaagcgataacagcattaataacttcgatgttacggttttcggcttgctgctttttcttccaatctttacgttggaaaaataccttcgcaatctcaaccgggataataccttcacggtctttgtgatacatccaaccatttggtgaacatgaatatgtgtcactaggacgcggtgctgttccattgatgtattcatgaattggatgaagcttaaactgaccagcgatagtttctggactgatattcacttgacgaatgatacttggataaagagacgtcaagtcaaaactcatgatataattacgagcacatgcttttggttcaaatacgtaagcacccgggaagctctgcttaacatgcgaacgtccttgcggaaccactcgcttatcttctttgagactattgaaaataatagcatcccaggttttaatcgggctcattacaccgccaaaaggcattttagcgtaataagacatactaattgctaggtcgatgaacccacgaacttggtcgataccttgcaccgattcaacgtccatgatgttataactaatataacgttgatggttcgtttcacgcaatttgttaatcggaccatcataaggcagtttaccttttttggtctcatattttgcgacataatccaaagtataagacggttggttcgtgaaactgtactttttgtacaaatccatgtaatcaagaatagttacgccatcaatgctaaatacttctttatcaccatacatgttagtgataattttagatttcacgcgattgattggtgaaaaacgcttcatcgaacgttcacctagaacttgcttaacgcggttcatgatatacggaatatcaaaaccttcaatgttccaaccggtgaaaattgctggacgcttttgttcccaaagacgaatatattccatcaatagttcggcttcagtttcaaacggtagataaacaacccggtcaagaatatcttgcggaacttcatcgccgccgtctgcttcgcaccgagctgctaactttttatcccatttggatactgacccatacaacgaatggagtaaatcaaagacataaaatttgtcatcaatggaatcgtaatgagtgatagcatcaatttcatattctgctttcatcgggtctgggaatttatcgccagtaacttcgatgtcacagttcgctacacgaatgaattttcggtcataaacaatttcggaaccatatgtgtcactgatatatgcgagtttaaaatcatccattcccattgcttcaagaccgacatcttccatgcgcttgaaccaatcacgggcatccttcattgtaggaaaattaattggttcgcagtcctttccatagatgtctttgaattttgatggtttatcaacatgacggaacagagtcggagaatattcgacgcgccgagttttttctacgccatcttcaatatatcgttcaacaatatcattaccaacagtttctacagatacataaaatttcataaggttccttagtttttccagtggtccgagtgattagaccttgggtgtattatattctattctttgaatagcagaaaaggcccggaggccttttcattgagttacgcgcctatagtatatttcgcgataagtttccaatcgtctttttgcttaaatgtgatgacgcggaaattattttgcatagcttgcagagaacctctgtctacgagttcgattaaaccccaatcttccaggagttgagcaattgagtcgcggcggataatatcttcatcatcaatagtgactggccgtccgtccagtttaagcatctctttaaaatgaactacataataaagaccctgcttctgtaagatatgacaactttgatataatacacggtctctgttattagcaatacccatacgagtaagagtttcttttactttcaggaagtcttcaggttgtttcagtttaatttcgatcatttttaccattccaatgcttgttttttaaattgtttttgttctttaacatttttggtcacgtctttcaaaaattcatctgtgaccaaccctttggcttcttttaatactacgcccaatttacctttgcgttctagggtttctctataaacctgagcgtcatttaaattaatagtataatatttcattaataccctgagaataagtatctcctgggtatcctctattaacttagcccacttaccaaaacggcggccttgaggaacagcagctaccatatagttgaaatgtgcttcatctgacagttcagaaccgataagattcattgtataaactgctgtcatgcattccggatgttgcgataacgcattttcaaccatgaatttgttatattctttttgagcgatggaacattgtttcttttcgttaattgcgccgattatttcaaaaaattcattttctgctttctgtttgaaagtatcagccatttcttgaactttgttccaatctttagaataccaagctacctgatgctcattaaattggacgtcatcatcaaataaactcattatttccactcattattaagttcagctgttaaacgcaagaacatatacatcacatgaagttcagtatttgaggcaactcctttgtactggttgttttcaccaataatttcgtacattgccattataccaggtcctttaagcatagagtataattcttcggcgagtttacctacaaaccaagagtaatcagtagaatattttggagccaaagccctaagctgtttaacatctcgattttttagagcatcaatcacatctgaaattgaactagagtctttagttacaatactcagaattccagcatccagaacgcctttggacgaatacatatcaagttcgccaatagtcttacgaaaatctggaaagttctttttaactaaagcagcaacaacttttaaatcggcgatttcaatgttttcatttttacatatttcaaccatacgacgaatcatctgtttcatcattgttgtacggtcttcttctgttgcttggccaaatttaattactcggcaacgcgaacgaagtggttcaataatgccgtcaatattgttggcagtaataataacagaacagttacttgaatatgcttctaaaaatgaacgcaaatgacgctgagattcagcaagtccagaacggtcaaattcgtcaataacaataactttttgacgaccttcaatagaagcagaacttgcaaaattagttaatggcccacggacaaaatcaattttacaatctgaaccattcacaaacatcatatcagcatttgtgtcattacacaaagcttttgctgtagttgttttacctgtacctggtgaagcagataccagaatcatatgagggattttaccgcttttaacaattttattcattgtttcgcggtcggcggcgggaaggatacattcttcaattgtagaaggacgataccgttgttcaaggatgtgttctttttcgttaattgtaatcataatttcctcatattaatattcaaagcaatggggccgtaaccccattttaaatcattttatttacagcattaaaaatcatgagtagaatcagcttccatagctactacataacttgcatgttcaccttcaaacttagctgctgttttcttaccatcagcccacagcatcaatttgtatgaagcgggttgcattttcatattagccatgttaataacaaagttaaagttattggtgccgtcatagtcacccaaagtcaaagaatattttacacgaaccaaagcagaatcttcaactttattataaccattcagaataattttaccatctttgttagtgaatgtgatggtatcaatctgcaaaccgcgagaaactcgcatcagctgctgaaggtcttcacctttaaaatctacaataactgaagcgaccgggaacggaattggtttgctcggaaaaacaatggtgcttgggtcagctgctggccagaaaatagttgaacgagcatctgcgattttcacattaccatcatctgccatggaaatctctgcgtcttcattgaccaaaccgagaatacccagaaaaccattcaaatcataaattgctacttcaaaatcaattgtgtctgcgatagttgcttcagcataagttgtgccgtttacagcacgtgtcatgataaagttaccctgcttaagcatgataccagagttaatagtagagaaatttttcagaatattcagagtatctttagacagtttcattatttttcctttcaattacacgtttaataagatttattatattacttaaaagtttttgcaagttcttcgataacttcttcagataccagcaaagcagcagaaatcaattgttcatcagcgttaacacagaaatcatcttcaagacgctcgccaccaatacggtccagaacaaatttagcgccggcagaaacagattcattgtaatgcttccgcatcagttcaatccatttatcatttacggtgcggtcaattgactgataatatgcatggcgcgcttcagcaatagggatacgtttgcggataagttcagcaccttgttcacccatagcaagtacccatgctcgacgaattttgttttggttattagggttagagtcagtacgtacatttttaggctggatatctttagtatcaattgcaaaattcaagttcataatatttcctattcaattaatcaaagtattctattactgtccaagcggtgttgcggttagacattattttaacatattcgtctttaaaagcatctctgagctgtatttcagtaactaagtggtcttcacctgaaaaattattagtcagtatatatcttttaatttccggtacggccaacagctgttggccatttttaaagtctgtcatttttccattaccgtgaatctgccaactttcttcatctgaagatgttgtccataatcctgcgggtcatggtctttatggcttatgataaagatattactatcagtcattccattgagaattgttcctactcgcttaataccttcattatcaaacgcgccatcataaacttcatcaagaaagagacagttaattttaacattagaaactttttcagcaatgtctcgccacgtgaacagcaacgcaatatcaatacgagctttttcgccttggctgaatgacgcataactgaattcttcgcgcccgcgagatttaatagtttcggtgaattcttcgttcaaagtaaagacataatcagcttccataattttaagatactgattaatctgtttgttgaacatcggaatatatttgctaataatagctcccttgatacccgaatctttcagcatttcagttaaaatgcctcgatgatatttttccataaccatattggatttggtatcaactattttattcaattcttcgttaagcgttttaatctcatctgcatgattgatgaattcttttgatgctttttcaatcgccgcttttacttttttagccttttctactgttgaaattaaagtttgctttttagattttatgtcttgagcaagttcacgttgcgttctaacgttgctctcatattcatatactagtgtctctagattggctctatgactttctaacgactctgatttatgtttacattcattaatcttatcgttaatctgagtgactagagatgacccagagtctaattgttgcatgcatgttgggcatgaacctccagaatgatacaaagaaacaaccttattatatgagtcaattttagacttaatcaacagagtttgattaccaatttcagtaaacgcggaccgagggtcttcatcaagcacgatagatgttaaacgctcagttgctttttcaatttcggatttaatagaacgtgcttcagtcgcaagttcatcatacatggtctgaaaacgagctatattttctcctgatagctttttctggcgttcaacgttctcttcataaattttaatctgttggataattccgtctttctttgcgtccaacacagagacttgtgaattaatttctcttatatgagatttattcaatttatccatttcagctaatgtagaaacttcgagcaagtcttctactaacttccggcgagctggagtactcaaacccatgaacggtgtataaccagctgtaccaagcacaactacttgcttaaagctggaataagacattttgataagttcttcaaaatagctttgaaaatctttaacagaagctgcctcgtccaggcgaactccgtctcgagttatttcaaagatattcggcttttgaccgcgctttatataaaaatgcttaccgtcatactccatccaaagctctactaacatatcttttttattaatagagtttattagttgacctttcttaatatcacggaacggtttgccaaataaagcaaatgtaatagcttccagcatggtactcttgccaccgccgtttttgccggtgatgagagttttttgtaccttatcaagttgaatatcaatgggcgaaccgcccactgacataatgttctggtacttgatgcggtttaacttaaatgttttcatgtgttattctcgcgaatataattgcggcaatattccagcataaaatcttcggatatatgcattacatcaaaatattcttgccatgatacacctaatgaacactcagatgtaacaaacattatgcctttaaacccgccgtcttcgggccagtattcaatattaccataccaaccattgcctaaattaaattcatgaagtttcacgagttttgaacctcgatataaagttgattagcatattttataactgcttccctgtcatcatcggataaatcaggtaaagagttaatatattcttccataagttcaagtaagcttttaacttctacttcagcttcttcatcaacttcaagagaattatcaatctttgaaacggtacgaagctcatgaacaactttttcgagttcgctttcaaattttggaaggtctttatcaacttctgtgataatgacacgaactgaaatatttttgtaacgattaatatcaaatttacccttaaatggataaattattttttgatgccagcatgtttcgttttcgataaaatcaaaactgtcatctcgagtgtcctgtatccagaaacctcgcgggtcattttcgtctcccgcagtaagtgtccatggcgtgccaatatatttgacattagctgcgctagagatagtatgaaagtgtccagaacgaacatgtttatattttttaaggaaatcaggttccaacccgtgggattttagtcctttgtaataataaaaaccacttaattcccaatgaccaatacaaatatcggcatcagatgttttgatgtgattcattatatcggacgcgttctcttcgcacatccacggaatcatatcaatcaaacaaccatcaaaatctacagtttcaggagtatcatgaattatgatatggtcatatttacctaataactcagtgattgcattcggatgcattttgtttttatagtgaagatcgtggttacctacgatggtgtgcatcgtaataccagcttcacctaaagaagttgctatctcacgagcaaattccatagtcttgtgagtgatagctttacgaacgtcaaaaatatcaccatattgaacccacatagttatcccatgctttttggagtattcaatcttttggcgtattccgtctcgttgaatattctggagccatgggtcatcgcccttaactcctaaatgccagtcacctgtatggagaattttcatattttaacctgtacaaaaatgataattaattataccatgtatataaaaagcaaattagtacgcgcacaatatttttgaaacgtttgactggttaattttgtgaataaactcacatttttcttctggcgataattcgtgatttctgatgagtgccgcgcaaggatattttggtggataatctttctttaaaactaaaagtaactcatctaaatatccgcaactatcataagcatgccgaagtttaacaatatcaacatgcttaatgcgttcaaaatctttagagctttgtactgtacttcgccaatgcacttcattatctttagtcgttggagaaatataaactgcaactcttagttcagtgtcatttaatttaattttttccattattacctcaaagaacaaagggagcctcggctcccttaaattttacgatatgttatagtattttcagcttttgctttatcgtgtaacagtttgaattgtgacatcattccagacatgctgttatgtatttttgtataatcaacagtcttctttggagtttctgtcacgatttttaccatttccaccaccgtttaacttgaaaagtatcagcccaaactaatgttttttcatctatagcaatctctgaacttttagttacaccatacggaccaagaattttgtatgtgtttttagaagtttttagagccattgttccattaaccatagatagagtgaaacaagcattaactttaagacgtttgaatttaacctttactattgccatttacaatttcccaaatctgtcgacgagtggtttcccactgtactttgacaagttcatctgtataaggttggcgtaaaatgtcccgagatttctcgatagcatatttgtacgccgcgaagttattttctagtactgctctttgcgcatgctcatttaaacgtttaagttcttttgcatgctttttataaattttcgctgctttagcgttagcttcttgacgtgttgcttcttccattcgagcgataatttgttctacttcttctgggttttcatcaattttattgatattatcaataatctctggagacatttaattaccttattttggaaaataatccaatgaaaaataaaatccgtcgggagatacaacatattgatgaggaacttcttttccatcaacaataaatgtaatatcgtgcggattagttgggtcaatatgaatatccatttcaagaacttcccttaaatacatttttagtatataaggaatagcttctaattctggaatatcttcaagcattcctgcgaggtcaatcttcagcctcatataaaaaatccaaattcggagaatcgtctacttcaacactctttttgtcagaccctggtgctttgtaggtagattcttcataatgcgtcattttatcgtagatgtcctgaatgaacgtttcatctgctaacgcgaccatatcatcgtcacgactgtcatagacattgtgaacaaaatagctatatttctttgccatctctttgcgttcttttttgatacgctgaacgaaagcattaaaacaagcctgagttatgtatgcatgtgggtttttgtatttctcttcgtcaaagtttttgagacctttaatagcagcctcaactccatcagaaatcatttcatctttccatgattgggtgtatcctgaaaagttaaaacgctttgacaatccctcggaaataagcataatagcttttccaatagtgtcattctgacggacaagcgttccttctggcgcttctctacatttttgtttccattcacatattgcgttgtaaagctctttgttgtttacatagtcagccattaatacctcatcgcctcaagtttatatatacttttattatatcatatttgtgggacaagaggttatttgctaattagaagaacgaagaatgcaatacatctgaaaaagttgttcaccaatttctttaaattggtcaaataaaagaaaataagctgtgtatgaatacaagtcattatcttcccaaaaatcgttgaaactattaaacatttcggtataatctttttggttgagttttgaagcatgagaatgccaatatactccaggcatcacaccagtttcaaatttaattcccatcatataatcaacactaaaaataccaaaattctcaataattaatttaagttctgttcttttcattgctcctcacatgtttcggcagttaacttcaaattatcagatttttagcagcttctaaagctttatccattgaactgaatgcatctaccgagtgataatctttacctgtgtattcatataaccaccaggcgtcaaagtcttcttcaataacgtaaccaacaccgttcttcgaagtaccttcaatcatcagcctcccatcttcaacagaagcatcaaatcccgcgagttctaaatctgcaacaatttgttctttattcattcagatattcctcaagctcttttcttgttaatccttcattccacatagtagaaggattaggacagtcagtgttgctagtactaataggataatatctcatttctttaccgagagcatacagacatccaaatgcttcgtaataattaacagcttgttgagtttcaatccgttcgggcaatgaaatagcctttgcgcctgtaatagtgggtttcaagtaaactgcaataagtgctatgccttcgccgaattcgctcatttcgccgccttaacaattttgaggtcaacaaatcctttcttgcgctgatttttcattttacgaatagtgcgttcggaaatttcaggttttttatttgttggaataccaaaagcatctagattaaattcatcaataatatagcaaacaatcaattcgcgaattcgagcttttccaatcttctggtcgttctctttcatagttccatgaagagcagtttcccattcatctaaaatttcaggcgtgacaataacagatttgaattcaccatcaactttcatttcttcaactttagggaaaacaaaagagttcagaacgtttttgatagcttgcgacatgttattttcctcattggtcgttagatgtttgacatagagattttaaattaaaatccctatagcaaactcaactcatctgtatgtttttagcattatgaacaattttgaaattatccttcgacataccaacctttatcttgccacgcttcgcctttataccaacgagtcatatcataaccgccattgtcgcgaaccgttccattatcataaatgaaatactcatctgatgtgaaatattcatgacgaatacacttgccggtaatttttgaagtcatcacagcttcttggtaagtcattatgcacctcttacagcatcaatcaattctcgtaccacagtatccaattcacttgtgtatacatccggaaccagagctttaatacaattactcattcgtcccgggtagtgttgagcatgattgagcgaaacatctgtccgacaacccttcgcccggaaaacaacttcttcgtcagagatttcaactgtgagtggcactttatcggtggatgaagtacgaataatcatttcatacttaatcatactcgttttagatacgataatcatctttaactccatttgtttggtatgaggtaatagtaacacagtttttggaggatgtaaacggttgatatgaaagaaaagagggaccgctagggtccctcggatttaaagtcaatatattatattagaatgatacaagaatgggatagagtatcactcttcgtccaggttataacctaagacgctaagttgttctaataggtgggatttgttgcgtattctttggttgttcaaaaagacgataggatatcgtagagctaaactcggataactacctacacgtttagccgcttcagtaattcggtctctgtcatagacaaatccaacttcgttttcgtttggaattattacatcgtaaaaagcataagggatattttcatcatccaaagttttacgaattgaatagcaggccggacaccggcctactgtctcaggaattccgtatatttctatttttgcagacatcttatttcttcaataagcgcttcaatttgtttaagtttatcttcaattgaagctttggaggttgattcaattatgtatttctcgtcaacttcatatagaacatctttgtcgtagaagttgaataaacagctttggtcaaaatcgtccgcgtcaaaaatacgcttatcatgactaagaattctcatgacttcagcatgttcaccatacccatttaattctagtactttaaatgcgccgcctttgagcttgtcataaagtttttcatcaccttttgcccgtcgcgcagataaccatttgcttacttctgccgggttataagcgtaccattgacctgctttgaattcagtaatcatattaatattgtccaaagaaaatgaaagcataaagagcagggaactcttcactaaaacgctcaatgtaactgtacatgtacacttgaagttcatgaatccaaggatagtgaatctgcatttaaccattcctcattgtgttgtttccatagccgctggttatctgaaccacgccatggcttatcagttgggttatcttgttcatatttaccatcaataataacatcaatgtattttagcaattcaaggtgcttgatgtcttcaaacttgtaaccagtccacatccagatatcttttttgggaaaacgcgctttaacccatttaactagagcttcaatatcttctctgtttcctcgataaagagggtctcctccagtaagagtaagtccttggatgtatggttttgaaagatgttctgcgatttcttttactgtattagtgttaaatgtctcaccgttgcaagggttccaagtgcttttattatagcatccttggcatttatgaatacaacccgtaacgaaaagaacgaccctgcaaccagggccattcacaaaatcgcatggataaattctatcatatttcaatgtgttttgtcctatgcatgatttctttattcttacctaggttaaatcctcgatcagaaggattaccgagatatccgcaagtccttctaatagtattcatcttcttaggattagtctcgccacaaacagtacattcaaatccgttttcagttggtttcatttcatgagttgacccgcaactcaaacatttatcaactggcatattaacaccaaaataatccagtttttctacagcataatcccagactgcttcaagagcttgaatatttctcttcatatcaggcaattcaacataagatatgtgtccaccctttgcaatgaagtggtattttgcttctcggtcaattttttcgaatggagaaattttttcttcaactgaaacatggaagctattagtataccatcctttgtcggttacacctttgactattccatgttcggctgcgtcaattttacagaaacgataacataacgattctgccggggtactataaaggctaaaagcatatcctgtttcttctgtccattcgcgtagataatcattcatgtgtttaagcactaattcaccgatgcctttgtgggtcattagttcaagctcgtgaattccgatatatccaagcgaaatagaacttctaccatttttaaacaactcaatgatttcatcatctggtttcattcgaacgccaaaagctccttcttgatacagaatcggagctacagaagcttttacaccttttaacgattcaatacgaagcagcaacgcgtctttacaaatcttcattcggcagtctagtagacgcatgaacatgtttaaatctgttttgccgttgacctgagaatccaaagctatacgaggaagatttaatgtaacaacacctaaattattgcgtccgtcaagaataaatttaccatcggaatttttccatgcgctcaaaaagcttctacaccccattggagatacaggaataggagaaccagtaatcttacgattatttttggcgctaataatatcgggatacattcgcttagaagcgcattctaaagctagttgtttgatatcataatttgggtcttcactatgaagatttaaaccttcttcaacgaacataaccaatttaggaaaaattggagtaattccatgacgacctaatcctcgaatacgatttttaagaatagcttgttgaattagtttttcttcccaagatgtccccataccaaatgtaatggtaacgaaaggtgtttggccgttagaactgaaaagagtattaacttcatattcatatccttgaaaagcatcgaatacatctttttctgttttttcttgagcgtataattcagcttctggaatatgatatcggcgagcgtcttcgatatgtttgttgaaagttgcttctacataaggagctagtacttggtcgacattagcaaatgtagttccaccatattgatgagaagctacttgagctgtaatttgagccattacggcagttgctacaccaattgaatttgatgtttcaatttcagcattaccgagtttaaacccattttgaagcattccggctaaatctactaagcaacagttagtaaacggaagcgatggtgaataatccatgtcatgaaaatggataattcctttatcatgagcatctagtatagagactggaatgaccttacgggcgacatgcttagaaacaatgccggccataaggtcacgttgggtgggaaatacccgcgaatctttattcgcgttttcatttaaaaggtctgtattagttctatcaaacagacctttgatttcagtttcaattgtcattttaagcctttctttaaataatagacataatcttcaagccattctattgtcaatagaatttcttcggggctttttaaattagcctttatttctttaaaagaatttgtcattattttagaagtatgttcaattttaatagtttctactaaatttaaagtcgaaagtctctcaatgtaagactttttatcttcaatttgtaatttaataatctcgccagatttaacaaatttagaacagcttccacgattagaaccaaagaattcttcgacttcggaatttgacgcaaactttttaccagtttccacatgttgaaacttccatcgctgacgtcgaattgaaggatgtagtgtccatcgatcttttataatatcaagttttccatttaataaatttgtaatatgagacgatgaaatatcattttttcttgcaaattcagcttgatttattacttcgatttcgcacgaatttagttcatcccaaattttaaatttagaaacagaatgatacactgataatttttcttcctttgagcgaccaaattgccagccagaatctaaatacttttgaacatcatcgggtttaacatatttggttttttgaccaattttatacatcatcggccatgaaacatgctcatttcctttgtcaaaattcatccatcgagcaatatcttcttctgttaaatcatcgaagcagttagggttaaaatgcccgccaatttgtttgtttaaccacatagggtctcgtaatacgtcattagcaagttgcatttccatttcagccattaaagttttaccccaagttggataccatccaacaattagatattctttaggctttataccattttccaacatcgaattcaattcatttgatgaagatgtataagtcttccaatctgattcttcaaatccttttttaggaccgcgtttaaacgttgaaggggcagctttaattcttttccaaatctttttagcgcctacatactttttaccgtcttcaaacgtgattacataaacaaatccaatattatttttaatatctgtttcttcaaaaacagaccaatgaccataatctgacattataaatacatcctttttactaatattatgatgtatttatacaataagctaaaccattaatttatctaaattatagagccaatacgtccttgcccatttcttacggtttcggatttgagctttagtcggcttagtagagaaccaccaagaaattttatacccatcatcttcaataatttcaaaaactttattcactaaactcatattaaattccatcactttttagtattcggagtatcaaataatgatatgctacaatccggatttccagttatatgacaacttgtaaatagagcccagcggctcaaagcatgtttacctaaaatttttctagacctgttattagattcttcattactgagagtcatattaagtcttggaaaataaacagaagttataccagttatttctccggtgtcagtaaaactcgtatagcatttggaaataacaatttcatcgccagggataatacccatatctttgaaatagtctatcacatcaccaagctcatgcttaactttaaaaacttccggttttactgtaagtttaagtccagttaaattcattataatttactcaattaaaaattaaacaagcgattaattcttttataccgacgaattaatcgcggagtcaacttagaaacgctcaaaaccgtcgtaaatgttccgtctttgttagatataacgaatttccatatgcaagaaccatctagctttttgtcatgaataattttagtattcattaaagccctcacaatctagcatctcgttcatcgtgggttcattataacatggttcttcatctgtgtaaactggttcttctggttcgggctctacggtttcccaccgagctgcccaccatggttttaaagcgtaacccattctgtgattgtcctcatcacgacgcgttcttgtttttcaacaagcgaaacatcaggttcattgtaataccaatccgaatgatatgaacccgaacgagtttcatttacagcaacatgaacatcatgctttttgctgtagtaaactacctgtcggtattgatatttgtggtcttgaacccagtcttcggcttccactttttcgagatattcagaatcgtcaaaatcataatattcgttatacccatcatggtcttccatgatttctttaagaatatcagtttgtccaggagtcattattcacctcgtaaatgattaagctgttcgtcatcaacccaactctcaagtccagatggcaggtcgcattcaatctgccaaagtaactcttcaacttcgtataaccgaaagagctcttcttgtttaataactgcttcgtcgtcattcaattcaatcgttttcattgtagtgtccacataaacttaaaaagattttcttggaccactgcgtcatcccaaccccagacattaatatcttctgctaattcagttggaagaacatcaagaacctgttgagcagtcggagctgatatacaaattcttttggcgcatcggctataagcagctaaaaactcgtcatagttattaatcataaagtttcaccatacaatcaccgtatttccattttgacataactttttcacccgtagagtaagttacttcaaccttagctcgatgattttttatttgaacaattcggccggtttcaagaccaccgtaaccataataaagcgcaattatatcattaacgaaaagctcctttccgcgataatcacgcatgtaatcttgaccttcaaccatgcgttctatcaccgaaaatactcgcgcaatttatcaaaaccaccaatataacttccatctggagcaaatatttgcggtacggtaagaccgatttgtgtatctcttccaagacgagtcaaaagttcagcaataatttcatcgtcaaatacacctttttctggcatgatattgatgaattcatatggttgttttttaacatccagaagacgtttagcgttatcacagaatacgcatttatgaatgttgctgtcgtaaccgaatactttgaacattattttacctctacagggaatacttgagctaattttgcggcaatagctttatcaataatttcgctaaaactttcgttcaatcgagaaacaatcatttcttcatattgtcgttcattaatcttaattgcatcagcgatataattttccattcgtttagctgctgcgtctaccgagctcacaagtgagtccaaaagttgttcatgcgctatacgttcagcttcttctttcatcactcgtttgagttcaactgtacccttaacattccagccattacgctcaaaaaacttctcgagctctttgcgaaccgcatcttctacgttaggaatgttagaaccctgaaaagcaatttcagatttaactgcttcagcaattttatttttggagtccttaacaacaagttctttaacaatattgttaatcacggattgttgtaaagcaactcgcattgcagagccttcagggaacaactgacgaactgcgtgagtatctaattttaaatcaatcattctgtaataacctcatagtatgcgtcgctcgccgctagataaattctgtcagatatatttttatacggatttttagtaaatttttgatttgctttatgtttaaaataaattacatcaaaagctgtagccgcgttaatacagttatgaataaaattaatatcagttttttcgccggcgtcaatagtgtgttgtacaataatttcagcgtgtaatttacgaagttctttaaaaagagacttcatttcctctgattcgcacattgcgtcagatgaactaaagggattcataactaacctcaatatcaaatttaccaatttcacgaaaaattaaattcaacaaatcctcttcagtaaaattggacgatgagcagcaactagcgaatggatgaagttcattatattgtacaccatcaataactacatcgtatccctcggctgaattccagcaacccagatcgcaatctccactatcagcatacagcttagttacttcaattttcatttagcacctttgtaaatttgtgtttacatttacggcatttcattttaagaactttagtatgccaattaattaattgaacttgattggtttcgcattttgggcaaggagtaacttgtttcatggaccgttcacgacggtccgccatttctaatacagtattccaatcaatttcaaaatcttcttcattttgaactaaatcttcttttaattggttccgcataatactgcattagcctctttaagtttctgtgattgttctttaagacgtttaagttttaaattagacttcttaattttatcacttaaatctttttgcactgatacttctttttcaatttccactttaattaggtcaataaagtgttgaatttgattagaaacatgagtttctaccaatacgtcagttgtctcaaactgcattgtcggagaaaatttattataaaccaccggattattagcgtcaatgcgccgtgaataagttccactaatagtatctttcatatcttcagtctctttcaatttgatattaattttaattcttgttttacgttttgaatcgccataattaatatcgacattaacatcttcaacataattcatatgctttaaaccagcagcaaattgttcagctataattctagaaacattatcagactgcccaatcatcattttaattgaaaattgaatatcataatctcttaaaggagacgtagttacttcagtttgaacatatctcaaccggttgataaaacactgagaatattcatgaattaaggttttacggccaagtgtaataatttgtttgtattcactctttaaagcattaataaatttgtatttaaaaatttcagacttttcacaaataatttttcgttcactcgttgaatttctttcttcagtgaacatattcaatgcacttaattccatgaatgagcgcaagcgcatcactgacgtgatatatgaagttattttacaacgagtcacttcttgaaaaaattcatcatatgttttattatacgttttaatagaataaaaattataattgaatttgttaaacagaaaattcaatgttttagcatccagaccttttgaaacaaaagcatcaatatattgctttgtccgagttttattttcataaccaaaatcaaacactggcttcatatattcttttaaatctgctgctgttttaatttcttcaaatttcatttcaattatctccagaatccgtaaacccattataacactattaattagtaagcattttaacagcagatgggcgtttataaaaggcgtgagtattgtcatcaactgctttagtaaaagtagctttgaactcaatttcgccgcgataatttacatcaactgctccgggtaatgaaccataaacagtagctccattttcaagacgcaccatcattttagcagatactccccaatagtcttggtaaatttttgttgaaactactctgccttttactacttgttttccttcaggtgcttcgcctttagttgctttaagtttatcgtagtaagcattgaaccattcttcggagtatttctgaattgcttcaagaatacttttatgggctctaatttctgccatcaaaactgcagtattatcttcaagttcccaatgcttatatggttcgcggacttcaatagggcatccgtaaagcttgagctcattaagcatatcagatgttaaacgaagtttccaccagccaaaatctccagtgaattccggcttatctaaaacatcgagctcagtaacatacggaaggtaactccctccatgataagcttcaacttcgccaagttcattttcccaaagataaccgtcaaacggagcatgtggacggtcgtcaattccccatgttggtgcaaccaccctttcaaatccttcgcgactgtagagagtcttcgcccatcctgcattcaattcctgagcgcgtttttcaactttcgctcggcgagcagcacgtgctttgcgattttcatctaagattgaattgatgatatcagttaacattttagtctccgttagtagattacgaaactatcatatactaaacttaatgcgttgtaaacggctgaagtaaaaatttttaaagttaaccagaacggttaagaggaactttcttaggaaggatgattaatcatattacgaggatataaaaatgccccagcgaagccggggcgtacactagtggtaagatggtgcaacatcgtcaccttggaaccaggctgaattggaagatactttattagcatattcccatttttctgttaagtcatttagcccagcttgcatttttgcgtatgacgcttgctgatatcggtccattgttattaaagattttgtcattgtggacaaaagttttcgcctaccttcattgccataattgcgaataagcgctctattatattccggcactaaaaatgaaagcgagtgcccaatgtcagacaaatgctcagcctttaattttaatcttttaacggcaatagtttttcttaacttttcaatcattgtcaattatcctcggtttgctaatgcgtattcattagcattaatataatcctcagcaaaaacgatatcaccgatttttagttgaattacaaatagagagtctaaaaactcaggtggtttaatctcgatagacatttcttctttaataatttctattaccgcgcggtgtaattcatctttagtcatcatttacctgcaattaaaacaccgaccataaaaataatccagaacgggtaaaagaaaaccattgaccattctgcaactatttttgtcagtttctgtttagtagtacgacagctcgacgctggcattccaatacaccaagcaaattttctaacaaataaaaaaccgataattaaataagcaactacacctaatacaattaaaatttccataattacaccgccattataaacccaatgcatcccgttctgattcggttaattttgctaaagcttgttcgcgtactcgtttaatgcgctcttctttgtctttggcagtttctcgttgaatatcaaaaccatttttaacgataaatgaaagcgtctttccatctgcaaatataactgttaaagcttcttttgacgtgtctgaaacatgtccgtcactgccgtaccaggatttaccggcgacactctcacgcgctttttcatacgagtcatagtatccataatgttgagttctgcggtcatctggggatatcgtagcttcccagaccttttcaattttactgctaataatagttgtattattaatcatttttaattcctcatatatggctttcacaatttacccttaaagaattctgcccgggaagcacgtttaggttcgcagttccaatatttatctttacacggagtgctacaaaacttatgttgccaactacgcttcgtaataacctttcggcaataaggacaatacatttttgtccctactttcactaaagtgttgttatcgtaaattggtttcattacattataacgagtatctaattcttcctcttcaacgtaatcccaatcttcatctaatccgtatcccattatcctttgctccaattattaattatttcaacattgttacttctgagatttcacgccaatttttctcgtctgttgctacgaaatccgccaaatacaaggcagtacgtaaagatacattgcgaagacgatttacgttatttttcataaattcaagaacatctacaacttgtgaattacgtaatccgcgattttgcaacatccgggtattcataataacttcttcaacacggaccattatttcttcatttgaatgaacgcctaaatctaaataaactgaacgtgataccaaagcggcgagatgtggtgctaatttgcttccgcgttctaattctttatcaatatcaacgtttgtgataaaaactactgttccctcatattcaaattcatttgggatgtttttttcttccaagtaagaagaagcagtactccagcaaaccttgcgtttatcgcccgaatccaaagcagctttaagaagatttagaatatccatatcagaaaatacatccacatcatcaataagcagaactgaattagcattgcgagattcccaaaggcggcagtaaagaccaatcccggaaattttaccattgactgatttatattcaattctaccatcattgtgagcattttctaatgctttatccaaggtgtaagttttaccaatacccgcagctcccgagatgataagtgaacgaatgttaccatttataataccattcgtcataaatcccataacgttaaagcgcttattaatgcgagctttcatatcatctacgctttcgataagttcaatctttttagtagttccgctgtagctgatatctgatttgtaaacccaaacaccttttttcttaccgtcaatcattacgaatactttaccatcaccctgggcggcatcttcgggtttcaaagtttccgggaaccattcccctactaattcgaacgtcccggaaatttcttttccgaagttaatacctttattgatagtgatggttgtcattttattctccaagttaattttgttttaccatttatttggtatgaagtaatagtatcatcatttcataccgttgtaaacattattttttgaaatttgcgtttttaactgtaacttcatcttctgggttgaaaacgaactgcttaaataatgtttctgcttttggattgaaaatcatagtgaagaaagttaaagtaccaggttcaacttcacgaaccccacaaatatcgtattcaacgttatcaacaatgagtttgtttgagttcagtgcatctttagctttgattacagaagtttcgattttcagggtaactttcattttattctccaagttagtttttgttttaccatttatttggtatgaggtaatagtatcacagtttttggagcatgtaaaccttttttgaaatttttcttcaaaaacaacaaaacctcccgaaggaggttatttggagtatttttctagctgacgctcagcgaaccgtttagggagcatcaacaaccattttaagatatgccagagacccgttaagataaaaacaggtaaccataatagtaatacaaacaggtaagaagaagtgtcctcaatcgtccctcgtttaacaagggcatttgttataaaccagccgcatgttagataaactaacgtaacaatgataggcataatccaccaaagctgagctaaaagtaaagtaaccataattcaccggtagtaatggttgtgattttcaagataaacctcttccaagaacttgtcccagaatgtcatatcaattttgttaggcattccatttttagacgcagtcattgccagtgcttcaacttcatctacgatgtcttcaagactagcttggacatctttgaatgggattaatccttgcttaatttctaagatgaatggcgcagtacaaagaggatattttaagtcgcctgttttgtaaatttcctttaactgatatccagcacggtacgcatgactcagagctttccaatcaatgccttcgttagcttctgcctttcgtgctcgttcgccatattcactatcaagtttgtttaaagactgtttgagctcagtaacagagagtgtagtctggtatttgcgtcctaagacggtatagaacgtctgtgggccagttttctcgtggttatggaatacccattcacagaattcattctctggaagacgatgtttaatatcttcaaccttagtgcgtcggacccggactatatcatcttctaggtaatcaacccatttttcaggaacatcattaataatttctaaaacccgacgaagagcagccaggcgcgaaccttttacaccatatttggcagcttgtttcctgacatatccaagataagatttcatattagtggtgtaaaaccgccaacggttgtcttggataaatttccaaacatcaggaaggtcggatttgacaacaagtcccggaggagtatgaagcatatccaacgcgacagtttcacccgttgcggccaaatgaaagaagtatttcaatgaatacaattcatggtccacgtcgtctttggtgtttttagtttaagtgttattggtgttaaaatttgtgtggttcatagcccgtccaagcagaatgtctcgggcatttggaacaaaaatttctttaaaatcgacatcactctcaggcgtagatgtaccgtataaatgacttccaaaatagctttttacaactgttttcatattagccccaaaataatccgtacgcagaaagcgcgtcttcatctttaaaattaatcatttcaattttacgagtattttcgttttcaacaactacagcagagcagctatcattaacataaactactttccaatatccagaacttgaaggtttagaaagcatatcaccaacagccacaggaacattaatgaattttttcttatttacaactttacataatgtatatttttgagggtcaaaaacaagttcttcgcctttatattcaacaatagaaaaatgttcaactttgaaaaaatctgagccgttataatctaaaattccgtctttaaagtccacacgaacgatagcatccgggtctccaactactgtgccaaatatataattctggcgccctgcgtccggaaattctccaaaacaagttttacctactacacgtatttcgtattttttggttccagcgaggtcaatcaaaatattaagaatatcgccagtttcaattggcactagttccggttcaattactgtcttggattttttaaatgaaattttgctcatgtttttctcatagataaatatgtagacctttagcaaactcaataagagctttgccgttttcacaagctgtattagcttgctcttcactttcattatatttcgagaccagtgaaagcataccaattttttgatgaactgaataatgagtcacatcaatcatacgatattctcgacggtctggatgagattcgcgagatacattcttaataacacttgggttatgcattgggtataagcaccaatcatttaatccgcgaatcaaagccatgctagtaacaacttttttataagggaagttagtgatataacccaagaccctggtttcagggtccttcattgcgaagctgatatctgaataagatgctaacttcatttgtttaacgatagacaaagataatttcttctttgttgttacaatttcgatttccatcatttttcctgcataaattcaatatgtggacagaatttttctttgcgagctttaataaaagcgaagataatattaggctgttgttcagcataagcaccatttctggcagcttcccattttttatattctttcctttcttttcgatgttcgcggaaactgtaaattccattaccaatgcatataagtataccaaaaacaactgctagtataagccaaccaataattaacccaggaataaacgcctctactgaagaaagaattacaccgcattgggcaaacaaccaaatagctaaatctgcaccaataccccagctggcgaaacaaactacgaatatagcaaataacaaaaggaaagcatgccaaacacctttccaaaaataaggacataaagaatatggagcgatttcagaaccattcatagtaacaagtttatagtgccaagagcctttattgatttgcattattcatttcctttcatagaaggcagtacaacgttcaaatagtcacgaagtttttcagcttcagctttcaacagaaaaatttctacatcttcgtcttcataatctttatgaattatgctaacaatctcatagttttgtccttgacacttatgctcaataacatttaagttgagaacacttatttcgccaaaagacgcatcaattacattagcttcgcgaccaacacctttaaaattcatcacagtcattactatttcctattattaacaatacaattataattaaaagaactatagatattatagtctcaaacccataaagcgtaataaattctatcatttatgcttatcctgttcccttttgattggttttgttagcaatataagacattttagcagcataaacataagaagaacttttatgaataatttgagctaaatggggtagtgcttcagtatcttttataaacctttctagctgtgaacgacatattccatcttccaacatttcatcgtatatggtatacatcaaaaacccggtctggcccttgtactggcaaatttcaattcgttcattgccgtcataacaacgagtaacaagcaaatcacctacttcaagttttggaaaactcatcttacactcccagatattaaagttcattgtaaacttactttagcaaattcagcgatattattcattgtccactgtttcccaagcaagagttactacaatgttttcaatagattgcactaagtgttgttttgcaccagttgataaatcatactgacataacgaattagctaattcaattaactttcttttagtctcttcatgagctgtttcgtcatatttcattgtaaacttcccttaaccaattcaacgatgccattcacaagattgtaagtaaacaatggatgattgtattgaacgaacagataaacagcaataataacgtttttcatatatcctccatatgatgagataattatactatatctcatcatgaatgtacaatcaaaaattaaatgatttcaaaaacgctttttcatctatcgggctattatttttagcgtattcagcatcttttgggtcactaaattcattatctttcaattctgcgcagaaatgaattgctccaccaaaattagtaaaaactcggttcaaattataaaccgtgcttcgctctgaagtctcaacgcccatatcagccaaagaagattcagttacgtattctttaataccatcaaaacttttatactttcgaatatgtttaacaaaccaagatttagaatacccaaaatctaccaagaatggatgactcaaaacaataagtttgtcaatagactcttctgagacttcagtttcggtacgatgaattccatagacatgatacagaactcggccagggacaaaatcttcgaatttagctttcatgatattttcctcatttgttttgataagttaatagtatcacagtttttagaggatgtaaacggttgatatgaaaatgccctggactgatgccagggcgataggattactccaaggtaaggaggtacttcagttggtaaaatgctccaacaatctcatccaccgtgttctgtatagcgcgaggaattttatcataaattgcgtcagattcagttaagatgagttcaatcatttttatagtgtcgcttggcaaagtcttttgttctggaatgtccggaacgtaaggcttgccagaaaagcctagccattgttcaccaaacttatcaatcaagtcaggtaattcatcaaatataaaattgtatgctttatgacgagcatagcttttagtttcgaaatgagctgaatgaaaatatgaacgagaaaccattaacaagccaagatatctattggcttcgtttgatttgccgcctttagcaaaatcttcaaatttcatgttcttccttttcagtaaatcgattagtgtaatattctttagcaacctctacaccagcagcttcgcacgtgctagtacatggttgtctatcccattttgacttaacaaatgagaagaattgctcactttcccgtttattataaattttatattgagtatacactttaagcgcaccctcaaaatactcatcaaatttaggattaaacgtgtctgcatgtactggaacacacataattccaataagcgcagatagaaatagagctcttaaggccataatcggcctccttatttttgtccagtgttttgtttgtgctgtaatacagccttaacttcactgaggtatttttccagcttacgtttacgtgtataatctgctttcgaaccgtatagacgtaattcttcttgaaggtgcgaaataccatgcccaatctcacgacgaagttcgtccaattgttctacagtaagattacgaagttgcttttcgtttaaatgctgcatatacacctcattagttagtaatagtatttataaatgtagtactattattacataccagttaattcacaccagtgtttgcggcaaagagatacatacttatcttcgccacctaactctattgtattaccttctttcacagcgtttccattttcgtctatacgagccaccatggtagcttttcttccgcaatgacaaacgcctttcatttctatcaatttgtcagcaaccgccaaaagagaagctgaaccagtaaataatttacctctaaaatctgtgcgcaatccatatgccataactggaacattatagatatccacgattttacataactcatgaacattagcttcagttaaaaattgcgcttcatcaacaaatacgcaatgaatatctcgttgagtttgagcccatttgaaaaattcaaatatatccatatcaggcgtgattacattagcttcttggcgaagaccaatgcgtgacacaacttcagttgccgaatctctatcatcaattcccggtttgagaattaatgtacccataccacgttctttatagttatgagcagctgtaagaagagaagctgatttcccggcattcatgcttgcatagttaaaatacaattgagccattataagtccttcactaaattttccagatattgttctagagcattttcagcttttgttaatttatctgataattgataatatgtttcagcgtcgccataatcagaattaatctcaaacgacatatctttttccaaatcaattatatcactaatcatctgaagtatatcatgcttttgttcctttgtcatgtcgttatctcatctaaatattttttaaacgctgctttatcgtcatctaaaattttacgataagtttcccaatcatgaccagtaattaattcagcttcataattaatttgagaattcaaaatctcttcgactaaataaagtatttctttattttgctcagtagtaatcatactggctctcctggttcaatccattctggagtatattgaaagaatccttcaccaacgtgattcttacacgttttgttggagcaatataaccaccaatcccacgcattataaggtggagtattcatcagcccgtcattacataaagaaaatactacgggccatttacaatctttacaacggtgtttttcttttatatgagtttgcattttagccatcccaatccacaacaataatatcaatgtcaggagtaaacatatcaataatcaactcaattttattccaatcaccgcctgcgataccggcaccaatgcgaggaatataaatcacaggcctgaataattgtcttttagcccaacaatttaagccctggaaagcatagagtaatgcaccataattgatgttaggacctggctcgtattgggtataaagattaaagcaaatttgaccacgttcgccagtcgcttgagtgtaatttcccaatttatctgggtctgcatgctcagtttcgtaaacatcaatttccaaaataggaggatatgccttagctagttgtccagctactcctgcacccattgtatggaaacaattacaaccgtgagctacattgtgaccttctaagaatatttttacaatatcgcctttgatatattttttaatcattttttcaccatataagttgaatattcgtcaacatcattatatactctaataaaaaatgtatcgcacgttttcattatagttttcgctgtgttataactatcaaatgacgctatcaaaaacgattgctctatttctcctttagcgcttttctcgcagcccctgatattaaaagaaccaacgggtataacgacttggtcacctttatcattataattataaaacgatataccaagaagctctttaattggaatataaattaattctacatttcgtttaaaagaaccggaagtttcaatttgtttaccggttaatgctaaaacctcttcgggatagcctgggagtttttcccaggcattcaacgcaaatgtaacgaataataaacatattaattttttcaattagttaatcctctcatgtattgctctaataatctatttcggtaatcaaaacagatttcagtatttggagctttatcacacagattattaagttgtttagttttacgatttattaaaacgttccactccgattttgatttagatgctcttactccgtcttcatacgccccgtccaattgcaaggacaagatatcaatacattctgcgtttgtagaacaaaatgatttagcgattttaagcgattcctccatattatcaccagaagcatttgctcctgatataagcaaaagactaagcacaagagcgattttttgcattattctacctcaattctcaatagtttatttgactttagataatatgctttttctaggacttgactagcatatctgtttccagatttccagttgttcccggcgttgtaacttgcaatagcttttcgcatattaccattatatctatttaaccaataagaaagttcaatatatgaccatgacgctgaattcgagcgtttattcaacattttaattatttcagcatcagtcatttcccatccaatttgtttcaatctagcccgcattgttggaagatagttttgaaacattccatatgcttggtgaccagattttcctttggttttcaacccagcggaagattcttgccagaccaaagctgccataatataacctagccctttattatcatagcggtttgcttgtgttttatatttgccatcctttgcaaattgctctccaaactgataagcataattcagattttcgagttggacattactgaagttgtgctcggacccaaaggccattgaacaaaaggccaatagacctacgcatagagcttttttcatgtttacctcatttaaaacttaaagataaattacgtgtttgctagtttttccttcgtatctatttgtattaacaaaagccatccgacactgcatagaataaggagtagagaaagttttaccaggagtaggttttgaaacagtgaaaccaagccaaagagttttgtcagtgatttcaaggcgaagaggtcgatactcaatatttgggtctaaattttcttttgcatctggtcgttccgggagctcaagaaattcattaacttcgacaacatgatttgaaagcttgtgaaaaagttcaaaaacatagttctcgtcaatttctcgttgaatggcgcggtccaataaatgctgagaatattttacatgaaaacctggaactccagcggattcgcatgctgcttttatttcggaattaattttagcaaattccgtttcaaaaagacgacgaagtttgttgcggcggatgaatacttctgcgttagttgacatttatttctcctctttatgatataagattataataacacatccagaggagaaagtaaacggtttgtaggcttaatgtcttagttgtatttcacttcctcatttgtagcaatatcatattcaactaaacgaataggctcttccatcccatacccactcatagagatataagtggatggataaattgtttcttttccgtcccaagtaccgccggaccatgcttccataaagtcttgttcgccggaattaactagccaatcaacaaatgatttaagggccatttccgacccttcaataacgattttagccatgatgcttacaccttataatgataaatcttattgcattcctatcacaaaaaattgcggtcgcgcagtttgattgtttttataacaatcaactcgacaacaatactgcatggtttccatatgaacatcgtaaagcttatccatatcaggagcttttacaggttcatcaattatatacaagattcttgaaagcgatagacctcggaatttgtatcctttattgcctataaaactacgcacagaatcagtgaacaagcgaaatcggatatcatcattagaataacgcgaaaattcctttttaatgtgatttgcggaaagttcagcgtatgccgatttattggataaaacaataaccgttccactatcatacagccaattggcggcgaagttagttacagcgattgatttaccagattggcgcccaccgtctagtcgaagagtgcaatattctttaagcagggtttctaatggcggaacccagccatttttgcaaatttcatctactctagcatcagaatgatgtgtaaaagcattcatcagggatagatatggtgcagttaaaaatgttctcatttgtttctcttaagtttgtggcccgttacgtcggcgcatgattggtccttctaccgctatgtaaacctattaccgcacttgggctcgaccttattacaggttggaaaaggttcatcactcagaggacacgacgttcatgaggagaggctgcgttaccctttattaggcaggaaatgtttaagctttaccgcctgaaacaattccaaaattatcaaaatcgtcttcaacatcagaagggtctacctttttaccaggtacaacataagcaatttcgcggcgtttagcatgagtaacaatttctttacctaagccgttaaagaaagaatagccatttcgttttagagacccaaagtcttctgcgtcgccagtaaatgcacgaactttaaatgcgccttttggagcttttactgactcgttaataaattcttcgtaagttttcatttaatttccagtattaaccaaatagttagtaacgttctgtagaataattataacatcatctacagaacgtgtaaacatttaaattatttgattgatttgacgtatcgggtaacacctttgtcattgacaaatgcaatcttaccgcggctgttatacattgtcacagtacgctgatagccataagtctgtttacccgcctgataaccaatcaaagcgaagttatcttcaccaggcttgttcagtttagctttgatacgttcatggccataagccattaaacttctctgatcacctgtgttcaatgctttgcgaacactttcatcgtcaacagaggaaacaaaatcctcaaatgaactgaacttggcgtggctcttgttgtaaagtttgccttcaactacataattatagcccataacatccatatcaacttcagtaccatcttcaagtactacagtgattttctgacctttcttgtatttcagcagaccatcagtagattcgttaatgataatctgagaaattacgcgggcttcattgataaaatcttcataagttttcatatgctttccaagttcctgtttcaaaagttttaataacgcggcgggcgcgattagttgtttgtctgtaccatttagattgagctaaatttacagcagctctatcccaatctttagcttttagtaaagccatagaatttttaaaacccgctacaccagctacacccatttgaaaaaccatgttaatcagagcagccagacgaggttcatcaccaataaaagaattatatacggggcgtaatacagaattagaaagaattcctgaaatagctttttcaacgtctgtattaaaaattttttcagcttccgctttagtaattttaccggatgtagcccggccaacaagacggtccaactcgctaatagctaccgatttattagggtttttagtcaaaagatgacctataccgacggtccaaaaaccttcagtatctttataaatttcaagttttaagccttcgtcgatgcgaagcatgtcaaaaatgttcataatacctcctttaatagaggtatttattattcccgactttttacaaaaaggtctgccatgaattcttccaggcgagtttctacggacgatgcaacaccggatgatgtagaaacaagataaccgctcataggactgaaactttctaaattgctttccatttcgtatttgatttgttcttcggactcgttatcgctaaatgaatagagataattatatccctttgaatttacgtaaattgctacactcatcatatcacccatgaatagcgcatcattaacacaccagaagctgctaatggctcaacagaataaatccacgtttttgtatattttcgacataagtctactaactcacctatgtcatgaaccacaacggatgttaaaggcagagttatatgacccgtattagttaatactatcgctttcgggtcatcaataactaagtgctcaatcttctcgtcttctaaataatttacaagttcttcgagtttattcgtataccatccaaattatatagtacgtccttcgacgattttagcgaaagaccaatttttagtaatgaattcaattattaaattcaattctttagcttcgacaatatcagttacgtggttttcgtgatttccagtattcagaacagcacgaaaacgatcgaatttaccaaaatcattagttactttatcgatagttacagtgttaaatttagccatgattttattctccaagttaattttgttttaccatttatttggtatgaagtaataataacacatccgtccatggatgtaaacggttgaagtaaaaatttttaaagttaaccagaacggttgagagattggatacagaaaggataatagattataaatctatacaaataaaaatgccctagagttaaactagggcgtgagagattaaatcttatagaacctgaacggatttatatggaaagcaagcattgctctaggtatcggacgggataacaagcttttaagtttccacccgcaataaactcgtaggtaaaattgaatttttccaattcttagataaggaacagcagcaaaaactccccaagcgtcatcgttccacataaacagccatccatgatgagctttatccggagaacccgattctacattagggtttccacgataatgaaactctgaagtagcggggcggccaaggacataatatgcaaagttataagctttatttcgccacagccatgctacgcgttgaaagtatttacctaaaactggaattttcgaccagcgctcaatatgtccacggtctccatcaataggattgtcgtatgtttccatccaacggaatcctttaggtaagtgaccagtcttttcagaatagaatggaacaataaatggagccaatacaacagctaaaaccatagaaatgaaatctagtggaacaagaaaaatccatgaagcgtattttagaaatttagtcataatctactctaattaaaatatttattgaagcgaatttcgaattctagtcaattgagcgcaaaattcccgctccgtaagaatagtttccgtttctttatttttacccattgaatgagttaatgtagcgttacgggttgctaaaagagctcttttgaacccgtcgttttgggacagtgcatcatatgctcttgtgattaaattttgataagcctctgacgaccgatgaataggaacaccacgccagtacaatgtctgagttggccaccatcgtttctttttacctttgaatttagcttgtcttccaactaaagtgcaaacatgctcttgcatttcaaccgaagaaaatttgagggattgtaaaaatccctccattgaagcacatttaactccgtcaatttcaaatgaatgtggagcaaagttgcttaatgaagacgacggataacccgaccctgaaccaatatccatttaatcaaccttataaacaaatatggagggatttatccctccatcatattaatactgactacgcattttagctttaatggctttaagttgcggagaatcattgccgtatgctttccaaacgaccatttcagcaccgttgttttccatggagtgaacgataattttaccgttcttacctttttctacaccagcttgatagcgttggaaatcgctgttagtaattttcttaaagtctttgatatcaccttcaatctcatatgagaaattgaagccgctccacttactttcattgataaattcttggtaagttttcattgttatctgcctagttaataaaaatatatttattatagcataaagagttgaaataaggatttgaaccttatatagttgcacagacgtatctgccttcgaattcctcactaagtttaagcaactattcaattaaattggtcgagacagaaggattctaaccttcaacctacggtttagaagaccgttgctctatactattaagctatgcctcggaaatagttttgcgtctatatacttctccgtcgtaatatttatcgacaagacgctttacttgactgtgtgatatttttaatattttagagacatcagaaacccacccaaatttagttttatccacgtcttttattaagtttaattgagacgtagataattctagacctctaatcttggctttagcactaccagcttttcctagtttaacaccggccaaataaagttgttccttggttcgagtatcaactctttttccatttgtatggaggtctttattattttggctattagtagcgcaataaacattttcaatagaataaggtccagaatcaccgatgcgacacatacaatattgttgagctttattaccgcgtttttctaattttccagttgaaatccaccaattaagccaggactcatatgtaaataaccattctatgtttctagccttcgcctttcttctttgattttcatatttaactttcatgtttattgctttcactcggctatccaattttaagaagagtgagagtcttagtcttgtacggagtaaccttttatacattggcagctatcgcgcgattcgcacttccgataaatggagttaccgttacactctaatttggctgggaatactgggctcgaaccagtgcatcccggaatcaaaatccggtgccttaccaacttggctaattcccaattatttggtggcccttgctggtttcgatccagctacctatcagttatgagctgacagctctcccattgagctaaagggccgtaattctattatgaaaggctctatgttaagaacctttggtaatagagggtacttattaataataacactaatttcttaaagcaaattaaaacgtatgcagatatattttacgaatatgtttcaattttaacggattttcaaaaatattgtagtcctttcggccatcttcagttagtaaaaagaactcaaaattgacagtgttaaactcacgctcatttcctaccattttaacgacagaagagctccggtcaatgtcaatatattcaacttcaaaatatgataaaattccgtgttcttcaattaatgatttggcagccacctggtcagttgcgtatccgttttcagtattatttacagctgtgtagagaatcatcaccacactcctttgtcaataatagtgtgagtgttggcattcacaatttgccaccatttggcatggtagaaactgtgtttaggctttttctgattttcttcgataagttcacgaagctcatcttcagaaaaagcagtagcaattaaatcattgtatccgcccgcgggataataattatcaccgccaaataacaagaaattgactttagtcatatttaccttttaaacatctgatgagtcatgtagacaataaccgctaaccataaaaatacactattaatcgacgttaagacaaggcctgctataattccagcaaaagcaaaatcccactcagtgaattctttcttgtcaaaattatagttttttctaaaatatttctgaatttctttaagctttttcattagaaattttgtccataacaaatgatactacgccgagaaccgctacggcaatgaaagcactcatagtttcagctttagataaaattgctaaagaccctgccatgatagaaaaaccaaagccaccaagacataatactgcagatatattacgaataagttcacaacgtttcattttattttcctcatttgttttgatagggttatagtatcataaccctcaaggaaagtaaacggttaaatcgcgccgtcgtcttttgcaatattaagaatagtatcatacaattcctcagcttccttccggcgggccgagattttagtgtactcttcatattgctcaatcttagaaagcatagattttacagatgaataatggtttttcaactttttgatttcagaaatagtaaaacgtttatctaggccttcgttcaaataagtcgattcgttaacaaattctttaaaagatttcataatagttccatgtttgttgatagagttatagtatcataacctgcaagaaatgtaaaatgtttacattttacttccacgaatgacaccataggttgcgaaatcatcagtgatatctttagcagaatttctacctgtttcaatatatgcaatatcagattttttcgaaaaagaagtgattttgttataattagagtctaaaaaggtgtaaccattacgcattagattttcaaaatcttcagcatcgccacgtcctttaaaatctacccatttgtaagtgtttttcttactaagacccataggccatgatttttcggtaagaaaagcttcgttaataaattcttgataagttttcattttaaatcttctgtccaattaattttaagtggttctttatactccctgtcaagtataacagggacttggactatgcccgaaaattgggcaggtcctacgttgtagctcagtgtgatgtgtggagaataatcatcaaaatcatgagtagctccgagggcgcgggcgtaattgtgacgaaatttaagatagtcagagtccagaactaaaaccaatgttctgccatcttgcgtgtcccagacttggagttctccagaatttgctacttcaaaacttccagtagaaaccacataaggcacattaacccgagaatagcaaatagtgctatgtattttatgtcttggaactggattaggaaccttcaattctcgctgaaggttctcaatttcatctaatgtgctatcgctaaattttgctgcaacgtagagtccagaactaaagtcctggaatttcatcattcatcatccggcgttgcggttaactcatcaattttttcaagaatagcttcaaaggtcacttggccttcaacaacttctagaccgattttttctacaattttacttaccaaatcgcttagcgactgaaccgtgttagtagtttcagcaaattgctcattcaaatcaaaaagacgagctttaagaacagttacagtagcttcaagctgtttaattttatcagtcattttaaaacctttttaatttttgcataaagttcttctaaagaaccgtcattagtaataactgtatcgccttgtttaataggcaagccagcttctgttatatgagtgtcaacttttgaattgactgagctaggacgaactacatgaattaccgtagcacccatcgccctagccgcgtccaattcatggtcttgacgagtgtctggtactataaaataatcgagattttttccaaaattatctaaatagaccagagaaaaccatttaacccaatacatacggtcgaagtgattgaccatgatgtctgtcccaagggtctgcatgagacgacgaactgaccattcatcttcaatactatttatagtgtctttaattattgaaagagaacgatggtcgatatatccgttataaaaataagcatttttaatagataacgtaccacttagccatgacatggcagactcaatgatgtttataacttgcacctttgaaattttcaaatcaacttcacggtcataatcaattccttcccaatcacttcgatttaaaactggaaacaatttaccgaggtcttttgacataatagcaacttgataagcatagcaaagagcatctttaattggtccagctaattggtgcttaattgcgccataattttccattatgtaatcagcggttgtgtcctttccacttcgttttttaccaatcaaaaaaattaatttcattatttcctcatagatggatgcattccaatgcgttgattagcagcctcggccatattttgccgtgttgtaataattctaccatctttttctatttgcaaatacttatacggcatgatggcttgacatgttacagctgggtcagtatcttctgtgtagttgtattccacttcgcctaagtcgcttatccaacatccatagtaatgaatagacaaaacaattctagttttgctattgtctaaaatatgaacactgacgtgttcaggcatactgccgtcgcgccatgcgaagttatctccagtttgatagttgtttatgcctatcatccattgatacatctgcagccaagagtccagttcttcatccaccaaaaatctgataaccagtgggtcgaattcgaacgttgaaccaggaaggttcgctcgtccaagacccatagtgccagagggaatatcagtaacaggaattcttataccaggtatattagctgattgcgcgttaagcgtaaaaccttttgtcaagccaacatccggaatgtctacaacaaagttagttatgttggtttgattgtatatctgttgtgtgttcaatctgcttttctccagatgttgttataatgaatttgtccttccggttaatcaaataatcaaccagaagaacaagaaagtaaatagaattatgatttccatacagcgtttgcgctgaaacgtttacctttactttggaaattctgaagaggtaacataactacatttgcccaatcagcaggtttaatttcaatcaaactcccctttatatgaccaggtaaatatgctttaatcattttatctgcaccttgaaatcctttcacttgactccagttcactttcaatttcgttttgtttgttatagttggagtgttagaatactgctttagcagctcttcaagaaattgttgccgagcctttggcggaatatagtgtaagttcaatccatacattaatgtagaattgccttgtttacccattcctaaataaattattaatggaaatctatcccaatatggaagagtatctttatgcttagcatcatacatataagcatataattttccaggttgaggttttgttacttgatggcctctgatgttctttttgatagtctcggagaaccatttagcagatttattattgactgccgctccttcatttctaattttatcgcgaagcgaagacctgaaactattaatcattatcagttgtctttcttgctttgacaacttattagcaggttttccttcaagtttagcaatcttttctgcgaactttatcttagaagaataccttgacatagccttagtgaaagtagcataaggtatttctttggactcagcaaattcttttgagctcatccctttggatttagctttattaaattcaagccctatttcaacccatttcttttcatttcttgaaactggtttaggacgagtagtagaacttgcagcctcgccaatgatttcaaaaatagccattatcctttccaccctagtcttcgaagaccatcttctgttaataatctgaactgtatattatttttttctgctacttgtaacgcagctttccacttatcagtatttactgaatatgtataaattgaatcgatgtatcttttctttgctgctgtagttaatttagcaggtttaggaggagggactgtttcttttttgggttttacttcaaagaaaaactcttggcctgtatcaaatttcacccaaaaatccatgaaatatcggcgttttttgccgtcggcattgctaaagtatggaattaccacttcttctgaattccacttcactacttgaggatttgtatcgcaccatctcataaaaaactgttcccatgaagagcgataagttattttacggatgtctcctctatatttttgatggttcgttggagtgaaccttcctgaatatgccattattatctcctctaataaatactaatattatttatatgtgggagaggttccatgattttttcttttttcgacccaattgattataaggcaaaaactgtcgataaaaattccaaaactattaaaatgactgacatctttcgcaattacaaatcttactttaaacgtgttgcggcgggttatcgtctaagaacttattacattcaaggaagtccaagacccgaagaactagcttatcaaatttacggaaactctcagttatattgggttcttttattttgtaatgataattatgacccgtattacggctggataatttcacaagatgcttcttatcttgcagcaatgcagcgttatgaaaaagcgggaggagaacaagtactttatcacgtagatgaaaatggcgagcgctattataatctaatagaagaccctaataaccctggtacttggtatgataaaggcgacctagaaatgaaatatcctcaatacaatggagctctcgctgcagttgatatctatgaagctgctatcattgataatgaaaaaagacgagaaataaaaattatttctcctaatgatatcgattcgtttattaacgacttaattagagagatggagattgcttaatggatatgattaataacaacgtcgaatggtttgtcggcgttgtagaagatagaatggacccgttaaaacaaggacgagttcgtgtgagagttataggtcttcatccattccagaaagttcaaggccctgtatcgggaatgccaacagaagaccttccatggatgagtgttttacaacctataacatccgcatctatgtcaggaataggcggttctgtcactggacccgtcgaaggaactcatgtttatggtcattggttggacaaatatcgtactaatggtttagtgatgggcacatacggagtaaatgccagagttagaccaaacactacagagggtttctctgatccaactggccaatatccgcgttacttaggtaatgatactaaccttttgaaccaaggcggcgaagcgggatttgagtcaatccctaacgttattcaagataataaccttgatacagctattaatcccgacgacacagatttatcaaatattccagaagataataatccaaattatactatagaagctatgcttcgccgcgacgaaggtcttcgtttgaaagtatactgggacactgaaggatacccgactatcggtattgggcatcttatcatgagacaaagagtccgtgatatgactcaaattaataaagtgctttcaaatcaagttggaagagaagttaaaggaaatcccggttctatatcaatggatgaagcatccgcattatttgctaaagacctagctgatatgcaaagagatataaaaaccaactcaaaagtcggtccagtttatgtcaaaatgaataaatcgagacaaatggctcttgaaaatatgagttttcaaatgggagttggtggtgtagctaatttcactaatatgcttaaagccatggatgaaggccgttggaaagatgcatataacgaatctcgaaactctttatggtttcagcagactaaaggacgagcttctagagtatctatgattattcttactggtaatatggaatcgtacggtgttcctgttaatccggttcaaggaaaatctgttggtgctcaattagctactgttaaagcttcagttaatccaagcgaccctcctataccaagtgacagcagaattctttttaaagagcctgtatcatcatataaaggacaatacccttatgtccatacaatggaaactgaatctggtcacattcaagaatttgatgatactccgggttatgaaagatatcgtcttgttcacccaacagggacatatgaagaagtagctcctgatggaagaagaactagaaaaacagtaaatgatttatacgatattgctaatggtgatggaaattatttagtttccggtgataaaaaggtaaacgttggtgcagatgaaacatactataacatggccaacagattacatcaaattgacggaaataatattttgtttatccgtggagatgaaacaaaaactgtcgaaggagatggaacactttacgttaaaggaaatataaagatagtcgttgacggaaatgctgatatcttggttaaaggagatgctaaaactcaggttgaaggaaatcacgattatacagtaaacggaaacgtaaaatggactgtaaatggtagtgttaatatgacagttgctgggaattggtcagaaactatgagctcaatgtcttctatagctcagggccaatatactattgatggcagtcgtgtggacattggtttataaatattaattattgactaactggaaattatatggcccagatacttcccgttaatacatccctcggcgatattcaagaaggcggggctgttgatttgactttcactgctcagcttgatgctactgatacattaaagtcaataaatataatagattatcagccaacttctggtataatagttgaaggttccagatactacggaaattataatagcgtattttcatttggagctgatgcattaaaatatcgtgaaggcgatgaattaaaaaccgcagcaacttgggaggatttacctcctcccaaaactgcagatttatatatgtggagagcccctagttcactccagagaacttttacgtatacggttgaatgtatatatgaatatcagtcggaagagtcttctggcggttctggtgaatcaggaggtacaactcctccaccggtcgaacgaagaataacaaaaacatatactcaattagtttacggaaattggagtcgttggggaaataaacttcgtgaatatgtgtatgcggggaactaatggcaggtttaagttttaatcaatgtataacatctggtcatgatgcatttcctcctaccgtaattaattcaactcagccaaaagtgtttactggcggaattccggtattagttgatggagaccaaatcactcctcatacacgcatcgtaaaaccatatgacacgcacggtggagcagtccagccaagaacatctaaagtttatgtgactggaaagaaagctgttcaaatggctgaccctatttcatgcggcgatacagtcgcgcaatcttcttataaggttttcataaaataatggcagacccaattaactatcaattaacgcgcaccgtaaacgctattccggacatttttgtaggaggaacattcggtgaaatcaagagagatttacttgattggcttcgcgggcaaaacgagtttctagattatgattttgaaggttctcgtttaaacgtattattagaccttttagcttataatacactttacattcagcaatttggtaatagcgcagtctacgaatcatttatgagaactgcaaacttgagaagttccgtagttcaagctgctcaggacaatggatatttaccttcttcaaagtcagcggcgcaaattgaaataatgcttgaatgtactcatgctttaaattggaacactataagtattcctcgtggtacaagatttttggcgtacgcgttagaaacttctgttaacccatattcatttgtaacaacagaagaagtcacagcagttcgtgatgctaataataagtattttccacgaattaaattagttcaaggccgaattgttcgtactgaattacgatatgataaaatgaagcctatcattattcgtgattccaatattgaccgtaatgaagttaaattatcggttaatggagttgaatggactaactggacccgccgctctatggttcacgcaggttctacatctacaatttattatatgcgtgaaactgtagacgggcatactgaaattttcttcggagaaggtgaacagagtgtgtcagttgcgggtggggctcttgaagccaattatattggcggattaaaaccggtacaagactctaccatcgttatagaatatattcgcactgaaggagctgctgcaaatggggcgacagaattcagttatgctgatactttaaattatataacagttcaaagaatatatgaaaaccctgatgactccaaagattatgttggagctgacggcggcggcgaccatgaagatatagaacgtatccgtgaacttggcacgataaagagagaaactcaacaacgttgcgtaactgcaaccgactatgacacctttgtttcagagcgttttggctctatcgttcaagcagtccaaacatttactgatatgtataaacctggttatgcatttattgctatcaagcctaaatccggtttgtacttaacttcagtacaacgagaagatattcagaactatttgaagcaatataacttagcacctattactccatctgttatatctccgaactatttgtttttgagacataatattaaagtatcttatgcgctgaataaattacaagaaacggaccaatggctcagaggcaaagtcttagaccagattgatagatattatattgacgaagttgaaatttttaatgcgtcatttgctaaatctaagatgttaacctatattgacgatgcagaccatagtattattggttcgtctgcgacaattcaaatggttcgcgaagttcaaaacttctttaaaacaccggaatctggtattaaatattataatcaaatgattaacaggtcattagagtcaaactctttcacttttcacggtgaagattctgattatatggttagaatagttggttcagaccccgaccaaaatggaattggtaaagttataattggcccgtttaaagacggcgatgttaaagaaaaccaagatatccagccttacatgggaaccgattttaataagcttaatgcagaaaatcgtgataagtactatgtaattggggaaattaattacccaggagatataacttattggaatatttcggcaatagacttaacgtctgaacaatttgaggttcaaactatcgagctttattctacaccaaaggaagataacattttcacaagagatggttcattaatagtttttgagaatgatttgcgtcctcaatacacaaccattgaattggagcctatagcacaatgacagtaaaagcaccatcagtaacgagtctcagaattgttaaattgtcagcaaaccatgttcatattaaatgggatgatgttggtgctaacttttactattttgttgaactcgcaaacacccgcacatcagaaggggtctctttagacccctctagatatcaatggagaaatctaggatacacgtcagaaaacgaatttttcgaaagtggatttattcgaccgaacgattattatgttatgcgtgtgtcagttgcagctcaagggtttgaacgctctaattgggtacaaactgaagaatttcaaacatttttaacaaacgcatatacgtttcagtctatgctcgaaatgactttggctaataaattcatcagagaaaaatttactaataacaatcaaagttatgtagatttcaaccgtgatacagttatggcggctttaatggatgagtcatttcaatttagccctaattatgaaatagtatcgtcaatttctgaccatatattatttgaagaagaatatcatgaaatacaaagtagcatttctgcggtatgtaaagacataaaccgagtaatgctaatggaatctgaaggaattttatatctgtttgaaagatttcagcctgtagtaaaagtttctaacaacaaaggtcaaacttggcaagcagttaaattactaaacgaccgtgttggtaatccagtttcccgtactgtgtattatcaaagtgaatatacaacttatttgcttggatacgatagaattttttatggacgtaaaagtaatgatataagatggtcttcagatgaagttcgtttttcttctcaggacgttactttcgctaaaattggcgaccaacttaaacttggatttgatgttgaaatatttggaacatatgcatctttgccggcagacgtttccagaattgctgaagctataacaactactgatgatttcatatatgttgtagccagagataaagttcgttttgctaaaacgcgcaatgcgcctattgaccaagacacattatcacctacattcggtgaaaaattatttgagccagaagtactaacaataactggtaatccaaaagcagtgtgctataaaatggaagctgttaatggtaaagtttttgctcttattactggcgaagttaaagaagacaggatggacccgactgttccagaaaacgtaatagattctcaatcaaaaggaatttacattcttcagtccgataatacatttaagcgtgtatttggtaatacagaagaagaacggcgtcggatagagcatggttatacttctatgtctaccaacggaacagaagtattcatttcttctagcaattttaaatttttaaatgaccaaattgtgcaagacccagacacagcgttaaaacatgatttgttaggagccgttaaatatgaatttccgcgcgaatggctttctgataagcattatcatatgatgtcttttagagctgacgaaaaatctggttatgaacaatttgttcccggcgctatgaattattatgctgaaccattttttagctggtctagaaaatctggtactcgctgctggattaataattctgatagaattgtggttgtttattcaaatattactcatacaaaaataattgacactcatggttccggttctccagaacgagttttgcatgaattctgggataaaggcacttgtactgtaactgctccaaatgttgaattcaacggatttactaaatacgcttctggtattctcttttataaatcttccggtgaaataatttcatattacgaattcaattatcgtgttcgagatgaaactaaaataatttggaaaccgtctgaaatattccttaaagcttatttgcaaaatcaagagcgagaaacggaatggaagccagaagaacgagacggaatacaagacccagatttaagaccgttgattagaactatgatgcctgacagctatttgctagaggacacaaattttgaaaaattctgtgacgcatatattcaatacttatctgatggatatggaactcaatataacaatctattgaatttgataagaaaccaatatccacgagaaaaagactcatgggaatatctttggtctgaagtatacaaaagaaacatttatcttaacaaagataagcgggatgcggtatcaagatttttcgaagctagacgttcagatttttattcaacaaaaggtattgaagcttcttataagtttttgtttaaactcttatataacgaagacgttgaaatagaaatagagtctaactctggaaccgaatatgacatagttattggatctgattctattaatgaagacttagttggtcaaaccatttatactccaactggacgttgtaatgttacttatattgaaaggtcttattcaaaaggaaaacttcagtggaacgtaacaattcacaacttattaggacgcttaattcaaggccaagaagtaaaggctgaacgctatccaggctttaatggaatgatagttagaggtgttcgtggtaaggaattagtagaaaatactattgattacatcaaccgcaaccgttcttattatgttatgaaaatacgttcaaatcttccaacatcaagatatcgtaatgatgtgttaagatttgttcatcccgttggatttggctttattggtattacgttattgactatgttcataaacgtgggtttaactatgaaacatgtccaaactataatcaataaatacaaaaactacaaatgggatgctggtataccgactttttatccagatcgagtagcatcattagattcaagtggaaatgtagaaaaagacccagtcacaggtgaagtaatttatcttcctggaccaatgtctggtattgcatatcctgtcccggctgactatgactctgagaatgataattctattttccaaggtcaaactcctactgagcgtcggaaatcaatgagtccgttattcgatcaatctgccgtaactttttctcaattcagagacttagttgagcagcgtttgattgataaaacaggtctgcctcgcgaacctaataatccaacacaggttaaaatagatgaatgattcaagcgtaatttatcgtgcgattgttacttcaaagtttcgcactgaaaaaatgcagaatttttatgattcgataggagattcggcaaataaaaattcactttatataactttcggtcgttctactccttgggctgataatgagaatgaggtggggtttgccccaccttatccagttgacgatactcagggcgtagttgatatgtggactaacatgatgggttctgtaaaagttatgccatcaatgttagacgctattgttccaagaaaagattggggtgatataagatatccaaacccacgtaactttcaaattggagaaattgttgtagttaatagtgctccatataatgcaactgaggttggtgctggttggttagtttatcgtgtgcttgatattccagattctggaacttgctctatacaagatataaacaacaaagatgagtgtattaaattaggtggtaaatggacctcttctataccaagcgctaatcctcctcgtggaagaggtgatgcagaaaaagggactttgactatagacacaaacgatggatatttatgggagtatttgtatgaaattccaccagatgtgtcaattaacagatgcacaaatgaatatattgtcgttccttggcccgatgaaatagctgaagacccaatcagatggggattgcagaataatctttcttggcaacaagatgattatgggttaatttatagagtaaaagcttacactatgagatttaaagcgtactttgactcagtttattttcccgaggccgctcttcccggcaatgatggttttaggcagatttcagttatagtcaatccgcttgaaaagaaagataaacctaatgaccctaatgttaaggctgaagatttgtactacaagcctcatgatttggaacgtcattcaggcgagatgatttacatggaaaatagagctcctattattatggcaatggaccaaactgaagaaataaatatcatttttgaattctagcagggactccttcgggagtcccttttttattgcataaatataaatataaagaatagacggatatcacttgaggaatagatatgtataatcaagaaccaaaacaattaatcgacgtcggtgaaatcggtaacgcctctactggtgatatcttattcgacggcggtttaaaacttaattctaatatgaacgcaatttataattcgttcggtgaccaacgcaaaatgtctttagatagtggacaaggaactactggtcaagttatccatgccacaggttattaccaaaaatcagaaaatccggctgtagattttcaaactccggtgcaaaacggctctatgcatgacattgatacatcagctggtccagtcgttgttagattagaccaaggtgtccgtggagaaggtgtgttttttattaactcaagcggctctatttcagtccagaacccgttgacaatagaacctaaagacaattttgtcggcatctctggaccgcttgtagtgacttctccatttagtgaaattaaagtatggtgtatttctgatgatggcggacggtcagtatggaactattcaatcagaaacatgtttggggattatcatgacccggtccaaggaacttggcaaataccagtcgcgggggtagttgattttcctctttttcataaaaatgaatatatagcttcaaaacttttagttacttctaggtctaatgacggtaaaagaataaagtcttccgaaataaatatattaatagatgtagtaactaatacagtgatatctactgaatacgctgttatgcgagtaggagcgttaacagaagaggacgatatcgtaacttattcttttagtataagcggaacaggaatgataacgatgactgctaacggcgcatcaggattaagagtagctgttaaatctatcacaactcagaaaatcggggcagcacaatgaaacaagatttaaaaattggagctgtggttgatgacggctctggagactacctgcgtaaaggtggcgaaaaaattaataataacttcaatgagttgtattatcaactaggcgatggtgatttcccgcatgcagcaggcgcgtggaaaactttcaaatcagctgataactcaattctgacagccgactttgggcgttcttatgttttagatacaaccgctggcagaatgacatttgaattaccaaagggaaatactccagattataaccatgttattcgagttagagatgtatttggtacttggcagacaaatccggttaccattactccagcggcgggagatactctgaagggtaatccaaacccaatcgaatttaatgttccactgactgatttagaacttgtttattgccctcctggacgctgggaatatgtagaaaataaacaaattaacaaaatcagtaattcagatttagcttctgtaattcgtaaagaatttttagtagaaactcaagaccaagttgattttttagacgtttttgacgggtacgattataataaagctaatacccaggtctatcatcgtggtaacattctttattatggaaaagaatttagcgaaaatagtgattacggctctcctggagctacacccgatgaagtagttgctttaaatggtaaagatattcgtttacgtcaaaaatgcaatattggtgatacagtaatagttgtttcttacgttgatggtattagtcaatggcgtagttcttataataaacgccaaattatgttactggattcttctaaaacaaatcaagtttcagttcccggttcaacattcgtaggcgatttgaaaaatactgatacattcacagtagaaatgtttggattgactttacatgacccgataaatcctaattcgttggaagttgaatttaacggcataagtcaagtgttagtgggaacaaccggtttgcctgaagtctattgtcaaggagctgacgcagatacttatgagtcttgcgttgctcttggaggaacttggacttcatctaatatggattattcagtcaattttgatacagataaccgtattgtttctatcacaacagaccgagaaatggaacacggcgatattattaccataaaatggtataataacaatattggaactaccacaagcatggaagatattattcaagagactgattctctttatatctctcaaggcggtccaattcatttaactgggcaagtttcaataaccgattatgataatcctcgtattccgaatactgaacctgtagcaccaacagatgttttggtcacaagtccgtatgctttatttgatttaatttatccagttggaactatctacgaaaacgctgttaatccaaataatccagctacttatatgggtattggtcgatgggtattgtggggtcaaggactagttactgttggttggaattcagatgtaaatgacccaagatttgctatgaataacaacgatttagatatttctggacagccatctcacactgccggtggaactactggagaaatttctgttagtatatcaaatgaaaaccttcctactactcaaaccgatgagaaagtgctaatagcagacaacaacggtccaattgtcgtaggtggttgtcaaattgacccagatgatacaggtccagtttatactaaataccgcgaagactacgcgaagactaatgaaagtcatgtcaatgttaaatcacttgacaatgttcaaccgtcaattaccgtatatcgctggttaaggattgcataatgagcgtaatgagtactaaagcgggtgttaaatcccgctattctgataatattcagtttataccttcgcctaataataacgatgtaatgagtaagcagccatatggtagcttaacaatttctgagtattcaaaaggcattgaattccctaacgtacaatcagcaattaatgatgttagaaattttaccattcgtcctataaattctatagaaattaacacagacggcgtttctccagaaggaatttctcaaaccgacacctggacattcgctggtacagttgttaaaccggatggagctactggcgatgctattatttctgtgttcggttttccggtgacagtaactgttggagatactgccgaagaagttaccgcaaaagctaaattagttttggaagatgcagttcttaaaaattttattattaacacagtttcgcaagggttgactggggctgaacttacagtttcttatattgataaccaaactcatgttcttaaaccggaaacacgttttggcattacagtaagttcaactatcgtatctccagctaaaccgggttatggagcttggacacgtataggaacaaaaaccgaaacattagaaggttcagcggacccggtattgttgcactactttaagagattagcataatgcaaaacaacactctaaaacacatatcagatgaatccaaatatgtaacatttgacccaactaatagtggttttaatgaaaatataactaatgtgcaggatgctttatctgctattagcgttgatggtatttcaggagttccacaagctacagaaaccgtcgcaggtaaagctagaattgcaactcaattggaagtcgatgctggtgtttctgcggatacatttataacaccagcaacgttatcatataggatgcaacgtccagaagcgagtacaactgtacttggtgtaactcaatatgcaactgatgaagaggctatagctggaactgctacaaaccgcactattgttgcgtcatcgttgaaggctactatagacaatgtttttacagttagaacatcaactgaaacttccaatggtgttttaaaaatatcatctacgccagctgctcaagctggcgtagatgacaccacagcaatgacaccattaaaaaccaaacaagctatagctgcagcaactgctcttattccagcttggggacctgctactgaatcagagcaaggtgtagttcgtttggcaacattgtctcaattaagagaccctaatataagagacggttattcagtgtctccatttacgttgaatcagtggcaatctactgagtcaaatttaggtgctattaagcttgcaacatcaggtcatatggatggaggttctgaccatacaactgctgtaactccatatcgttttgcttccactagagcgacaacgggaagagcagggacaactattttgtccaatgatttgaatggagatggttctaaagcattatcagctaactcagcagttcttccaagtaatagggcagcggtatcagctggagtatatgaacatgatactgccgctgataacaagtacatgacacgaaataatcttaaagcttatttacctgttggtactatgacacttgctgcttataattacgacaccggtaattgcttgaaatgtaatggtcgttggttaaataagcatcaatggcctgaattatttgctgtcattggatttacttatggcggagattggggagataactttgctattcctgatatgcgcggcttagttccacgcggatttgatgatgggcgtggtcttgacccagggcgaggttttggcagttatcaagaagatacgatgcagcaaataactgctggatggacaatggatgaccaagcagtaacttcaaactatcctccttcgggagcatgtgaagctagagattatggctctgtcaactatgacgcaagttctgatgatagaaaatggcgcggatttagaatgtatttcgactcagctagacaaactcgagtttctcatgaaactcgccagaagaaccttgcacttaactatgtaattgtagctcgataaagaggaaattatggaattaaagccattaccgtttgttaatggccttccagaagaaggccaacagcgtataaattggattaaaaatggggaggaattaggcgcggcaagtactaaatttggtactgacggtgaactaaatcgtggtcccaatcaagtacagtctaacgtggtcgtaatagatgaaaacgttagaaaacttgcagaagcttcgactgaaatgtcagctcaaatatcaaccatcaatagcgtgttagaagtttctggtaatactgaagctctttctcaaattggtaaaaatacaacagatattggtttattaaagcaatctactactacaatacaagatggagtgcttgaactagacactcgagtgtcatttatcgaagaagatgttggagtttttaatccttctcttgatacgcttcaacgcccaattcgcgatgatttgtattggattaaaaaagaaatggggcaatatccgggccaggatataaatggacaagctgtaaccggaaatgaagcgaccgggatgaaacgccgaattattaataactcttcagcaattaataataacaatgagcgtttatctaaactcgagaaatctttttctgactcagatgtaggttctttaacatctgaagttacaagcattagggcagaattagggccgaaacctactggaactattgaccctatatattctcgtttaacaaaaatgaatggtagtattggcggtcttgctattaacatggaagacgtcatggattccataggatataactccggtgtaacaagcattattggattagtaaatacaaacacttccgatattgttagtataaatggaaaattatctaactcaacgacgggagttataccaagattagatattgttgaagctaaaatcggagacgatgcttctacaaattcaatcaatggaagaataaaaacaaacactaattcaattaacaatttaaatcaaattgttggggctgatacttcttccggattgaggggacaagtagcttggataaatcaaacagtaggtattgttcctagtggttcttctccatctccgacttctgtgatttatagagtagaccatttagaaactcaacaagatgctactaatagttcaattcaagatttacaaattgaaattggtaataacactgagggacttaaaggtcaagtaattcgtttaaatactactattaatggtacttcttctggcgggacagtagaacaaaaaggattgcttcctacggtcaaagagcacgaagaaagtatcaataaaataaacctagatatgcaaaatcttattccagaagccccgatggacggaaaggcttatgtgcgtagagatggtgcatgggttgatattacaactttaactccataatataaagggccgaaaggcccttgaggtacttatgtctattcacgctccacaaaaccctaaagaattaaaagacgctattttaactagacttggcgctccaattttaaatgttgaactcacagaagacatgatttataattgcattcaaagagcgcttgaattattcggagagtatcattatgatggtttaaataaaggctatcatatgttttatatcggtgatgatgaagaactttataagcatggagtatttgatttaagaggctcaaatatttttgctgtaactaagatagttcgcactaatgttggctctattacttctatggacggcaacgcgacttatccatggtttactgattttttattaggaatggctggtataaatggcggaatgggaagttcttgtaacaaattttacggccctaacgcttttggtgcagacttggggtattttacccagttaatgacttatatgactatgatgcaagacatgttgtctccactgccggattattggtacaatggagctaatgagcaattaaaggtcatgggtaatttccagaaacacgatattattatcatagaagtttttacactagcttattcgggagttgataaaactgttggaaacaaagctggatatggatatgcaggtaaagccgaccaatggtctatttctgaccaatgggataatatggacaggtctttgccacaacggcgtgctggagaagacgcaaacgttaagcagggcgcatataataatcgttgggtgaaagattatgcgactaccttagcgaaagaacttctaggtcaggttttagctaaacaccaaggaatgcaattacccggtggagtcactctaaacggagagagattgttagacgaagctgaaagagaaaaagaaagattgcgagaagaattatatttactcgatcctccttttgggattttgattggttaattgagaggaattaaaaatgatgaataataatttattcgcaaagttagaaaataaaaacggatatgaaaaaacaaatgttgctaatattctcaatccgtatgtaaattttaacgggcataaaaacacccaatcattggcagacactcttgtagctgaatcaattcaaatgcgcggcgtggaatgctattatattccaagagaatatgttaaacctgaccagttatttggagaagacttacaaaataaatttactaaagcttggatgtttgcggcttatgttaactcatttgaaggatatgaaggagctaactcattttttagtaaattcggtatgcaagtttccgacgaaataactttatccataaatcctggattatttaagcatcaaaccaatggaactgagccaaaggaaggagatttaatttatttcaaaatggataatagtttgtttgaaataaattgggttgaaccttacgacccgttctaccaagttggaactaactcaattcgtaaaataacagcaggtaaatttatttactcaggcgaagaacttaatccagaattgcagaaaaatcctggtattgaaattccagaatttagtgaattggatttatctccggttaaaaatcttgacggtcttgctgatattggagaaattcaatacgaggaagtcgaccagattaataaagaagcttctgaatttgttaaagaatatgtagtaataaacggacgcggcattaatgacccgcagaacactagcccatttgatgatggttttatgtcataaatataattataaacatactaggcccttagtggcctttggagaaaaaattgttcggacatttttataactcgtctttcagacggtacattttgcttatgggtgatttattctctaatattcaagtagcaagaacccgcgaagacactggaactagatacataaaagtccctgttacttatgcatcaaaagaacattttatgatgaaacttaacaaatggacttcagttaactctcaacaagatgttgctaaggttgaaacaattcttccacgaattaatttacatctcgttgatgttatgtataacggaacatataaaactaatatatctaataggactgctcttcaacaagctgatcctaaaccaactactatttctcaatttaatccaactccatataaaatgatttttgaattgggaatttttacgcggtatgaagatgatatgtatcaaattgttgagcagatacttccatattttcaacctcatttcaacactacaatgaccgaacaattcggtgatgagattgaattcgaacgagatattagaattgtttttcaatctatatcagtagatgaacaaattgacggagacaacatcagtagacgtagacttgaatggtctattatgtttgaagttcaaggttggatgtatccgccgcagcgtgatattaaaggcgaaatcagaacagtttatttggattttcatgctaatacccgtgaattagttccagaaggaattttcgaatccgttgactcagaagttgaaccacgggatgtcaatgttcaggattggaacggagactctaaacaaacttattcttctaatatacctattcctactccgcctgcccctccagggccaagaattgtaaaagaagatgattagtgaggaaaaatgagcgaacaattagatattaccaaactcttagatatcggggaaatccctggtattgatggggaagaaattccggtatatgaaaaattagaattagtagaagtaaaaagtaatccaaatgaccgtaaaccagacttagaagatgattacacggtcgttcgtaaaaatatgcattttcaacaacagatgttgatggacgccgcaaaaatatttttagagactgctaaaaacgcggatagtccaagacacatggaagtatttgctactttaatgggtcagatgacgactactaataaagaaattctaaaacttcacaaagagatgaaagaaattactgccgaaaacgttggaacacaaggtggcaatcaaactaataacattgaaaacgcaacgttctatatgagttctcctactgacctgatggacgaattaggagattcatatgaatctcaagagcgtcaggagaaattagtaaatggaacagactcaaccgtataacgttctgaatgacgctcatccgctgaatgatgccaataaaattgtaattaaacacccaggtcagctagaaagaatggttgaccaaggaattaattggattaaatcgcagtgggacgataagtggtatccagaaaaatttgatgattatttaagaataaacaaaattgtaaagattaaattacagggtgaagacccagataattttcagacttttaaaaataaaaacgtaaaacgttctaggtatatgggtcttccaaacctaaaacgctctaatatcaaagttaattacaccaaggagatgattcttgagtggaaaaaatgccgcgatgatattgtttattttgctgaaacatattgtgctatcacacacattgactacggtacgataaaagttcagttacgagattatcagcgtgatatgcttagaattatgcataaaaaacgtatgacagtttgtaacttgtctcgccagcttggtaaaacaactgtagtagctatttttcttgcgcactttgcctgttttaataaagacaaagctattggtattttggcgcataaaggttcaatgtctgctgaagtactggaccgtacaaaacaagctattgagttattaccagattttctccagcctggtattgttgaatggaacaaaggttctattgaattagacaatgggtcatctataggagcttacgcgtcgtctccggatgctgtgcgtggtaactcattcgcgatgatttacatcgacgaatgcgcatttattcctaacttcatagacgcatggcttgctattcaaccagtaatttcatccggtcgacggtctaaaattattatcacaacgactcctaatggattgaaccatttctatgatatttgggatgcagcaataactggtaaatccgggtttgagccatatactgctatatggaactcggttaaagaacgcctatacgatgataacgacgtgttcgatgacggttggcaatggagctcacagacgatttctgcttcatcgcttgaacagtttaaacaggaacactgcgctgaattccacggaacttcaggtactcttatttctggtatgaaacttgctaatatggaatggacggaagtaactccagattcttatggatttagcaaatttaaagaaccagaagaaggtcataaatatatagcaacattagatagtgcagaaggccgcggacaagattatcacgcattgcatataattgatataacaaatccaatatgggaacaagttggagtgcttcacagtaattctatttctcatcttattcttccggatatcattatgagatatcttattgagtacaatgaggctccaatttatattgaattgaactctaccggtgtttcggttgcaaaatctctttatatggatttagaatatgagggagttatttgtgattcaatgcaagacttaggcatgaaacaaactagaagaactaagcccgtaggttgttcggcattaaaagatttaatagaaaaagataagctaattttgcaccacaaagcaactatccaagaattccgcactttctcagagaaaggggtttcatgggcagctgaagacggataccatgatgacttggtgatgagccttgttatttttgcttggttatccacacaaactaaattcactgaatatgctgaaaaagatgatatgcgtctagcttctgaagttttcagtcgtgaattagaagatatgaatgacgattacgcaccagtagttttcttggatgcagtggccggggccgagtacaacccaactgaacacggtctgtcgtttgtataaatatactaaagcacacatctagaggatttattcatggctttactctcgccgggcgttgagctcaaagaaactagtgtacaaagcactattgttaataatgctacaggccgtgccgctatcgctggtaaatttcaatggggtcctgcatttcaggtaattcaggtaaccaatgaagttgaactcgtcgatttatttggtactcctaatagcgaaactgctgattattttatgtcagctatgaacttcttgcaatacggtaatgatttacgtgtagcacgtgcagttaatcgtgacgtggcgaaaaactcttctcctattgcagaaaatattgaaaccacaatttccgcggctggttctaactatacagttggcgatgttatccgtgtacgtcataatatggatgtaattgaaactgctggtaaagttactcgtgtagatgctgacggtaaaattctgggagtgtatattcctactggtaaaattatcgcttatgcgaaaagtactaaccaatatccagacctaggtccaaactggactgcagaaatagcttcaagttcttctggcgtatctggtactattactcttggaaaaattatcactgattctggtattttgttaactgaacctgaaaatgcttatgaagctattcgtaataccactttccagaacaatttgaaaaaatatggtatgccgggtgtagttgctctttatccaggtgaaatcggctctcaacttgaaattgaaattgtttcaaaagctacgtatgaaaagggcgcggcatctgaactttcaatttatccaacgggcggaactcgagttactaccgcacgggcagtatttggttatggtccacaaactgatgaccaatacgcgattattgttcgccgtgatggagcaattgtagagtcagctgttctttcaactaaagaaggcgaaaaagacatttacggaaataacatctatatggatgattattttgccaaaggttctagtaactatatttttggaacagcaataggctggcctaaaggtttttctggcatcatcaaattgaacggcggcatttctgcgaactcagctgttaccgcaggcgatgtaatgcaagcatgggatttatttgctgaccgcgaagcattgcacgttaacttacttattgctggcgcgtctgcaggcgaaagtttagaatttgcttctactgtgcagaaacacgtagtttctattgccgacgaacgccaagattgtcttgctttaatttctcctccgcgtgaaattatcgtaaatattccgttaactcgtgctattgataacttggttaactggcgtacagcatcggcagcttataccgacgataacatgaacatcagttcaacttacgcatttattgatggtaactataaatatcaatatgataagtacaatgatgttaaccgttgggtgccactttctgctgatatggcgggtctgtgcgctcgtactgataatgttagtcaaccttggatgtctccagctggttataaccgtggtcaaattctgaacgtaattaaactagctgttgaagctcgtcaaccgcaacgtgaccgtttgtatcaagaagcaattaacccggttactggcacaggtggtgatggttttgttctgtttggcgataaaactgctaccaaagttccaactccatttgaccgtgttaacgttcgtcgtttgtttaatatgctgaaaacgaatatcggtaattcatcgaaatatcgtctgtttgagattaacgataactttactcgttcttctttccgcatggaaactagccagtatctgcagggaattaaagctcttggtggtatgtatgagtatcgcgtagtttgtgatactactaataacaccccatcggtcatcgatcgtaatgagtttgttgcaagcttttatgtgaagcccgcgagatcgataaattatattaccctcaacttcgttgcaactgcgactggtgcggatttcgacgaattgatcggaccgcaataatatctcactgggccttcgggccctttggattaaaagtgattatctctaaaactgttaagataaaaatcgttcctcagaacttcaagtacttctctaatcttggatataaactatctccgcttggagcagttaaacatggatttgaatattattctatcgatgttagtgtagagcatctaaaacctggatcaaatattgatgtagaatgtgtatgtgataaatgcagtgagcattatactcaaagattttgtagaaatactgatgtatgttatgaatgtagaagtggaaaatttaagctcggcaataaaattggaactaagaacaaaggaaaaatcttggtttctatgcacggcgaaaatcatcctcgttggaatccaaacaaatcagactacaataagtacgcttctgaagttcgtcgcataaccaatagaaataaatcgatttattcaaagtgggaaaatttcgataaaatcggattatgtggtgtagaaggagcttatcagttagatcacaaggtttctatcaaatacggattttatcatcatataccagcagaaattatagggtgtattgacaatttagagataatcacttgggaatccaatagagataagtccggtaaatctagtatcgatttatgggatttacttaagtgatcatagaattaacttagggaccttctaacgagggtccataaatatatacagatcacacaatttaattcgtctcgatggttctggtgaatctggagctatataaataaacacatagaggttattatgtttcttgatgacatgactcgtgcatttgagtctggcgacttagctcgtcctaacttgtttgaagtagaaattccgtaccttggcagaaactttagctttaaatgtaaagctgctccaatgcccgctggtatcgtagaaaaagtaccagttggttatatgaaccgtaaaatcaacttagctggtgaccgtacatttgatgattggactgtgactatctataacgatgatgctcatactactcgtcaagctatcgttgactggcaggctatctgtcatggtcaaggcaacgaaattactggcggtactccggctgaatataagaaaactgcggttgttcgtcaataccatcgtgatggtaagacagtaactaaagaagtaacaatcaccgggctgtggccaactaacgttggtgaagtacaaatggattgggattcgaataacgagatagaaacgtttgaagtaacgtttgcgatggactggtggttgtaataaatatattcaaacctcaacttcattgtttgaatatgttgcacttatcttacaatttatacggggaataaacttccccgtcattttttagatgctgcaaaactgttataaataagtatatgcttattaccctttcacggagactctaatgaatttcaatattttaagtctttttgctccttgggcgaaagaagacgaacaaaattataaagaacaaattaaaaatgacttagagtctattactgctccaaaactggatgacggtgcgcatgagattgagtcttcagccaatgaagcttcttataacggcctgttccaaaaaatgctaggaagtcatgaacctggcatgaaaaataccagggagcttatcaatacataccgtaatttgatgaataattacgaagttgataacgctgtacaagaaatagtaatggattctatcgtatacgaagacggtcatgaagttgttgctattgagcttgatacaacggattttagtcaacgtataaaagaccggattattgaagaatttaacgaagttttaaattgtcttaattttcaaagaaaaggcgctgatcattttaggcgttggtatatcgattccagaattttctttcacaaaataataaatccaaaaaacgtcaaagaaggaattcaggagctccgtcgtttagacccacgaaatgttcaatatatcagagaagttgtaactgatatggaacaaggagtaaaaattgtaaaagggtataaagaatattttatatacgacaccggcaatgagtcttatcaatgcgacggcagaacgtatgatgccggcacaaaaataaaaattcctaaatctgctgtagtatatgctcattctggtctagtagattgtactggacaaaatataatcggatatttgcatcgggctgttaaaccggctaaccaactgaaattgatggaagatgctttagttatttaccgtattacccgtgctcctgaccgtcgtgtattttatattgatacagggaatatgccttcacgaaaggctgcgcaacacatgcaaaatatcatgaacacgatgaaaaaccgtgttgtatatgatgcatctacaggtaaaattaaaaatcagcaacacaatatgtccatgacggaagattattggttgcaacgtcgtgacggaaaagcagtaacagaaattgatactcttccgggtatgtcaggaatgagtgatatggatgacgttcgttggttcagaaacgctctttatatggctttacgagttccattatctcgtattcctaacgaccaacaaggcggagttcaattcgatgctggcacatctatcactagagacgaattaagttttacaaaatttatccgtgagcttcagcataaatttgaagaaatcttcttagaccctcttaaaactaatttaattttaaaaggcgtaattactgaagatgagtggaatgatgaaataaataatattaagatttcattccaccgcgattcatatttctccgagcttaaagacgctgaaatcatggaacgcagaatcaatatgcttcagatggccgaaccatttattggtaaatatatttcgcatcgttcagctatgaaagatattcttcatatgagcgacgaggaaattgaacaagaggctaagcaaattgaacaagagtctaaagaggctcgttaccaagacccagaaaatcaagaggatttctaatggaaagtcaatttatttccgcatgcttatccaacgacctcgttcaggcacgtaagctttttgaaagcatcatgcttgaaaggactacggatttaattggcgaacgcaaagttgaaattgcgcgttcgtttttaattgaaggcgaagagccaaaagacgaagatgagtcagatgaaaaagataaaaaagaatctgacgacgccgatgaaggcgatgacgaagatgaggataactaatgtttcttatccctgaagaccatgaattagtaatcgaaaatgtcgagactcttattccagaagctcagggccgttacgagtcattatcaaaagcgttaagcaaagacgatataaatacaattgtagaaaacatgattgaaaccgataccgatttagcaattgcactcggttcaattaacgaagatatgcaaattaacgaatttatcgttaagcatgtttcaagtaaaggcgaaataactcgaactaaagaccgagctactagagctagaaacgcatttcaaactacaggattgtctaaagctaaacgccgccaaattgctcgtaaggcaaccaagtcaaaacgcgctaatccgtcaggacaagctcgtggacttcgtaaacgtaagaaagctatgaagcgccgcaaagcgttaggattaagctaatgaaaaaatatggtaaccacagaaaaatttgggaagcagttaatggtcctataccattagatgaatttggacgtagttaccatatacatcatattgacggaaatccagcgaacaatgaattatcaaatttattatgcttaagcccaaatgaccattttgaattacacaggtctcaaggcgatttcggggcagcgtttttattactgaataaccatttatctttatccgcagaagagcgctctgatattatatctaaggcaaattcaaatagatggaaaaattattctgatgctgaacgcaaaagcataataaataaaaatagaaattcaaatatatttacttggtcttctgaaaggcttcgaagagagactggtgctaaaattagtgcatcacactcagccaggaccaaagagcagaaagagcaagaatcaatattaagatcttcttctataaaatctgcttggaaatctagttcatttgacgctcaaagagaacatatgtcaactaaagttgtatgtcctcattgcggaagggaaggacaaaaagctgctatgagcagataccattttgacaggtgcaaatatggaagagcagttattaattgagaactggggtttacccggtgagataaaagaaggaaaacctctaattgaatcagtagattcaaacggagaaaaattgcctcctggcttgtatattgaaggaatattcctccaagctgaagttgtgaaccgtaataagcgtctttatccaaaacgtatcttggaaaaagctgtcgctaattacatgaaagaacaggttcagactaagcaagcattaggagaattaaaccatccacctcgcgctaatgttgaccctatgcaagccgctataattatagaagatatgtggtggaaaggaaacgacgtatacggacgagctcgtattattgaaggtgaccacggtcccggagataaattagcagctaatatccgagcaggttggattccgggtgtttcaagtcgtggacttggttctttgactgaaactaacaaaggttataagattgtgaacgaaggttttaaacttactgtaggcgttgatgcagtatggggaccttccgctccagatgcttgggtgactccgaaacaaatttcggaaggcacacaagattcggcaccagctgccaagaaaagtgctgatgaagcatttaagactctcgcagagagtttaaaagcattataaataataatgtaacttaacaacaggaacatcaaaatgctgaaacaagaacttatcgctgaatccggtgcgctggaaatcgcggtagaactcgacagcgttttcgaatcagttgaactttctccggaagtaaaagctaacttcagcactgtattcgaagccgcagttaaaaaaggtgccgctgacctggcagaaaaacgtattaatgctctggtagaagcagcagaagaaaaagttaaagacgaaaaagaaaaagccgaagaagaagctgaaaagaaaatcacagaagctgcttctaaatttttcgaccatttggccaaacaatggctgagcgaaaaccagattgctgttgaccgtggtattaaatctgagttgttcgaatctatgctttctggaatgaaagaactcttcgttgaacacaacgttgtagtcccagaagaaagtgtcgacgttgtcgctgaaatggaagaagaactagctgagcagaaagaagaaactgcgcgtctgttcgaagaagtttcaatgcgtgatgcttatatcaattatgtaagtcgtaccgttgctgttaatgaagccgttaaagacctgactgaatctcagaaagaaaaagttgaatctttaactgaaggtatggaatattctgatgcttttggttccaaacttgaagcaatcgtagaaatggttaaaggtagtttttcagaaaatgcggcagtaaatgaaagcataaatactgttgatactgaggcaactggccttaatttcgtaactgaagcggtcgctgaaccgtctgatccggcagaaaaagtagtagctccttcaatggcagcatacttagctggtgcaaaacgtctctattaatttaacaaggttatacaacacatgaaaaagaataaactcgtagaaaaatggcagccgctgctggaaaacgaagaactgccggaaattgttggtgctagtaaaaaagctctgattgccaaaattcttgaaaaccaggaagcagattttaaagtatctccggaataccgtgatgagaaaatcgctcaagctttcggttctttcttgactgaagctgaaattggcggagaccacggttacgatgctcagaatatcgctgcaggtcagacttctggcgcagtaactcagattgggccggctgttatgggtatggtccgtcgtgctatccctaacctgattgcttttgatatctgtggcgttcagccaatgaacagccctaccggtcaggttttcgctcttcgtgctgtatacggttctgacccgctggctgataaagcaaaagaagctttccatccgatgtattctccggacgcaatgcattctggtcaaggtgccgctgagaaattcgctaaattgaccgctggcgttgctatcactgaaggcgatatcgttgttcatgattttgctgaaacaggtcgcgcttatctgcaggctgtagttgctgtaactccagatgctggcgctactgacccggctaaactcgacgctgctgtagttgctctgatggaagctggtcagctggctgaaatcgctgaaggcatggctacctctatcgctgaacttcaggaaggatttaacggttcccagaataactcttggaatgaaatgggtttccgtatcgataaacaagttatcgaagctaaatctcgccaactgaaagctgcatattccatcgaattggcacaggacctccgtgctgttcacggtatggatgctgacgctgaactgagtggtattttggctactgaaatcatgctggaaattaaccgtgaagttgtagattggattaacttctctgcacaggttggtaaatctggtatgacccagactgttggttctaaagctggtgtatttgaccttcaggaccctatcgatatccgcggtgctcgttgggctggtgaaagctttaaagctcttctgttccagattgacaaagaagctgcagaaattgcacgtcagactggccgtggtgctggtaacttcatcatcgcttcccgcaacgtagttaacgttctggctgctgttgatacttccgtatctcctgctgctcagggtctgggtcgtggttttgaaactgataccactaaagcagtattcgctggtgttcttggcggtaaatatcgcgtctatattgaccagtatgctcgtcaggactacttcaccatcggttataaaggcgctaacgaaatggatgctggtatttactacgctccttacgttgctcttactccgcttcgtggttccgatccgaagaacttccagccggtcatggggttcaaaactcgttacggtatcggtattaacccgttcgctgattctgctgcacagcaacctaaaggccgtatcgtttccggtatgccttctctggttaactctgttggtaagaacgcttactttagacgagtttacgtcaaaggaatttaagatttaaccttaattaagggaaccttcgggttcccttttttcgtttttgatgtaaaacaatattataaataaaatatatcacaaaaggaaagcgcaatggcaaagattaacgaattactgcgcgagtcaactacaacttctagcagctctttagctcgtcctaatttggttgctttgacccgtgctaccactaaattaatttacagcgatattatcgcagaacaacgtaccaatcaacctgtagcggctctttatggagtaaaatatctaactccagataatgagtttagttttcaaactggtgctacttatggcggacaaatcagtgaaaaagaccgtgaaactattccagaattaactagtaaaactcaaacacttgcagttggtgattattttaaatatcaaaatgtcgtgtataaagctttacaagctaatccgttggcatctacaagcgcaacagatttatcagacgcactgcaagaaggattaatttctcttacttgccgtttagttccagatgctgctaatactgaaaaatttgaagatactgatgttgaaattagtagtgctgtatttgaagtaaataaatggaatgctcctgttaaaactcgtaaattgaaaacctcgttgactgtcgaacttgcgcaggatatggagtcaaacgggttcgattctccggcatttttagaagatttattggctacagttatggctgatgaaattaataaagatgttcttcagtcactgattacggtgtctaagcggtacaaagttacaggcgtttctgataatggtattgtagacttgagttacgcaaatgccccggaagcttcacgcaaactgtatgaaatcgtgtgtgaaatgaattcagaaattcagaaaacaacttcttattcgggtacttacgtagtagcttcttctagagcagccgcattgctggcaggttcaggctggttaaaacatcgtccagaagatgatgaatggttgccttctaccgcgtatggatatctgttaaatgggcttcctgtattctgcgatgtaaatagtcctctggattatgttacagtaggcgttaaagaaaactacggtggaaaagaagtagttggttctattttttacgctccatacacagaaggattagacctagatgacccggaccatatcggtgcatataaggtaattgttgacccagatagtttgcaaccatcaatttcattgatgatacgttatgctttgtctgctaacccgtatacagtagctaaagatgagaaagaagctcgcatcatagatgctactgacatggataaaatggctggacaaagtgatatgtcatatttggttggggttaaactgcctaaaataatttatgaggcgccttaatgcaaaaaatagataatttaattagagaatctactgtctcatcggccaactcgcttggccgtcctaatttattgtcatatacaaaagctacaaataagcgtattttcaaatctctcgttgctgaacagaaaactaatcagccgattgctgcgttatatggaatacgtgttcttaatccagttgataaaatgacatatttaggcggcgctacttatgctggtcaaattggtttgtctgaacgtgaatcattacctttagcttctactccatcccaatcttttaataaaggtgagatgtttttatttgaagatgttgtttttaaagcgttagaagataatccattcgctggaacagctgagactgatatgagagaagttatttcagaagctatcgctgctggacatatcagaatggtatctgacgccgctaacacaaacaagtttgaaaatggtcacccagaaattgccgaagctggatttcgtattgataaatggcaaaccgaagttaaatcccgtaaattgaaaacctcaataactgttgagttagcacaggatttagagtctaatggttttgacgctccaaattttatcgaagatgttttagcagtacaaatggcggaagaaattaataaagatgttcttcagtcattgattacagtgtccagccggtttaaagtacaaggtatatcggaaaaaggtgttttaaatttaacggatcctaaatacgataatgctcaagaccgcgcgcgtactctttattattatatgtgcgaaatgaattccgcagttcagagacaaacatcattttctggtacatacgcagtagcttcatctcgatgtgccgcgattttagctgcatccggttgggttgaaaaatcagaagaccaagatgagttagcatatggtgtattaaaaaatggtttgccactctatgctgatgtaaacagcccatgtgattacgttattgtcggtgtaaatgctgatttaggtgaaggacagacggttgcatcactgtattatgctccgtatgttgaggggctagatgatgttgatgaagaaggtgatacttctgttggtgagttcaaagtaattgttgacccagatagtttacagcctactattgctcttttagctcggtatgcattaactgctaatccatatacagtagctaaagatgagaaagaggcacgagttattgatggtgctgacatggataaaatggctaatcaaagccaattaagttcttatcttggcgttaagttaccaccgcttgagaaataagaaaaaagggccttcgggcccttataaactaaatatatcaaacttcggagcatactgttcttgtagctcttcgaaaactttcttatcattcaaatttgtacaaggtattccgtgttgtcttgcaatacgagtagctatacgagttccaccctgttcttccccatttcttactggacaccaataataaacgcggtcaaccggtgacatacaatcttctcccaaaacttgcatcgcatttcgacagaacaatgtctttataatatctcgttcagaatccagccctggataagcttgcttagctttgacgtaactctttatacgagattcattacttaaatcagaccagcaaaccacgccaataccggtgtggcaatgattaaacccatcataaggaataatcctaagcgaaagatgtctatcatatctgctcaaccaagcttcgtctgagcctggagctccaccagaataagagaaatgaccagcctccgaaaaggccaatcccattaaactcattaagtcaagatactttcttggtgcttctcttgacccgattaatgctaatcttaacttacgatttctagccatttttctctgatagtattactcacaacagtctgaagttctcgttttacgaggtccggattatcagcatgaacaatatcaacaccttctcgtttagcttcaataaagatatcctgtattgtcagacccataaccttaccgaagtctttagctttaacttcgccaatatgactaatcacgttagatacacggttccacgtagagtattcacagaggttcgccagaacggtcgagtcgttactagttaatacagctttaggtttaattggcttatcagattttttcttctcagtgaatctggagttcttagatttaattgcaatgcgggtgccattattaaagaaatcaggataaactggttttagtacgtaaccttcagcaacattatcatctgtcactaatgggtcaaatacacacaaattgacttctttcaaatcatcttttgctgcttcgttataagctttgattacagatgtaaaattatttggtaattgaataagttcatcaaaagaaccacatccaataaatggagccattttaaatccgaattcgttacagaacgaagtcatcatcatatcgtccatataaagaacattaccattttgagtatttactagaatgtcaaatacataaaagtctttttcaccataatcaacttctttctgaattccttgacccgcgaactcaccaaaaatttgataagaacttactgaaccaagttcattcgcattcatgcatcgttgaatagttttaatagactcgtcataattcttcaatataatttcgtggccatagaatgattcactaggaagaatgggaccactgcgcttgcacgcagaaactgagtctttggatacgatgattgagaaattagtgccatgaattttttcacgagctacccacaaaccgccatctttaccttcaaaccgaattttagaaataaatttattgttgtagtggttttcaagagaggagtacttcttaaacattttttcaccattataaatagattaaatagagaacttctattcttttccggcagagtgtaatggttactagatattatccttgaaaccattatacaccataaattcttaaagcattttaccacgtaatcataatatacgtgccggaaatttggcattggttccagtatgccgtaaatccttcatcttttaaccatgacatgatatttcctttgaaattagtttcttttgggtagaaaaccagttgagtcttcccatttttggcagcattattaattgattcttcaattagttctttttgcttaacaaatccttcattgttataagaatctgctaagttacggtatttttctgataataacatttagacctcgcttggaataatacgaatatcgatgaaactaaaatcttttgattgtcgtaattcaatctgatttttatcaccaacttcaataaccactatttcatttaattcgatttcagcgatattaaaagcaaatcggtttttaattatgttataaagtatcacaagataatgttcattgtaaaacgaactactatctccaaaagaatacgataattttttcataaaatctttgattgaacaccatttgttcacaccaagcgatgctggagcaaacaaattttcatacagcatataatcaatgatgtcagtaacaactagttccgagtctatcagcacttgagttaagtttttcatactattcctctcattttattaacaagtttcttacgacgaatattaacatcaatatttgataagtgtttacgataaacaatcttctctttgtttttcgctttatctcttgcggcctcaactttagtaaacattaaagccgcgtcaaaagcacttattccacctataattttagcaatatctttaaatgacttgccgttttcatgcatcatgtgcactttaatttctgttttcataatcatctcttcgttggaattgaattcattgtaatcatactttagaaaagcaaaaagggccgcaagggcccttcatgttatggataaataataccagcttctaaagcagaacgatgcacaatatatccatttctagattcctgaacatcaacttctggataatcattaagcatttttgctaataccgcaagatgacaataatatggtgtatcagcttcggtagctgttttccaatctttaccttcttcagtgagcttctggatggcatccataacccaccagccaacccaaatataagctgaatgacgttgtggaagaggatgaacataaatcagtgggcattcttccggaacaacaggttctgctgcttcaacagtaacagacacattaccggttttaactgaatctgtaaatccagattgtgacacagtaacttcacaagcatatgtcttcaagccttcagtagattcagatactactcttgtggcttctgtcgagttgtcatcccatttatacgcaagagtagcaccaggaatagcagggtttatagaagctgtcaaattgaacggaacatcaactgttgcagtagacgggctcgaaatgtcaacagtgaactcagggaacgtattattttcaacagtaataactaccgcgtctggcgttatagtttgattgttataatccgttgcagaaacggtaattaaacaagtcaaagaaacaggtcccgcgacagtagcagtacgagtaatatctttagtagtttcaccagtagaccacaaataatcaaatgttgcaccacttggggcaccttcaacatttgcagaggctttatattcctgtccaactttaaccgttggagtagaagttgataatgtaccggttgcagtcatagttttattattgactgtaatggtagtttgagcttctgcggtttcaggttctccttcggctggggttgtggttgccacaaccttaactactttcgaacccgccggactagccgcgatgtaatccatggtagcaataacagaactctgtggaaccccatcaacggtccatacaaaagattcggtacctgcagcagccgcgccggctccagtagcagtaaatttagtcgttgctcctatgacagctgtaggcgttaaaggggcaattgttacggtaaatgccatatttcctccaaggagcttttgctccttttatttttgtttagtataagcgcacaaaagatgaatttcttgtttcgcggataagaataaaaccgtcgcgttgaatataataaatcaaacttagaagagtttgatgcgcggtagcatgtggaaaagacaatggtctttctttccagtcaggattttcagaaatccattgataaatccaccaaggtaaagtataataacctggatttttacccatcagttgaaaatgaggattaaatgattcggggagagaaaagcttttattttcaacaaccgcagtatcatcaataatcttcattatttcatcagcagaggcagtttctggaatacgaactgcatcttctggaatttctttggttttcgccacttctaaaacagtaatggtttcttcggctggagagtcaataataagctctttgacttctactttatcttcgggttgaaccaaatctttttgttcttctttatcgtcgtcaattaagtcagaaattgaaataccatcattatcttctggaagaggagcatcagcgagcttttcaaattcagcttctaaatccaaaagcatattttcgaatgacttagttttcttcaatttaataccaaattgagatgcgtactcatcgagcttatctttagcttctttcttttcaagtacacggatttcttctatataattcatgtctagcatagtgttgcctctttagttatataaatatacttgtatttataacacggagatgtgaagttggaaaattgcaagtatcattatgtttatgaaataaccaataatattaatggtaaaaagtatataggaaaacactcaaccaacaatttagatgatggatacgtgggttcgggtgtttttataaaaaaggccatcaataagtatggattagaaaatttcactaaaataattataaagaaatttgatacatccaaagaagcttttgagtttgaagccgagatggtaacagaagatgttgttaaaaaccgaatgtattacaatgcgaaaccgggtggttatggcggaatttatatgacagaagagattaaaaagaagatgaaagagtcatctagaaaaagatatctcaattcgcctggcacaacattgggaactacgtgttatacaaatggaaagaaaaatagatttttaaaacctggcgataaaataccagatggattttatattggtcaagttattccaaataaaaaatgccgaaaggggtgcgccgttaaaccaactacaactggaacattttgggttaataacggaacaataaataaacttatgcaaccgtcatctgaaatcccagttggctttgttaaaggtcgcttgatgaagcgtggtattaatggtgaattcataaagggttaattgtggatataaaagttcattttaaagactttagtcatgtttatatagaatgtgaagaatccattctattcgaactaaaagactatttttcattttttgctgatgggtataaatttaacccaaagtttcgttatggaaactggtcaggtaaaatttatttattagaccataaccgtcttttacctttcggtttagttggtcaaattaaaaagttttgtgataactttggttacaaattttggattgacccaaaaatttttgaaaaagaagacttgtcccgtaatgactttgatgaatggttagggaagctccagatatactctgggaacaccaaaattgaaccgcattggtatcaaaaggatgctgtatatgaagggatagtgaaccgcaggcgtattctaaatttacccacttctgctggtaaatctttaattcagtgtcttctttctcgttattatgttgaaaaatatgaaggaaaaattttaatcatcgtcccaacaattgcgttagtagaccagatgattaatgatttttgcgattaccgcttatttggtaaacagcacatgcttggtattcgatcgggtacaaggcgcgactcggatgctatgatttatgtcgcaacctggcagactgcaataaagcaaccaaaagagtggttttctcaatttggtttgatgatgaacgacgagtgctttgatggcgacacattaatcaaaacaccaaatggtgatgttaaaattaaagacatcaagcctggtgataagatttattcttataatgaagatacaaaagaagtatttgaagatgaggtagttaaacttcataaaaacttaactaagtcatcatctgaaaaaatgtatgaactcgaaatggacgacggtactatcataaaagtaaccggtaaccataaatttttaacaaacagaggctgggtcagagccgatgaactcactgaaaatgacgacatagtagaatggaaataaaaatattacgcggcccaagcggccgcattctaactaagtctatggtcaaaaacggtacaataaatgctttaaaagccatgtacacgtggtgtatagataaacctagtgctttggtgtactgttatgaaaataattctattcaacctaattgtaaattctgtggcgaagggttaaattcaccgattaatagctattgcaatccaagatgtcaaatgaaacaccttggcggatttcctaaagactgtaatgttggaaagaccccgtggaataaaggtcttaaagataattctagacattgcagtatagtaaatccggaaaaatgggaagcagccaagcataaaatatccaaagctaattccggagaaaataacggaatgtatggttgggataatgaagaccagcgacggcgtcaaagcgaaactataaaggctaaaatactttcaggcgaatttactcctaataccaataacagaagaacttcatttgatgttgtgtatgatggaaaaaattacagaagcagctgggaagccgcgtacgcttccataaacaaaactgctgtacatgagagaatccgtgttccttatattggaaacgatttaaagaaacacatttatatttcagattttttaaatccggaaacaaacgaaatcgtggaaatccgtccggcgtctctatacgatgacaatcatccaaaaattgtggctattaaaaagtattgtgattccaatggatataaatatactcatattgatatagagtatttctacgcgaaccgtcatttgatagattacgatggtttaggtgatgacacatccaggaaaattacaaatgcgattaaaaagtataaaagaaataaaaaaacctgacgttgtatacaacttgcatgttaaaacaaatcacaactattttgcgaatggtgcattagcacataactgccacttagctaccggtaaatcaatttcaactattgtttctgggctgaataattgcatgttcaaattcggtctttctggttctctgaaagatggtagagctaatatcatgcagtatgttggaatgttcggtgaaatttttaaaccagtttcaacatccaaattaatggaagacggtcaagtaacagaacttaaaattaattctatcttcttgcgttacccggatgaagccacggttaagatgaaaggtaaaacatatcaagaagaagttaaagctgtcactggatttaaaagaagaaataaatgggttgctgctcttgccaaacgattggctgataaaggcgaaaatgcttttgttatgtttaaatttactgcacatggtaaagagctttatcagatgattaaagaccttggacatgaaaaagtttattatgtttcaggcgaagtgtcaacagaaactcgtaatgctctcaaagaaatggcagaaaacggtaaaggaattattattgtagcttcttacggagtattctctaccggtatttctgttaagaatcttcatcacattatttttgctcatggtgtaaaatctaaaattactgtattgcaatcaattggccgagttctacgtaaacatgcgtcaaaaagcttagctacagtttgggatttaattgatgatttaggtgttaaaccaaagtcagctaacacaaagaaaaaatatgttcatattaattatttgttaaagcatggtttagagcgtattcagcggtatgctgatgaaaaatttaattacactatgaaaactatcgatctttgaggaaactatggaatttaaacaatttatacgtgaagcaagcatcggttcatttatgaataaaatcgctcaatgccagacattagctggattagatgaactggaagcttattacaagaaaagaattaaaacaactgaaatcaatgacactgatgatatctctattcgagacgctttggcgggcaaaagaatggattttgagtcagatgatgagtctgaagttgaagaagatttctaatacaaaaaagccccgaccacaaggaaggggcaaaaccaacaagttggctatcaacactagactaaattagcaattgagtttcgtttaaatgctcttctgtattttcagataccggcaattcaacgacataattataacacggcccaggatgaacaggacctttatctgtgtgaacaaccaatgccgaatcgatcggttgcttacaaacagcgcagatttcagacattgtttgtctcctttagttaattacatatctatttatacagttatttacccgcctcaaactgtctcatctcaataacatgcttaattccaaaacctctggatttaatagcatccattgctcctgagcaaaattcaagcaaaattccccagtattgtaaactagtgtcaattttaagaacttcagaatcagctcccaaaactgttttcatttcagacttttcgtaacggtccatgctaaaatcatctccatcaccacgtcccgtgtagtagtccaatttctttttaagagctgattttttctgtgcttcaatacgaagcatttctttacgaattgatgaatgcttatttaaccattttccatacaaaactggattattagcagcttcgtattgtaatttagttgagtctaagattaaatcttgtttcaactcttcttgtaaatcctcgagtttcaattcattacctctttcttgttcatttgtaatcttgttgttattgattgaaaggaactaagaccattataaccattaatcactgaaagcgtatcatctattacttgaagcaagttgtagtttaatctgctcgatatcatccggattatcaacaacgctgaattgaacttccactataagcgtataatcatcataaactggagtgacattaactgctaaatatctaatcctcggctcgtatgctcgtatagaagattctatatttcgttgaacagtgtcagcagtcaatggcgtcatattttcaaataattggtccgtaagcgagcaaccaaaatttgggtcaaaaggcctgcttccttttctggttgttataatacctaaaagagagtttttaattgcccgaagcccgcgtgagcgcgctacgtctttatcccagtccattttaaattctgggtcaatatctgaatacaaattttttatatcagccattatattaacttaaagaattctttaagcccctttatgatatgaacatgattttctccgcattcactgcatttaaccggaactgcgagttgtactgtgggttttagcaggagatttttaatattaatgatatcgtcttctgttatagcagaatataaatcttctatttctctgctgtttaaatcttcaataccgatagtttcgccattaacgtgaatggtttgaatacacgacgatatcatagaagcaatgttctcatcctcgaaaagatttggatatctgagttttattttaaaatgtctcaggttataccagagctcctctggttcatcgatacatgcgaatgtaaagttaatcggcacttgtgtttcatgtccgcaggaacacacccatgtattctggtgattaacctcacctaaactgtgagaccacaaatttactaacaataattcagattcttgcttgttcaaatttttagcatttgtacagtttttaatcaaatctaatacgttttgctttacatgaccattttgtttagctatgattagatttttatattcttctagtgtaaaagccctgcactcgatggtcttttcaccgattaggacttcaaaattatagtcatagttcatgatttactcctcttattactttatttataaatattaataaaggagaccacaatggctaatatcatacgatgtgtgcttcctgatggtgttcaccgtttcaaaccgtttacagtcgcagattatcgagattttctgcttgttagaaacgatttactgaacaagtcatcggacgagcaaactaatatactaaatgaacttttaaatgattattttcccgaatttcccgaaacttggagacctcatatttttcttaaagtttttaccgggtcaataggaaaaacaaaaataccaattgcgttttcatgcccggtttgcgaaaagaagaaacaaacattatttgatttgcatttgaatgatttaaaatctccggaagtagaagttgctggtattaaaatttattttaattttcctgataaattttatgaagataaagcccaacaaattaatgataatataaagtctgttttatataacggaacagaaattttatgggaagatttatcagaagacgataagttacaagttatcgacgctatagatattgacacattagaaaaaataataagtcaaatgacaccaatgagtttaactcttaaaatgaaatgctgtaaaactaccactataaaatatgaagattttttaaatatattttgtcttctattaaatccagatgaagtttttagtttttatcagattaaccatatgctggtaaagaaccaatacgatatgaacagcataatgaatatgatacctgtagaacgaagcatagctctttctttggtagagaaggataatagcaaatgataacacaaattccaggttttcccaatttaagtataaaactttatcaagattacgattcttggcaaaccaaccgatacgtcgagcttgctgctaccgtaataactttaacaatgcgcgacggtctatatggtagaaatgaaggtgtattgcaattcttcgattcaaagaatttacacactttaatggacggcagacaaataatacaaatatccgtagctaattcaaatacaaagaaagtgcaaaacagaatttatggatgtaaacactatagcgtatctgttgattccaaaggagataatattttagctataaatttaggactgatacatgaaatagaagacctaaaatttagcagatgcttttttaatgatgctggtgaatcacttaaagaaatgatcggtgtcatatatgaagataaacctttaatagctcctgctatcaacacaatcaacacatacgttcctcgggttccgtggactagcaatataacaacttataagcagtacgtccgagatataggtttagctgtagataatgaccaatttgtttttgtatgggaagacatttatggtttaaacatgatggactataacactatggtaaatcaagagaccacaaaagttgtagttggtgaacctaggacaataggacaatttgttaatgagcttgagtataatttagcttatgattttgagtggctaactaaagccaactctcatgtacgagaccctatttttaacgctactatatattcacactcatttatggataatgaaattccaagaatagtaactggagatggtagcaatagtatttttgtttctaggtctggtgcgtattcggagatgacatacaggaacggatatgaagaagcagttcgtatccaaactatggcgcaatatgatggttatgctacctgtaaaatggttggagattttgaaatgactccaggcgataaaataaatttcttcgatacaaagagacagttcagagctgatttttatatcgatgaagtcattcatgaagtttccaataatcaaagtataactactctgtatatgtttactaattctcgtagaatagagaatgttgaaccaattaaggtgaaaaatgaacttaaacctgatacttccaattaagaaaataaaatgcaataataaagaaatatctattcctaagctcggtttaaaacatcatcatttaataaaagaagtgagagacctatcagaaaatatggggattttgttggattcaatccacccaggcctcactgcagcagaatccgacttagtgtgtcttcatttattagaattcaacggtaaattaaagtctactgtaacaaaggacggttttacttacaatataaatgacgtttatatatgccaacggttagaatttcaatatcaaggaattacattttattttcgttctcctgagaggtacgaagtttttaccacagtagataaaatgctgtcttcttgttttattaaaactaatttatccgacgaggcacctgatttccttagaatgccagcatttgtttcaaaatgggcagatgatataacaaacatgatagcaattcctggtccacatgaacctatcaaaggaacctctaaaatattagggctatttgaatgaaaaccgaaaacatgacatcctttagacgccgcaaagttattgctgattctaaaggagaaagagatgcagctgcggctgcatcaaatcaagttgaatcattagattctatagggtacaaattagactcagttcaatctgctacggagcttacatctgaagtaattgaacagaaatccaatgatattatatccgctgtgaatgacactacagctggtgttgaattaacagccgaatttgctgagaatacttcaaagacggtaagagaattaacagatgtaacatctgcgataagcgacaaaatatctaaattgacggatatgcttgagcagaaaatacaggcagttcaacaaaaatttgttgatagctcaaaggttaccgatgatactcttaaagtcattggagacagcattcctgagcccgttgaaagtaatttacctgcaatcccggaaaaaatattcgataaacctgaagaaaataattctccggatgctgatttctttccaactttaccaagtaaagccgaagaagttgataacaaaaaagactccgataaaaaaattcttgacactgaaaatttgttaaaagatttggtcgggactacaaagactgggttcaaagcaacagtttcaataacagataagatatcaaatatgttattcaaatatactgtgtccgctttagctgaatcggcaaaattagctggaactatttttgctattgtacttggtatagatttgcttagagcccattttaaatattggagcgataaattcagcagcaactttgacgaattcagtcaatccgccggagaatggggtagtttattgcaatctgttctgggctctttacaagaaattaaaaaattctgggaaaataatgactggtccgggttagctgtggcaatagttaaaggtcttgctgatgttctttataatttaagcgaattaatgtctttaggcatttcaaaaatatccgccgctattttaagcgctcttggatttgataatgctgctctatcaattaaaggtgctgctcttgaaggatttcaagcaagaacaggaaatgagctgaatgaagaagaccaagatacattagctcgttatcaaactcgccgaattcaagaagggccggatgcttttgataaattttccgaatataagactagagcttttgattttataacaggacgagataataaaaacaccacaacaacggaacaagaacgggaagctgaagttaagaagttaaaatctcttccagaagaagaattaaacgagattaataaaaagagcaataacgctagagccgctttagttagatttgaaaaatatatgggtgatgttgaccctgaaaatgcgactaatattgaatctctggataaagcttacaataatgttaaatctcttgttaatgattctgaactgaataaagccccagctattaaaaaggaattagaagtacgtttacaaaaagctgaagctagatatcagaaaattaaaactgagtctaagcctgaacctgctgccccatcagcctctgaagacgtgcagaaagttcaaaacatcgaaaaggcagaacaagctaaaaaatcagatgctaatcagagcagttcatctagcgttgttaatgcccaagttaataacgtaaataatagtagaacaattcaaactattaatcctgttactgctacacctgctccgggtgtatttaaagcaacgggcgtaaactaaggaaaaattatgattgttaaagagcttaaagatacggctaaagaactatggaataaaggcgaaaaaatttctgctggtcaaagttctcaatcatctaaaattaaaagcactgtaactgtacaatacccgtctgagcgttcagctggtaacgatgttacaggcaacttgagagttcacgatttatataaaaacggtcttttatttaccgcatacgatatgaattctagaacctctggtgatatgagaaacatgcgtctgggagaactcagacgtacttcacaagatatagtcaaatccgtgaccggtaaaaatactaaacaggttgataaaatacctgttgctaatattcttttgcctaggtctaaatccgacgttgattcgacttcgcataaatttaatgacgttgctgattctttaatttcaagaggcgggggaactgctacgggagttttaagtaatgttgcgtcaactgctgtatttggtgcgttggagtctgtaactcaaggactaatggctgataataatgagcagatatacaatacggctagaagcatgtatgctggcgctgataaccgcacaaaagtattcacttgggacttaactccgcgttctgttcatgaccttatagctattgttgaaatatacgaatattttaattattattcttatggtgaaactggtaattcaacttttgcaaaagaagttaaatctactttagatgaatggtataaatccacttttttagatactttaacacctaccggtgctcctcaaaacgatacagtatttgagaaaataacttcatttttaagtaacgttattgttgtaagtaaccctaccgtgtggtatgtaagaaactttggtaatacttctaaatttgatggtaaaactgatatttttggaccgtgccagatacaaagcatacgatttgataaaactcctaacggagttttcaatggtctggctgttgcaccaaacttaccgagtacttttactttagaaataacaatgcgtgaaatacttacactcaataggtcgtcaatatattcggaaggtttttaatgtatactttagatgaatttaaaaatcaggcagctaatatagacttccaaagaacaaatatgtttagctgcgtctttgcgactactccttctgcgaaatcccagcagttattggaccaatttggagggatgctatttaataatcttccactgaataatgactggcttgggttaacgcaaggagaatttactcagggcttaacaaatataataacttctggtacaagagatttaactagaaaatctggcgtatccaaatatctcattggagctatgagtaaccgagttgttcaatctttattaggcgaatttgaagtaggaacatacctgattgattttttcaatatggtttatcctcaatcgggcttaatgatttattctgttaaaataccagagaatagattgtcacatgaaatggacttcatgcacaattctcctaatataaaaataactggcagagatttagaaccgttaacagtgtcattcagaatggacccagaagcttcaaattatagggctatgcaggattgggttaatgctgttcaagacccggtaacagggcttagagcacttccaactgatgttgaagctgatatacaagtcaatttacacgctcgtaatggaatacctcacactgttattatgttcactgggtgcataccaattagttgtggagcccccgaattgacatatgagggagataatcagattgctgtttttgatgtaacatttgcttatagagtgatgcaggcaggcgccgttggtcgccaggcggcgatagattggttagaagataaaactgtcgattcaatagataagataaatcccgatttatctctaaacggaagtttaagcagactttcgagattaggaggagctggcgggggtatttctaatattgtaaatagagtattctaaaaaaggggaccaacgcggtccctttttttagttaacgaaatacaacaaatcctactacattagatatttgattgtcctcagcgataatactaattgcacacggaacttcaccaactaaattacccgacaatacgccagaaatatagttatcagcttgtccttcttttaccccaaatctcggtaaagggtgtaattgaaaatttttgtcagttccatacaattcttgaagcagttcatatgctgcttcattaaattcacgcgaatcaggtttagcattttcaattacaatttcagtgcttgcaatacgtaaaatagatttcataatatgttcctcatgttgatttagtataattataacacatccttccatggatgtaaacggtcaatcgacaaaacgatttggtgtatcgtcgaggtctataaacgaagctaaaagagataaaacactggtctcttcagatattttcgcggatttgacaaaattaccaaaatcaatcaatccagcgcccaaactccctcgttctgtatcatctgaatactctataacttcaccagtttccattaaatggtccccgtcataaacaacagtacattcagaaagttgttctgatgtcatgatttcagcttgaatgaatttttgattgctgtattccattccgtcattatatgaagcatctgtgattttgttaatttgaaccatcgtaccacgaggtaaaatgatttccatttcactagtcatgttacttaaatcacctggataaatcacattaactttatgagcgccgtcaatagcccaaccaacattaactttaatacgctcttgtttaactgggtccattttagactcataatcgtctaatttaacgtcttcaatgccaccgtcaatattcaatacattcctagtagcgtcagacgtaaatgcgacaccctgattacctttccacccgccaaaaataatcggagctaaagaagttgatacataattcctaaaatagaatactctgtttttaaccaaagcttcatatatcggtttgcgaattgtttgagaacgccaaattgtgattccttctggtagtctatcgcctcttaaaaaagctttatctaaactagaaatcgcctttttgacttctttttctgttaatgtatcatagttgtcgatagtgtatcgaccgagtaacatattattgatatcagaatacgcagaaccaacatattcttttatgccctgacgctcttctttagtatattgctttggagtgcggtacatagtaatatcacgaatattacgagatactgttttagcataaccttgtaatagctcttttataagcttaactctttgcatgttccatgcgcgttcagcatgttcttgcttttctaaagcagacataccataaggagctgattttatttgagcctcaatgaaattatgcttattttcttctaatctgtcaagcatagtctgcacaaatgcttgcatacttttgacagaagttaaattatcatcgccaattcgctttaaaagctccttttgaacatttttttcttctctttcaaaatccgtaaatgtcttacctacgattttggaaacatgatagacaatttcacctgatgcagtaagagctgatacagcctttgaagcttttgagcttatagcatttaaaatctctttggattctggaattattttcgccgtagcaggtttactaaattcagcggcggtagcttcatagcgttcaaaatctgggccttcaaaacgttcagtttccaaagactgtgacatcgcgacctggcgacgagaaattttagctcgagaaattacgctttgaactgaacgagtgtcattttcctttgcaatagatgaagcaattgccgtgtttttggaaacttgattgccggttttaacatcaacgtaaacttcgcctacagcagtatcaaccttcttgaactcatccgttgatatctctgggacccctggaacggttgagaggtcaatatttttacgatgaataagaatatatgcgtacttcttatcataatcccagagttctttgagtataacgtatttacctccagtgcggctacggactaatcggtccatgataatctggagttgacgggcttgtccgccaattttagatttagtgatacggaacaacactgcatcaattttatattgacgcatagatgaataaacaatattgaatacggtagaaatagttccgattgggtctggacccaagtttttcattttaaccaaagaacctttttcgctcaaagataacataactacgtgcgccatcttatcgccaggcttaatatttttattagtatcgccgcctgacgtataagtacacatacgaaaagccggcgtcgtaccttggtcaatttcgccaatagcaaaaatctgtggtattttcgtttttggataaagatttgtaaccggaaggtccaacttatcttcatcaaaaacttcatttaattctaacataaacagtcctctatagtttattatatttatagacgaaaaaagccccgaagggcttaatacatttttgttacttgtcgccagattttaattcccggagcttcttcaaatgatactacatcttcaaaatacttatcggctatatcccaactattaaattcgagctggtttgttgtgatagctacattaccattgtctgatagaacagtaacttctaataaaatatcgtaactcataatccagtaacctcatggaaatcaccccagatatctgcaaacgtattagcaacatctttatcacgccgcatctttattgcaataggaagaaacagtttaacataatcagttcggccttctgccgccaaccaaccattacattcactttccaatacgcttccgatatattcttcttggttttcccaaatgcgtgtacggtcaagttcgtgagcgtcttttccgggcttgtcttttaagcctgaaccggctttaactttaattaacccgcattccgattcaagataaaaaccaccagcttttcctggatgtttactatgcggataaattccaacaatacgaaggtcaacagtaattacttctttaaatttatattggttttttgaacgagcattctcccaaaaaccaccaatattcttaagaataatcccttcgagtcctttatcaacatatttgcgataaacgactttagcttcgtcaagattgtggacgatatggttctcaatgagaatcatctgagagtagtcttgaaccattagttccagcgctcggaaacgaacatcatacgcaaaaccaggttcttttccatcggaatatactacatccaatggaacataatcccacacttgaaacttcatacctgcagcttctttggctgagatagttcctttcagagatttgttggctagcccgtttgatgcagttcgagattcgttttcttctgatttactcaattcagtcaaatcaccgaacatatcctctaatggtccagaaggttctttcacagcatgataaaccaactcaccgtcaatcataaccccgcccggatgacgttcacgagcttctttggtcatatcaataagttgctgttttaacaaatctaaaccaagatattcattaccagcacgagacagaatttttacatcatctaattcatcaccacgaacttcggcaaacgcccttgcgccatcagccttaagctgcgcaaatgcagggaatttaatattcttaagaatatctttttcgttataagatgaagcaagcatttgtggttgttctggaattaatccagcccaaactttatttgcaatagaacgggatgcaccacaacgaaggtcacgaagaagaacctttttcaatacttctgcatcagagacattcaagtctgcgatataacttgctaattcaacaatagcagcgttaccagtaatttcacgagtggctaagttatattcaaggaaatctaaagcatcatctaaagttaacattccaaaacattgtgtacgctcaccagcgccaggccacttcttgatgtaatattgaagttttccgtcataagtcattcgaaatacgcgctttaaaacttcattatctttattgcgacgaatgattgcttctttttctttggtagaacctatggctgcaatttcattaataatatctaaaatcatattcatcttctctgtttataaggatacaactattttatcatatccagattaaagcttttaaatattcacgtcatgccagcttttgacgtaattgcactctaatttaggttcatggttcctttcaccgcgaggcatgtaaaacaccttcgcccgagaattatactgtttgaacaccgctgctgcggaatcagtatgtttgcccaggtcatcaacgtaacaaacgacacgctcaccatatttttccattacctgggttaatagagcatccttagatgcattataatcacatactaatatttctttaaatgctcccggaaaaagcgcatttaaattaaactggcgatttagatgagcatcaactgagttacctaacgcagtaaccgcaacgaaatcgtaagttttctttaattcgttaacaactttcaatgcatcttcataaactgatagataccgtataaaatctgagttgttatattttaacaaaagtttggttgcgaattcttctgaacaattaaaaagaaccccaggagcaataaatttttcatcaaccatcatttcaagaatatgatttaatggaagattatatttctgggcaaaatatggtaaaccgctttgccacttaaccaaaattccatcaacgtcagtaacaattactggcttttcttgttccaaaatcatacaatttctcttaacattgtaataggagaaagttcttgttccatgtgcacagaccgatttccgtcaacccaatcaatttcatatgcagctaaaacaccgtacttagcaataggtttaatcacatagcagatagttccaggaatgccagatgatttaagttgtactgtctgaagttctttgtattttgctttcatactttttcagccttaaccaaaataacatcactccatatgccaaacggagggagagcttccttaactttgtggaattctgttgttttatcttcccagaaccatagttttatttctctagttacttggaaagaatccatcgcccgacctgacggtgaaacatcaattacatcaaatgttaacttgtaagttggataaagacgcttaattaacttagtcaataatttcataatacaaccacccatgacggaatttcaaagaaatcgggagcatcaatttcatactcaaccgaatgattacgcacacgtacttcaattacatcaatcattgggcaattaatcatacgttcaaatttggcagctacagatttagcaacagatacaatcattttcaattcctcaagtgttgggcccgaaggccctttaattaaaattcaatttcgttagcaacaacatcccacagtgcttcaagttgttcttcagtagctcttttaccaatagcccgccgtagataaagcttgaccaaaagagcccgattagttttccaagaaggatgagtacttaaatcgcgccggaatatttcatcatgaaaaccaacctttatttctgaaccattgaactgaacatctaaagttggaccattttcaaaactgatgaaaacgtaattatcttcccagtacatatcaatctgtgctactgtaccgttgttatgttcccacaataaagtggtagaaatatcccaaccgttgttttgaacgtatttacgtctgaagttagtgaagtccatgttattctccgttgtatctgtttgtttattgagtttatactataacacatacaacggaggatgtaaacggttagacaaaacttattttgacatttttcttagcttctaacttaagctctgtgataaaaacatgataattatcacttgttgacatgcataattcttctgcaattttcttggctttttctaaagtaaagaggttcttttcgttttctctaaaaacgactgcacttccgcattcaagagagcgaacaaccatgaatgtttcaggtccttcagtctcagatttaaactcaatttttccttgcttcacttcatcaaaatcaaacagattccaaccgagccagtcttggccgcaatctgcgccattataacgcctgtccaagaattctacttcagtgatatacttcccaccgacagatacttgagtagctttaagccaatcatttccaaacaagcatttgattttggtgtcaaatgagtctaaatcgccaccccatttatcagtcatacggaatacatcaccaacttcaaagcatgttttcataatttcctcattaaatttaaactaagacagtattggttattgagtttatactataacacacttttctgttaatgtaaacggcagactttagaaataaaaaagggactccttcgggagtccctaacttatgctttatgctttcttacgcggattaccaaacgcagcgtcgtgagcaagaactttacgagcacgagatgctagctggtcagccaatggattgatacgagagttagacccacgcttgtagccagcgcgtttagaggtaccaacaacttcttttactgcagtttttgctttagcttgtttagccattttcaatcttccatattggagaaatacaatgaactggtcttattgacactgccagttctcaagccctcaaagggtacttaggtttttggatatttaacgacaggataaccataaacctcgtcaacattcaagaggcgcaccgtaaaacattttccggcagcttctcgtttttagctcacactaaggcagtgaatctccaacgagttacttcaaagtaactacaaaatgccgaactgctttacgagctgaactgcataagcctttagcgaattttaattcgtcttttaaaagaagagcatcacgaaaatcagcttgtgctggattaagatgtttaaaccctttcagtacttcaagagcttcagccaatacatcaattgatgcaccgtaattttcatgacctgcattccaggcagtgcgttgcagagaaagagcgtgttcaagttgtttgttcatttaaaaagcctcaaatgaggtgaataaaaggaaggcggtataaggagtttaagccctgcgccttccaccatgtaatcagaaagattacaataatatttatatcacttataaacgcacgctaacgatttataatgacaagtaacaaatttttgtttaatttctttaggttgcttaatgcctaacgcaactaatggatgagttacattgcgaatattaccaacaggcaacggagttaaatcaccaatttctacgaagccagcaggtacatcagcaccaattgaatatacttcacacatttcaggaagttggccctgaatttctttacctaagaaaatacccgaatctgaaattttttcatcaccagattgtgctggttctgataccaaaataacaaattcgcctagagctttaataggaagttccattcttacagtttaccttgcttgttaacatgaattaataataacatgaatttcttaaagcaaattactgagtaatcaactgaactaaatgttcagcctttacaccattaacagaaattagtttatcaaaactaaaggcgcgccagccttcactttctgtgtcataaactggaagcatatcaacagcttcttttcgtttaacttgagattctttgactaattcaaaatcactaggaagaatatctcggtcacgagtgcaacgcattgaacgaatggttccgtctgctttttcaaaaaccacctgagattcgccattactcaacacagcttttagagtttcacgaagttgtactttttgctcggttgtaagtttcattttattaacctatgatagttttaattttatgtgcgccacgaacctttaatgcttcatgaagttcttgacaacgtttaagccgctcttcaactttggctttatcatcgatagatacataatcagtttgataaagtactttccatttagaaaagaagtttttcttatattctactgagtatccaatattaattttagcgtaagaactcaatccagttgtttgtactaatcgagttttcattcttcaattccgcagtattcgttaatattatgccagttcaatttaccgaggcttttcttgggaacattgaacacctctataccagcagcccgtaagatattatcccagccaggttcatttttatcgtaagtctcacaataaactaatgttttaattccagattgagcaatagcttttgcacagtctggacatggagaaagagttacatacattgtagctccttcaattgatgaaccattacgagcggcgaacaaaatagcatttaattctgcgtgaatttcattcacttttgaccaagcgctatgagccgctctatgtttgctagataaaccaaccctcgttggcttccattcatcttgacgtaggcccgaccatttttctgtgattaaccagccttggtcatctgcatggtcgcaacagttaactcctcctgccggagaaccattatacccggtagaaataatgcggccatttttctcaataactgctcctactttccaagagcaacatttcgattcttgagaaactaagtaagcaatttggagcactgttgaagctttcattattttatcaccaaataagttgaacgatgtgttttaacaagacttatttcactaaacagtttttcaactgaaagtactgtcccgataattatataacatccgtctttaaagcgttctttcttatctaataacacatcgcctgcaaatcttttacctacttcaatttcttcagtaacccaaggaacatctccgagcagcacagcatcgtatgttgacgggtctattgaattaaagcaccggtcaaaatgcaaaagagtttttatttcttggacgccgtcttcagtaaattctttgtcttcagacaaccaaggacgacaaggacgatattcagtttcacgaattcttctaatagcttcaactagtgtaatcataaatttcctaaaatatttgtcatggtaactgaaccatcacgatttaactttaacccggtgtgttgagatttaaatccttgctcaactgaaataataacaccattagttaacgggtcatttcgcatagtaactgaacctccaggaattttgttagcaacctgctgcgcaaattttttagcaagttctgtaaattcagttcgactaaatgttttatctgatgtcattattcccacccatacactattttgaaactaataaaatcttcattagaactattaaaatcatcattatcgcatggataaacaatgaagtcaaacgtataagaatatgagtcgtatgaagttatgatactatactttatatctttccatagaacaccaaaaatcgacttgatatcagcatccatacgtaagtacagtgcattacttgggccttggttaatataattatccatcacgtaagaaataatttctttagcaatgccagtatatgacagactactatttgctcttactttcattagtgcaccgtatgtacgttgactttaactacgaattgagttacaacttctccaataggattaaactcaacatgatactcgcccttatagcgagaatttaaatctgttcgaatttgggcaagcttattgataagcgctggtgtcatactcatacctacgcattgctgtaaaagcttgtatgcttcttcttcaacttcatgatgtttgttatacattatattcttccaaatattctagcacaatttcaacggatttaaaaatcccgccaccaatgagttcttcattgtcatagtccattatgtctaaaccaatagttccatcgtttaaacaatatgcacaaaacagatattcttgtttcttttcaatttctgtaattaaatcaaaaatctctttaagattattcataagtcgtaggttttccatggtagagggattttagttttcttaacagttaactcaataccgggatttaattttctcaacctaagacattcatcccatgctttaatttcactggaaaatgaataaaacataccatctacagtaacaatacgtttttcattattcatattaacaatagcccagatttcatgatgagttgccattaaaaatctccaaagttaacttgccagcattcgacgccaatacgtcgccacatttctaccacttgagaccggtcatcaacggcaagaactacattatacttatcagcaatatgggtccaaaagatttcttctttaactacatcatctttgcgagaatcaccttgtgcccgctgaaaatggtcttctggaacacagatatgattatcccataaccaatgaagagtatgaatacgatactttaacggttcatctttagttccagattctctaccagatacagttatgattttatatcctttatctgaaagcattttaagaaaattaacaaccatttccttaggagtatcttcccggagtttttctaagtcaaaaggagaacgatgactgttatctgcaagtgttccgtcaaggtcaaagataactgcctttggtttagacaaatccggaacgtacattttctcaacttccatcaactgatagaaatgacgcagaatatcgattggaacagccttttcaccacgatactgattacgcttaagaagttcagtccatggtacgctaaatttcttgaactcaatcacatactgccccttgaaacgaagttcccacttttctgtggtttttgggttcaagttcgtgtctgcaataatgacacctttggtatgctccaaattaagtaaagactccgccgcggacatctgagctgtagtcacagctttttcgcgctgcttagagtatttgtaatcattccgcgcatttagaccaaacaatttctcacggaaatcatctcggcttagaatataccaacccgggttctttgatacaaactcatttgcccaggtgcttttacctgaaccggggcaaccaacagtcataataatctttttcatttttcacctaagaattgttcaagagcttcgattttattatccaaagggccgaagcccttattttgtaaaaagttttactgtataatacacggttacacaaataattaaaacaactaatgggtccatcatttacaccgctcttcatttaagtctttacgataataacatatcgtagcattagcatcttttacgtatctgcttacatcatttaaccaaattctcatttcttgagattcagcaaatggcattccaacccaggcttctccgtcgataactttaacttcccacttcccattccatactgatataggttcgggccatgatggatggattaatggtttagggacagcctctggcttgctagcacaccctattagtaaagccatagatactatcagtaacatgttccgaatcatctagttgattcctgtaaatctctggcaaatgaattaaaactctcgttgatttgtttttcgacgagtcttggctttgcagccaccacgttgcttttcttcgcgtctttacgcattttcgcattagaagtttccgtgattaatctatttgaactacgtttctcatctaaaatttttatatttttaaaatcttctttgagttggtcaatagatttggcctgctcatctgcaacttcttttactgcatgcaaatcttcttttaacccttctacccgctgtgaaaggtaaaaaatagaacctgcaacaacaattccagctattgccagcgtttttatacttatttgcataatttgacaattacctctacaatatcgtcttgagataatccgttccataagacgtgatttggagcgtctgattgtgacactttaaatccttgaatttcatctgccatttctgacgaattcatatacggattatgaatacctaagcgatgcttaccataaattgggtcaactgttatatagcatttacaatcattgataagcacgttaggttgagtgatattaataatcacttctgctccgtatttcgtaagctggttttcaagaaaatttttcatttcttcaacagcttcaggcatcgcttcttggcgagcctctgtaactttattgttgtactctttttgctttttgcgctttttctgatccgtggcgaatgcctttctgaggtcagttaagtatccaacccgccgggaacctttgaataccgagataccgtcgtgatgagtaccatacttttctactaaagcttcgttagtgataagttgtaagttcattgttaatctccgtttagttggtgtaagaacatagtatcataactaaacggagatgtaaacggtcttattcgtattcttttggaatgaacgttttgtagttcttcatgaacagctcgtttaccttcttgatagttgtgtcgtagtccatgctatcattgaacatcagcatcaaaattgtaaacaagaacggtaaacgttcagcatcagccagtctctttgcctcgcctgcgtatgttttacggtctaaaccacgaagtttttgatacgacgtagagataaacgacaatgaacggcgcagataatcaagataaacttcttcgaacttattaattttctttaaagaataagcatcgttagaaaaaagtcctttgaggtcatcagaagcgttattcacaataacttcaaatagtctatcattttttgtgatagagtccttagtatgatgtaaagctgtgtaccaatcagtctttaacttaaatcgaagaccattgtccatcacaaaaacataaccttcaatcccttccattgctttaatatcggatacaaaatctccttccggaatttcatacgactctaccaaatactgacgaagaacagggtctttagcgatttcgttgtatggaacataagctccagtatcattttcacggatattcaaaagaataaggtcttttttctgataagcgagaacaatacgattttcaggagatacgtattcaaagttagctgtaaaaccatcacgagctagttcaagaagtctccacaacaacgcttcatgagtgatgctagtaagcatagcagaagacaaataagcttgttcagatttaagcgaagactttgatttaaagcgaagaatattgccatccaaatagcttgacaccaaagaaccatcttctttcttcatcatatgaaccgcgttagaaagatcgaggttcattgtaaacggagtttcatttaaattaaagaatttttccattggtctagaagcaattcttactggtactccgtcaatcatttcaaacataattcctcgacactctaaagcatctggaagcaaccaatctgaatatgaagcatagttataactaaaaattctgtactttagcccagacggagaaatatcatcacgataaaagaaccgtgattcgtccgtatcattacataaattcatcaaattgtcgaatagctctttcatggtttagccttgtgttgagtgttccatggaggattaaatcttttaataaacataggttcttcaagagacatcgtctctaccgacattgtaccaagttcatttgtcatgcttaagttaaagcattgacgagcccagaattcaacttttttaccttccattaaagcttctaaaatctttgctgattttacggaatctgaagtctggtcttttctgtttatagcagtacgataataatttatacgcttacgaagattttttgtttttccaatataaacaagttcatcatcaacagcaattgcataaactacattcttcttattggggacctctatagttttaatagaggcatcttctagtaattcaagctcagcgtattttataaatgaaaattcattagcaatatctttcatattaaaaaaggggccgaagccctttaccttaaaaatattttttgaaaccaataattatattgtcgtcaacgtcgttgtcgatttgagctactaagtaacttgaaatttcaacttcctgcggagcagattgtaccgcatctgagtttaagtattcacgaatccatggaattggatgacgagtaggagcatcgactattgggcatggaagaccgcaactacgcatacgagaaactgtcaaatagtcaacaaaatcatgcagaatttttacattcagacctggcaacccgccatctttgaacaagtgaatagcccattctttttcttggcggttgatttccatgaagatatcaactgcttcttgttcgcattcacgagtaatctgaacccattcgtccccatctgcgccttgttgaagctgacgaataatatattgagtacccttaagatgcaactgttcatcacgagcgatgaacttcataatcttggagttaccttccattatttccatgttcttgtggaaattaaatgtaccgtacgttcgattgagctcgctagttctcaacccgttctcttatgaactgctgcatattactatgcagtccagactatatcacaatcccaaagggattctccccatttcgagtcgcttgaccctacgtccgaagactagtcgttgaaccttgctttgcaattgtcgccgtgccaacgtttatatgatgacggtgataaatccttcttatcacagtgtggacaattcaattgaattctatttggattgaatttacatcgttcgttgtgttttagataaaatcccggacctttacctttatgcccgcagaaatcacatacaatttctttttgggccggatgggttccgttattaaccatttctaatgtttttatcgatgttcttttcttatgttcttctgataagaaatgatgattgccatcatttactctttttgattgagtttttgattgcattttaccaccgcaaaacacatgaatgccttcctttatagctttggcattacggtctttaacttttttaatatgcccgacttgttgaaaattatgtatacccaatgcaactcgatttaaattagacttacgttgtatttcaccgcttaaaaggtgaaatgtaccgttttctattttagttttaatagaggcaatgtttttagctcgcacttcaggaatattaaaaacttgtatttttcgcgatttagctgcttttgaagcaagttctgaaatttcttctgcagaatatttcattcgtaactttatcgcatgacaagcttgataatctttttgagctaaatgtaaatcataatgttcctgtatagaaaggcacattaaattatctaaatcattattttcacggttcccgtctttatgatgaatttcataagttctaccattttcatcttttggtatagggccgttttcttttatccaaatttttctataattcaattgtgcatcctcttaaaagtataaccatatttataatattatactaattaaaggtgcaagcaaaaccttggctgctagttttcctataaaggactttccagcaattaaaggagttttcgataagcgttaccacttaaaggcgcattttacgcaaaagatacataaaaacgaattgcttcaagaccattgatgacatgcaaacacaaataaagtgctttcattaagtcacgtttagtttctttaacttggtcttttgcgtctcgaataagttcttcatcatgctcagtggcttcataaaactctaaatcagctttagaattttcccagtaacgagttttacgaataacatcatcatagtaacggccaattgattcagctcgagccataatagcatcatctaaaacaatttcatcaaaaattttggccgggtccgcgaacaggttacgcataatgtgcgtgtaagaacgagagtgaatcgtttcagagaaagtccaagtctggttccaagtatctaatgacgggtcagaaatcaaagcagaaagaactgcagcaggagcccgtccttgaatactgtctaaaagagactgatatttcaagttgttaataaaaatatttttttgatgctctgggagtttatcaaattgagctctgtcagtcatcaagttaacttcttctgggcgccagaaaaatgagagctgcttttcaatcagttcttcaaatgtacggtgacgctgaatatcataccgtgctaaaccaagaccgcttccgaaaaacatcggttcattcaaaacattaactggttgtgtattaaaaactgtactcatttattcacctttgctcttcctcttatccatccttcaggaatagtttcagaggattcaattaatttattcttaaacccgtcgtttatacaaattttaccacgggctcctttataatttcgtggttttattttatcaaaattaggagtaagcgaatacgaccatctgaatccgtaagcatactggaaatggccttcagcgcaatatttgatattagaaccgttcccattaactgatttagcagcttcggatatagaaggaaatgtctctatgaaattaccgtctaaatcaaatttgtgtatagttacagaaacttcttctgcccttttaattaatggtaatttaatcttttctaaagaatctttagagtgctttttgctaagcattcccttggggtgtgggttattagctaaatattctttcatatatttagaggattcttcggaaaatctctctcggtgccaagcatataatctattattaattctatttccggatttatcggtgcacattaaagtgatagcacataataaaccgtatgttttatgaattttataaagcaactgatgcgcaataaagtgttctcttgcgctcaatgccactaagttatctttatcatctgaaccacccatgcatcggggaatgatgtggtgattttcggtatagccgatgaaaggagaggcttttcgcctctccattaagctatcatatattctttgatagttcattataatttacaagctgcacagtcatctgcttttggttgctcaacttcatagtcatctgtgccagaaccatctctagtgttatgataatagagactttttccgccaaaataccagaagtaaagcaggtcatcaagcatcacactcattggtactttacctttttcataattctgagggtcatagtaagtattagcagaagcgctttgacacaagaacttggtcatgatagcaacttgagttaggtaaggtttgttaccttgtttagccaatttccatgcataatcataaaggtctatgttgtgctcaatattgggcacgacttgattaaaggaaccctcttttgactctttaacagagactggtccacgcggaggttcgatgccgtttgtactgttagaaacttgggaagatgactcacacggcataagtgcagataaggtgctattacggatgccgtggctaaccaggtcttcccgcaagctctcccagtcacacacataattgggcgctgcgatttggtcaatctttttattgtaccagtcgataggtaattcgcctctagaccaacgagtttctgaataatattcgcaaggtcctttttctttggcgagtttaattgaggctttaatgagtgcatattgtaatctctcaaataattcatgagttaaatcatttgcatctgcatatgatgcaaaattatcagccaaccaagcagcgtagtttgtgacacctacacctaatgcccggcgcttcttagctttcaatgcttcttttacaggataaccttgatagtccaataaattatctaaagcacgaacttgaacttctgcaagttcttcaatcttgtcttggtcctgccagtcaaaattacccaacacaaatgcagctaatgtgcacaacgcaatttcagggtcatcactaccaacgtctttggtcggtaaagcaatttctaaacaaagattgctctgtttaattggcgatgtttcacgaatgaacggagtataacggttcatattatcagtgaacgctggatacactcgagccgtgccagaacgttctgtcatgaacaattcaaacaattcaagagctttgatgcgtttcttgcgaataattggatttttttctagttcttcatacaattcacggaatttatcttcatcccggaaataagaatcgaacaattcgccattagccacatctgggctgaacagtgtgatgtacccatttttaccgagacgttccatcatcaaatcattgatttgcagaccgtaatctagatgacgaatgcggttttcatctacacctttgttgttctttaataccaacaggttttcaacctcaagatgccacattggataataaagagtagctgcaccaccacgtacaccgccttgagaacaagattttactgcagtctgaaaatgtttccagaaaggaattacgccagtatgttttacttcgccattaccaatttttgagccttcagctcgaatcataccggcgttgatgccgataccagctcgcttgctgatatacttaataatactgttacttgccgcattaatagaattcagtgaatcgccagcttcaataactacacaagaactaaactgacgagtaggagtacgagcccctgccataatcggtgtaggcaatgaaatctggcgagtagatacagcatcataaaaacgaataatatgagccagacgattttctggttcttcttggtgtagtgccataccaattagcatgaatgcaaattgaggtgtctcgtaaattttaccagttgaacggtctttaaccagatatttttctttgagctgcatcgccccagcataagttaaatcaaaatcacgttcgtgtttgatttttgattccaagaaagtgatttcttcagctgaatacttggagagtaattctgaatcatatttaccctcattaacacaataagaaatgtggtcgataaaagacggcggttcgaattgtccgtacacttctttacgtaatgcgaacatagccaggttagaagcaacatattgataatccggttcttcttttgaaatggagtttgcagctactttaatagcagcttgttgaatatccttggtgctaattccgtcttggatatggcttttaatacgctcatataattcatatgggtcaattgaagtaccttcagcggcccatactaaaatcttttgaagtttggatggaataaattcctgtttaattcccgatgattttactacttgcattttatatccttagttatcagataatttattttatcatcaaagacttttagcttatcgtttcctcaaagactaaaatggcagcccgaaggctgcctgtagtgttatatttattatttaagcttcttttactacaacaactttaaccgggcttcggttcatgtcaatactcagaaatattgattggataatataaaaatatccattaatcattatttggccgccttctttcaattcacgaccaatatcactttcagtttcggcaaatactttatcaccgtgctctgatgtaaatttaataagcataatattcctatagaaacagattcaacgaaataattataataagaaaaatgataagcgcaatagttagtttgttcatagcagtcatactgccagtatcatttacaaatctagtaatcattcacaagccatcagtgctgcttcgagcacttcaattaagtttcgaacttggtctagaggtaactcaaaacctaagccagaatagttgattttcagacaaactctatcacagtaattcgggtaaactacagcccgaatttcttcttgatatttgtctgtcacatggtctatgataacctgatgaaacatatcaccaggttcattcatatctttacgtttaatcatacagccatttttcctttgattgtagggtggctctcgtaaccgtcaagaacaaaatccgccggggtcatatgagtgcaaacccaatacaactgcatttcggtgtcccagtcttcaaaaccttcaggaaaattaattttcagttcacagagttcttttggctttcgacggagaacttctttacactgttcaacgtggttcaaatatatgtgagtattaccaccagtgaacaccagatgtccaggttttaacccagtcattttagctacaatatgaatcaaagcagcataagatgcaatgttaaatggtaaaccaaggaagacatcaacgcttcgttgggaccacattaaatctaaaaacccattacgcacgttaaattggaacgcatagtgacacggtggaagcgccatcttagagagctctagagggttccaggctgtcacaatctgcctccggtctgccggcatttcttttattctatcaatgactaacttcaattggtccacgcccatgaaatcacgccattgtttaccataaaccggtcctagttcaccgtcagtataacccatcgaaacagcttgattttcataattatcatcccagacggttttcccttctgttcgagaaccatgttgaatttcacggaggtcatctacatttgttgagccagataagaaccacaacaattcagcaatgcacgctttccacgcgagtttctttgttgttacagctggaaatccttcctgtaaatcaaaacgaagctgagtgccaaatacagcaattgttcccgtgcccgtacggtcatcagtctcatagccttcatcaaaaatgtgttgaattagttcttgatattgtttcattgttttccaactttaaagagaatttcggcgcctttcttttcaaaacggccttgcatccaaaggagaccattttcaactttcatataaccaagattaatatagtccaacatctgacgattactgaaaaacgctttaaatgttccatggttagaacgtacatatataccattagtgcacgcagagtggatattctccaacggcgccctaacctcataaaacccatgaggaatagtaacatatttctctggatttcgtttaaactggtcaattgtaggggccattccatcgataaccaaagcccagttcttttgcttctcttcaacaattaaatctaatttagcagtcaaaatgtttcctctgttaatgggtcgaaaaatattacattaccggattttctaaaccgtccaaccattgaacagcaaattccattaaagcgagtttgccctacgcttactgattgaaatatagttttcatttcacgacacaattcaagacgaccaataaccccatcaagcaaacaatgagaaccattccatgaagaaattgagtcaatatcaaattcaagaagataaattccatgaggaatgttgaaacgctgccatttcttttgcacttcatctttttccaataaagtgattttgtatattttattcatcacgccctctcataaacatcttctgaaatattagtaagttcgtcaatattataccaatgagtctcaacaagttccatatcttcactgatattccatataaaatctgccgagagtttaatatcagaatttacacgatgttttttgcgaatagttgtgcatataattttatctgcgtaaattctcgaatgctcaataatatgtgcaccgccaataacagaataattggcatcatcgcgttgaataaaacaattatacccaaaatcatgcccagaaacttgaactttattaccttctaaaaattgttgaaactgtgtttcattgatatagagctcagcggtttgtcctgatttagtttttgcataaggacgagaaaaatcttgtactacaatatgagaacgtccatgaagttttcctcttaagctcataaatgtttttgcacccataataagaacagtatctttagttctggaagcaaaattttgcatgtcttgcttgatatgtccccatggaagaccagtgtttaacccaaacgcaaattcatttttattgtcgacagttttagtagggcaatacgcaaaaattaactgaatcattataatacctcaacataagcgatgtcagatggctcgatataattggttttgcattgacctttataattattaatgcaaaaataaaactgcgatttggcattgctccaaaccaaattgaccttatgcaaatcaccttttatagtttcaactaaaaacacgctatcaaaatatgctttatttgagcaaatatcttcaatatttaaatcttgaaaaatttccattaaatttatagttttcatcatccttgcctcattttacaattaggacaaattccacctgcgaacaaaagtccgccgccgcaatatttacactcccagtcggttaaaattttcatcttaagttcctaatacgttcacggatgatattcaaaaactgccgtgtcatttcaatgttagcttctcgccataagaagttgctattcatagcattatattcagcagtcacatggtcgtttaactcacataacgcgtgtagtttgccatgttttactgcatcagaaattgctttatcaaactcttcttttgacattgtaatttgcattatttgtttgaaccctttttagaggctaaaaccatggtacgaagcattctaaggcctttatcaacagcttcagcaatttcgcccgccacgttctcattgaatagaccgagagttcctgatttgatatcttccattaaaatgtagtttatgtcattttcagttaaagaaatttctgggccaattccacctgaaaaattaatcttacagaatacattctgtccacgttgttcttggtagcgaatattaatgtgtgtcacaccaggaacatggtcaaactgacaacgaacattaacttcaggagcagcagaacaccagatatcagccattataattcttccttagcttctttaaatgattttttgaaatcttcaacgtattcgataggaatccagaacccagaaccataaccgtcttcatagcatgggcacatatcgcaaaactgttgccaatgaatagttttaccttctacagtgaagggagcttccatttcgcataaagactcttcaacaagttcaatcatcgctggatttttgtcataaatgcagaaattccaaacacctttaactgtggaatctttaatgctttcacgttgtaatttcattttattatcctcatttgttttgatagggttatagtatcataaccataacccgttgtaaacggtcatttgaaagttttttgtaaaagttcaatgatttcgtctacgttttgttttgtaacaacacaatgaattttagttacaccgtcagtatctttacattcgtcatcaagaactggttcttcatattcgcgaaagcaatagaactcattatcgtaaagttcaaaataacaatcacctagaccatcgtcgcaaaagcactcaccattagcgcagacaatttcagttacaaaatattggttgtcaaataattctatagattttacttcaaaccaaccgccattttcgtggataatatctaccatttcagcattattcaaagggcttaagccaataaaatcattaataagctctgggactagttcgtattttttgcctattttcattttgatttcctcaattattagttattgaatgttaaaagttgctttaatcaatgcaatggcttcagcggcgttgctttgattaacctgcatgtgaatggtttgggtgtcgaccacaggagaatcatctacttccaagaagtatttgaattcatattcagcgagttcaaaatagtcttcacctttctcgcacatggaagtaaggatggacccgtctttcatttcaaccttttcaacaaagcactcaccaccatacatttgcatgtcaagaactttaaaactatctccgcactctgccatccgcttaaccataagcgcgttatcatctgggcagtcagcgatgaaacgttttttagccgccgggtttaatacgtaaattttaccgattttcattttgatttcctcagttattctttaattaaacccatataaaacattttatcttcgggttccataccttcgcagtactcaatttccataaaacgattaatagcttttaaaggaccattaatgacaacatttatgttccatgatttggaattctgtgaattagcgatacgaagttctgggtatcgattacgaataacttcttcaatgtattcaaaatctacaacatcaatatcaacactagccattttattttcctcatttgttttgatagggttatagtatcataaccataacccattgtaaacaattaattgatatcactcatatacatttcgatatcttcatattcgcaaatttccaaatactcgcggatatcttcaacttcaccaatgatttcaactaaaggccatccgccagcgggtccacgttctgctataacgttaaactccaatttaccaggaagaatttccatgaaccgagtgcaatcatcaaattctaaatcaataatcattccgccttcatctttgataatttcacatactaagtcaagttctaaacgtgccatgatattttccttcatttgttttgataaggtaatagtaacgcagtttaaagctgatgtaaacagttaattgcaaatttctgcatcaactttactctttcacaacttaattcattgcctagaaaaatcatagacaatagtcatttgcactctcaataacatcgtatgaggttataccataactataacctcataatgtagtatacgtcttattgttccataaattcatctagcaaccaatcaatgtcttcccattgattatcttcaagccatttgcggatatttttttcagacccgcgaagataaatcactggccatccgccgccggtgccttcttcgattataaggtcaaaatcaagaccatggtcaacatcataaaaacggcttccatcctcaaaaattaaatcagttgcaattccatgctcattgcggtgaacttcagctacaatatcaagttctaaacgtgccattttcaatttcctcatttgttttgatgaaataatagtatcacagtttttggaggatgtaaacggttagctccagaaacaacaaaggagcccgtaggctccttttattattaaagaccgttcaaaatgtcgtccaggtcatcatcgtcagatgaactaacatctgaagtagaattgttagttgtagaaccagatgagaaagcttccatatctttatcaaaagcgtccaggtcatcagctactttatcagcctgtcgttcagcttttgctgcggcaccgcccatagcagcagttcccataactttctcaaatttcttttggttgtcgtcaaaagatttaaatttatctttagaggtcatttcagttaaatcaaccatctggtcattgagagatttctgaacagattcatcgtcaatatttgggatttcagattggcccaagaatttcgattcgtcgtagttactaaaaccggatactttcttaactttcagtacaaagtttgcaccttcataaggacaagttacatcaactggagtttcacccatatcagtatcaactgcaatcattgcgttgattttgtcccaaattttcttaccaaaacgatatttgaaaactttaccttcattttccggagaagccgggtctttaatgaccaggatatttgcccagtaagaagttttgcgtttcagtagtttgtattcttcgttatttgtgttgtagctatcattcttggaaagatattgacaaaccggacaagaatcgtaatcaccatgagtagatgtgcagttttcaatgtaccactgattatttttcttaaagccgtggttaatcagaagagcaaacggagcagcttcttcatttttggaaggcagaaaacgaatcaccgcttgaccattacctgcgttgtcgagcttaagtttccattcacctttatctgaatcttggaaaccacctttgttaccagacagagcagccagttgagcttgaagttgagcgggatttttacgtttaaacatattatttaccttattaatatttacagttttttacagtttttacgaacagttgttttgcttctaatgcatcgacatcaagaattttcttgtatgcgttgagcttagttgaataatttgaccaaactaaattgtccgttgcctggtcatgattatttataatatccaaaaatgaatcaagaattataaaagtttcgaaagaaataatactactttgaagtagtttaaacacatatgatgtgttgattttgttattatatacaaatatttcgtttagcgttttaacttcaacttttttgctgaagtaatagatatttctgacgtcttcttcaaaggtgcttttaatcattttcaatttgccgatatattctctataaaatacaagagcatcggcatcactgatatctccaatccaagcatcttgatttgcaacaagattgctcatgaagattattgcaagctctttaagcttgtacttttctgatagtttttcaaagaaatatttatcgcggcgcttctgatacgccttatcagaaacccgcatacgccagttgtattttattacatcatatctgccagcgaaatgattttttaacatcaagtacaacaaatatactgatttgccattgatgtatctattaccatcaggcggcatgcggattttaatcataacagaaattctaacgtattagttttttcaccacgagagacactaggtcttagcatatgctcatcgatagcttctgacctaattttttcaataataccggatggtataaatttagagtacattgtttctgggatggaattttcttccatccacatagtagtagcttctaaataccctaatccgttttcttcaactaaagcttcaataataaatccgttttgttgcttatctagcaactcatttaatttatcatttttttctttagtagatggaaccggaattgtttcgtcatttagtgaaaatagagtcataaatttcaaccactttatctttttcttcttcaaactgttcacgagtatctttatgatacattgccaataattgattaaaagctttcgaatcgacgcctaaatcatctttggcagctgctttcaaatcagcgattagtactcgataaccagaaattttcagaagatggtcagaagcttctttaattttcttacggaggtcttcaccatgaatagcttcatcaaattctacagcaactttcttttctttggccattttaaaactcattaatgtgattagttaatttagcaagacccgacttcacaaagtaagaataaatcttaccgcgaggaggtgttttgtataattcataatattctataatcggtttagcaatttcttctggaatataactaaaatcgataagaactctattttcaagataacgattatattcggcttctgttaaaagcgatttcattccttctggaccagcatctaaacactgttcaaggagcttagttgaaaacgaaggagttcgctcaccgtcaattttagtgtaccaataatcagaacgtactttaactgatgcgatgttatctttgcggtcgccttttaacactttagttaaacattcaagttcagcgtcaccggttttgcatacaacaaatttcttttgcatcggggaccactgtttaacattaggatatcgatgaagttgtttaaagtcaccgtcagaagaaacaatcataattttatgtcccttcaaggaaagataaggaacaagaataccaatatggtcatcagcctcgtatttgtcaatattcatgacggtataaggcatatatttctggaattcatcaataattgtgcgaattccagtaaaatatccttcccaatcccaagtgctttcttcacgaccttgtttacggttctttttataataatatgcaatatcacgacgccagtaacctgatttagcgttatctgcgcaaatgataactttttcatatccttgtttgcggaattgtaggacattctttttgagtgtacttaacacaagatgtcgcatcatagatggctgaattttctctttatcagagaatgtgtttaacgctgctgctaaagcaatttgtgataaatccactaaaacaataccttcttttgtttggtcttcttcgccaaacagacaatctagatttgacattgaataatctcattcagtagttaacgtaactattataaatacattctataatagcaaataggagcaatcatggccgaattaaaacgtaagttcagagctcaagaaggtctggacgctgcgggtgagaaagtcatcaacgttgccaaggcggatcgcacggtcatgtccgatggcgttaacgttgaataccttatccaagaaaacacattacaacaatatgatagtactcgcggttatccagcacattttgctgtcatttatgataatcgtatttgggtgtctaatagagaaattgctgaaccagcaggcgattttactgagctttactggaattctgtacgcactgatgcaaaatggaaaacagtgtcatctggaacaacaaatttaaaatcaggtgattttatttcagctgacactgccggaagaacagatatgaaattcattcttcctagcaatccgcaagatggcgataccatttttattaaagatattggtggtcagccaggatatgcttctcttactgtcgatgcatctattcaatccatagtatggatgggttctcaaataagaaccgctcaaatgacgcatccatactcacaaatggttttcgttttcagtaatcgtttgtggcaactttatataagtgataacgaatcaagagccacttatataactccagctagtattcatgaagcgcaagcaggcgaaaatctagttagaagatttacttctggagcagaagtttttattactcttcctaaacatgctaacaacggtgatataatttccgtagttgatttagattctcttaacccgttgtttcatactacattaaaaacttatgaccaaaatactagtataggccaagttggaactcatgagatgcagttccgtacttccggagatggatttgtagtatttgattcagctgataatttatggagagtatgggacggtgatttacgtacccgcttgcgtattgtaactgaagatactgacgttagaccaaattctcatatcatggtttttggggctaacaatgacgaaataaagacagttaatctaaacttgcccgaatctccagctattggagacacagtaaaaatttctcttaactatatgagaaaagggcaaacagttaatataaacgctactggaactgacacaatagcttcaaatattgaacttttgcaatttccaaaacgctctgattatccgcctggagctacttgggttcaaagcagtactttaacgttcaatggtgatgaatcttatgttccaattttagatttatcttacatagaagatgaaaactctcaatattgggtagttgcagataatactccgaccgttgaaagagtcgattcgactaatgatgagacccgagctcgtttaggtgttattgctttggcaacccaagagcaagctaataaagataaagaagataatccagaaaaagaattagcgattactccagaaactttggctaaccgtattgctactaaaatccgccgcggtattgctcgtttagctactcaagctgaattagaagttaaaactggcggggctttattagatgatgtgattgttactccgaaagttctgaatgaccgtaccgctttagaagaccgtcaaggcctcgctgaattagcaactcaatcagaaactaacgatggtgttgatgattctagaattgtaactccgaaaaagttacataaccgcaaagcttcagaaatattaaccggtattttggcgttagttaaaactggaatttcaactcttgcaggtgtagatagagatacaaaaggaagtaacgtatatgactatactgataatgaaaaagctgtaactccagcttctttatttgaaaacaaagctacatatacatcacaaggtggttcatacttagctacagaaaccgaagttattcagggcactccacatgaccctaaagtgcctacagtagtaactccagtcgaattgcataagaaaactgctactgaaacacgtattggttttagtgaaattgctacgcaaaacgaagtcaatactgggacagatgatttccgttatgttactcctaagaaattaaatggccgtaatgctacggaagatttaactggtatttcgcgagtcgcgacagacgctgaattcgctgctggggagctagataatgttatttctactcctaaaaagattaagaattacttttcttctcctgaccgtaaatctgttgttactgaatctggattagttgaatcaggtaactcctgggaccattataaccttgatattcaaaaagcatctgagacgcaacgcggcacattacaattagccacacaagttctcactgacgctggggttgatgacacgactgctgtaactccgaaaaagttacaagctaaaaagacatctgaaacttctgaaggcattattcagatagcgactcagtcagaaacaacaagtggtactgtaggaaataaagctgttcctccgaagcatctaaaatatgctatccaagaacaacctgattgggaagcttctcctcttcgtagaggtccagttaaattaacagaaggcgctttaacgtttgttggtgataaagaatttggttctggtgtcaaatttgacactacaactggtctttatattaatgataatgataagttaaatgccggaaattatttcaaatctggatatgctatatctccctttgaaatgaacaagactcttcaaaactttttgccaataaacgcaactgctgttaattcgcataagttagataatcttgattcaactcagttcatcagaagagatattgaccaaacggtcgaaggttcgttaacgttaacgcaacaaacgaacactagtgctcctctagtatcctctagtactgcgaagtttgttagtatgttggttactacagaagctactatcggggacgctacgggtcactctgtgattaatttggacgctaaaaccaataaatgggttattgatggacaagctaattctcagtatttagattttactgccggaactacagatgttcttaagctcaaacgagatggagacgttaacgtagcccaaacattatcggctggtaatagagtagatgcttcaaaaggctttagcgtcgaaggtggtataatggttattaatcctaccgccaacaatattcaaattggttcacaatctaaagctactaatatccaaacaacagacgccggaaacttgaaagttactgatacatcaggctcttctgtgatattgaccactaaaaacgctgttactatcgtaggtaataacttcgttaataaagctggcgattcaatgtctggccgtttagatatttctgctgcaatgagttcagtaattactgaagctaaagcgataggtcctcttactaacgaaactgtgggtaattggtcagcagaaatcataactgaagatatctacaaaacattgcctggatttatggttccaatttattctgacgatggcggtggtaaagtaattataggttatgtagattatgacccggctgatgcatcaaaacgcggtgtaagagctccgggtgtattatctcagattggaacgagtaaaaaggaattcacatatcaaatatggaaccctcgtccagctacagcttatacagatgctaaatcttctctttggattagaacctatgacccagttaaaggtgcattcaatgaatggggaagagtttacactactgaagctcctgttacgtctgctgaaattggtgccgtttctacagctggttcttcatttaataacttaacaatcagagactggttacagattggtaatgttcgcattacgcctaatcctgatacccaatctgttgattttacttggataccttaatggaaaaaatgatggcgagttttggtaacggatataccaaaactcaagttatttctgaaaataattccatcaaatacaaaatttcatttgctgcggggtcggttttttcgaccccttcttctgcttattttacctttcaggataatccaataggtaaccagcaagatggtgctggtataaatatcagagtttttaatccagcattgaatactgtatctgctaagaaaacatttttattgacacccaatgataatgacccaggcaatagagcattcattgaatatttgtcaacatttactcaagataatactaacttgttaatattcaccacatcaggtgatattaaaacgagcaatttagtagagaataaatttaagtccatttattcaactatgtggccaaataaatggatgacatctcggtattcatgtacttattgcggtttattttcaataaaaaataataaaattattgccgaaaacgtaacatattctgatggagttcttcgggacgaagacatcagacctgctttagaattcgtttatgataaagctgatgatattggagctaccgggttttcttatagagctgttgaagattttgaagaatacactagttccgcagcaacaataaaaagatatccagttgattctgcttcgggcgtagaaattagctcaattggaatatctcctggcgatattctgttctggtcatttgaatttttgcatggagataatattccgccggaagttccagggacaaataataataaaataagaatagaaataagatggttaaattcatctggagggtggattaaatcagttaatgttgactctaaccacgcaaatgctggaaaatggatacagcacgaacaaactgttgaagtccctgctgacgctgcccggatagttattcttgcttctaaaactactccaactgatacggtcggtactggaggtgttcgtagtatgattttaactgaaacttcgcgagctactgaggcattaacttctccatctgcgatatcagttaatggtattcgtttgaatactatagtttcaggagataacccgacgctgcttattttacctgctaacgaagttgattcaactggcaaaccattaccaggtgaagatgtttcaggaataatttacagttctgattggagagagtttgagaaaaatatttaagggccgaaaggccctttttctttgtaaaataacaaataaatatgaatatacttaagaggacttcatatggccgatttaaaagcaaatagtacagtaggcggagctccgatatggcataaaggaaattttcctttgtctccggtaggtgatacgctactttataagactttcaaagtctatacagaattcgataaacctcaagcagtagataacgattttgtttctaaagccgctggaggagaatatttaaaaaacgttaattttaaagaaggcttatcatttagcgacaaagatggagcttctgtttttattggtgttcctaaaaatactacagcaacagctacttataccgcatcaataaaattaaccggacaattcgctttagaaactcctgataataaaccatttatcatatttgacccaaacgagtcatttagccctgatgtatatcgcttaactgttatgggtgatatgctttctcgtcaaatttatgatgaatccggaagagttttttctcctgggaatactccgtcaaaagcgcaagttgggttagatttagtcgataacgctaagcaagttcaacttgaaaaatctggtgttcaaacaatgaccggggttttggctgctccaaactttataagtaccaatccggcaacggcagataatcatgtagctcgttttgaccagattgtaataaaagactcaattcaagattttggatattatagttaagaggacatatggctactataaaacaaatacaatttaaaagaactaaagtagcaggctctcgtcctactgccgctcaactcgctgaaggcgaacttgctattaacttaaaagaccgcactattttcacgaaagacgatttgaaccaaatcattgatttaggttttgcaaaaggcggtgaagtatcaggagatatcacgcagattggtaattatacccaaactgggaactataatttaaccggcgatgctactatatcaggcaaaactactacaagcacactggatgttgggtctgtttcagatctaagacagacaaactttagaccagtattaagcacaacaactggttctaattttattatatcaaactctggcggcttgattaaaccaattactttgacggtagaaggcacagctacaagttcaaacactattctgcgccattcagttgatactactgtagcagcttctggatttatcgattcaattaatgtttctttaaatccgacggacggcgctcttgttacagctctcaatggtactgtgaatatcggtagttcccttaaaactcctaaactttcagtttctggcgcggaaactgctttgggagattatagtatttcaatcggtgataatgatacaggatttaaatggaattctgatggtgtattcagcttagttactgacagtaattcaatttatacatactctcgtgataggacatattctaaccgtccaacaaatttccggtatacgtctgactttgatgctacgacacctgctttagctccgccaggcacatggttagcttcagttgaaactgcaatagacggtaacgcttacggcgatggaatgagctatctcggttataaagataccgcaggttatagtttttatttccgcggcggcggcacttttaacgtagcttctaaaggtggatttaatgtagacacagctgcggcttttgccaaaacggttgatgtatctgatattctaacatgcagctctattattaaagctaaaggcccaggtcaagttgatgttactagtgctggtaatatcgcacttggtggaactattcaatgggtgccttcgtatatgagcggaagcccaaatagagcacgagatacaatagctacagcagcttggggcgatgccgaccaacgaattaacgtattagaaacatctgacccgcatggttggtggtattatatacagcgcgcaggttctggaagttcttctcctacgggaatagaagggcgagtcaatggctcttggcaggcatctgatttaatttctgataatactctaagagtagctggagctttcacttgtactagaagaaactccgcaggatggggtgataacgctggatggtatgctggagcaacaccagtagtagctaaccaaggaaacgttcaagaaatggaccccggtgtaggcggtttttatcctggatttgctcaatataactataatggcactggatggaaccaagcatttgttctggggttactaggccaaggtgttcagagatggcgtcgtggtgtattagctcttagaggcgatggccctgttgatgctggacaacaaattgctcgttggtattttagtcaagaagacggaagtttggaatcagaaggtccgcttaaagctcctagtgttcaagctggacaaataacatcttttggtgtaaacgttacaaacgcgttaggcagtgcgtctatagctattggcgataacgataccggattgcgctggggcggcgatggtatcgttcaaattgtagctaataacgctatcgtaggcggatggaattcaacagatatttttactgaagctggtaaacatataacatccaatggaaatttaaatcaatggggcggaggcgcaatttattgtagagaccttaatgttagttccgacagaagaattaagaaagacataaaagcatttgaaaatcccgtagatattttaagcactataggcggttatacttatcttattgaaaaaggatttaatgaagatggaagtcaggcttacgaagagtccgctggattaattgctcaagaagtagaagctgttcttcctcgtttagttaaaatatccaatgatggaacaaaagatgttaaaagacttaattataatggtataacagctttaaatactgctgctataaatgtacatactaaagaaattaatgagctcaaaaagcaattaaaagagcttaaagacattgttaagtttttaactaaataagagcctacgggctctttaggagatattatggcagttcaaggaccttgggtaggttcgtcttatgtcgctgaaacaggtcaaaactgggcctcattagcggcgaatgaattaagagtaacggagaggcccttttggatttcctcatttatagggcgctctaaagaagaaatttgggaatggactggagaaaatcacagtttcaacaaagattggttaataggcgaacttcgcaatagaggtggtactcctgtagtaattaatataagagctcatcaagtgtcttatactccaggcgcacctttatttgaatttcccggagaccttccaaatgcatatattacacttaacatttatgcagatatatatggaagaggtggtactggtggagttgcttatttaggcggaaaccccggcggagactgtattcataattggattggaaacagacttagaataaacaaccaaggctggatttgtggtggcggaggcggcggcggtggctttcgtgttggacatactgaagctggtggtggtggcggacgacctttgggagcgggcggagtttcaagcttaaatctaaacggagacaatgctactttgggtgctcccgggcgaggatatcaacttggaaacgattatgcaggtaacggcggcgatgtcggtaatcctggttcagctagttcagccgaaatgggcggtggagcggctggacgagctgttgtaggaacctcacctcaatggataaatgttggtaatattgctggttcttggttataaatatctctaaggagataatatggcttcaaaaatttcgttaccaataacggatataatttttggagtgtgggacagagtttttaaagataatgcatccgggagggttttagtctcccgggttgttgttgtaattattttctttgtacttggattagtttggtcaaaaagcgatgcaatactaaccacttacagagattcgtcttacgatgcgtacgcgaaaattatacaacaagaacgcgaatcacgctttgaaacaactgctctggaacagcttcaaatagttcacatatccagcggagcagatttcactgctgtgtattcgtttagacctaaaaatttaaattattttgttgatttaatagcgtatgagggtagattaccttcaaccgtttcggaaaaaagtcttgacggtttcccggttgataaaactactaatgaatattctacacatttaggaggaagagtttttaaatcatcacaagaatttgcttttctaccgtcaaagaaaaaaacaactgaattaaaatacatgttcagttgtccttacttcaatttagataatatatatgccggaactgtttctatgtactggtacgacggagctccaagtgtaagcccggaacgacttgaatcgatatgtggccaagccgctcgtacattaggaaggtcacgttagaaattacttattggtatgcatcgtcagatgacgatagatatcttcaaatccttcgttgaattcttcaacaagcattcctttctcttctgcagttaaatcagaaattatttttctaaagctactctgatttaactgcttccaatttttatcagcactcatcgtttcattgataaaagcaataaagttatctcgtttatcaacaatatcatcgcgaccaaatttaattaacaaagacgcaatagtaacaacttcacgagcaacttcaaacttagtcatcttatatcctttagtatttaatttttaaatcaataaacggtatagaagtatagtatcaaaattttattggtattgtaaacggttgagtgaactaatttaaaacttcccaacgaaccatcagatgctcaggttgagttatgtactcatttccagaattgttcactcgaatggcgtagcggcctccttgcattgcagctgatatataccattcggctcctactggacgaaccctacgtcccatccaatgacagatttccatggcctcgtgaaaagagtaatcttctttgtccattgccaaattaatcatcatgctttcaatgtaacgctgaatttctacagtttggttgctcataattttcccttcaattctttagataaaatattatcaaaataaatcacggcagcttcaagaatgtttggattctgataagtagaaaatttgaaattgcatccagaataagaccctcccatgtacttcacatgaacaccgtactttttatagaccttttcttctgccctctcaatttcgtcaggaaaatcacttccaaactttccaaatttaattacttcagaagtgtgtaaataactgtgctctgtgacaatatttttatgcttgaaagtcatttgactgtcctttgtgtttttgtttgcgtttggagtcattaatggatttctttttatttttatgaaatccacctttgttaaaatcattttttgccactgggttattcaactaattttcctcttgcgtttttaagctgcttaacttgatgagccaaatgctgcagtggaatattttcatcttggttaagatttcgtctaattaattcaacaaaccactcatattttggtctttttgataattcagccctaagtttagcattttcttgttcagctatggtagctctctgcctataatccatttaaaattcctttgataattatttgaaccacattttaattctcatccatagggatttctgaacagctttgtttaccccgtaagaacgaattactggctgactttcttgaagttctttataatcagctttataaacttcgcaagcagcgggaacaaatgaactaattgctgccataaaatttgtacgttcacctaaaggatttactgaactatcgcgaacaatcaaatattttccagctttaaccttcacgatagttccaagatatgcaccatgataccaaatatcccaaccttctgaagtaggttctacacaacgctgaagttcattcataattgcaaaacggttcataatacttcctagactttaacaacgtgttcaataaataacccatgtttattttcgccttgagtgccttgtataaagattctcactgcgtttggaatatctctagcaaatatttcaacccaatatgtaccaccaccaatctcgtaagtgattttatatttgttcataattcctcacagtgtgtcaacagagttgatgatagtttcaatattttcttgagtacgaacgatatctaaataaacattaccatttttagaatgtttggcaatcataccaaggtcttcaaagtgcttaacttgctcttcagtcattttataacccgaaatacggaagttacctttattgctaacctcgaaactgcgaataccaaatgtgcgtttttcaaggaccgcgataaagttactacgataaacatcaagttttttgattttgaaaagttcttctttagttccaagaagctccatcataaaatctttatctgcttccatatcacccgtaatcgggcgagctttacgagtattgcgtttttctagaagttcaggagcattttcctgtgcataaagtaccgccgcgtttgagatgatgtcctgtgcttcacccgtgattactagaccatcaccagatttttcaattaatcctttcttaatcaaaacaccaatattactgttgactgaagatgcagtcattttatcttctaactgttcacgaagttcagctgaagtgatgaaatcttgttttgcaactttaactaaaatcgatgcagttttttcattcagaacatcattagaagctttgatgatataagttacttttgacattttgtttctccgataatctatttgtttgttgataaagtaatagtaacacagtttaaagcggatgtaaacggttgaagtaaaaatttttaaagttaaccagaacggttgagagtaacgcagaagaaaggatgattaattatatggaccgggctctagaatttactagagccgcatagagttacagcaaaatggagagaagtgcaagaagaagggctaaaccgcctaaaactacgacgattgacaacagcccggctactaagaccgggcttgcataccaaagaaaaaccgccagcgaaattatcagagctactcgcatagctcctccaaatccttcatgaactcaacttccggagtggtatctttccagtactgatactctttcttgagctctttggcagcctctacgagttttttggcttcatctgacgttatgtgataaatgttcataccaaccaatttatcaatatgagccttgaacaattcagttttttctagttctgttacaagctgcttacgagtttttccttgaataacaatttcacccgaaattacttgtttaatgaattgagctttagctactgccaagttcaactgagacaaaacgtcttcacgcttaaactcaattcgtttttcaacaaaagtcttacggacttcaacaaaatgtttaatgagttcagacgctttttgaaacttgtcgtttaacttgccattttcgtcaatgacaacaatgaactgcgaaagtttttcaattaatttgaaatctcgcattacagcgtcatgccgtttatcttcgtctgccggtaacccataatctttcttgaattttactttaaagccaaacccagattttgaacagtcatccgtataagtaataaaacctttgtcttctaacgggtcgagtactttttcaatataagtatcacggtcaaatttatacggaatttcactgatatacatctgtgtagctgaagtgaatttataaacaccgtgtaattcaactccaccgtcatcagtcggaacaattttaccattgaattttggaaattcaacttctggctctttatcaagctttccctgtaaagcgagtttagtgcattctacaactgatttaaaactgtgcggaagaatattagttgcgtacccggtagcaataccgcgaacaccatttaataatacggtaggaataaccggaagataaaaagctggcggtgtatgttcttcgtctttgtgttttggagcaatctctaagtctttataaactttacggaaattgtcagaaatacggcaaaagatgtaacgagacgcagcagctttttgtactaaacgcgaaccaaagttaccttgtccgtccaataaagggaaattgttattccacgtattagccatcagtgccccggcatcttgtgcagagccttcaccgtgatgataccctaaatcagctactccgccagcgactgatgcaagcttatgaaatttttctttattacctttgctgaggtctaatgctcgacgaataacaaaacgctgtactggtttaaatccatcaatcatatttggaattgcacgattttctactgtgtagattgcgaattctttagcttcagtatcaataatactttgtaaacttctttcatttaattgcatatgatttccaattaccaaatgtatgaactgacgtcaattataacacgtgtaaagaaaagcgtcatctgtagtgctataaaatataagcttgcgagaattattccaaatatcgctaaacaaatcgcggcgattgtcaaaattctcatttaattataccactataagagaataaccgctccagcgacagagcagctattccgaaacacatagcgccaagaactcttgacggaggggctaacccctcccaaataaacatcccagaaactatgaataacggtatcattataataaacacggtcgcccatatagttttaattgcattattcatattatttcctagataatttaaaattgtttaaatgcggtaatgactaaaactagaccaagagtcaattgagcaacaaactgagccacgaatgaagactctcgtgtcactgcagtgtaaattgctaaaccaaaccacaccgcggcgattaataatgtcaacatttcaaatcctcttaatatttctgcagaataaatttaaaattatcatttaacatgcgattcatttcttcgaggttctgaaatgcatcttcgtgtttacgagtgaaagctaaagacaattgtcctttaccatgacctgtcgtaagaggtttcattttttcagctggaacgaataccacgctatacacctggtgattattgctcaaagtacgaccaagttgagagcgtccttgtcgaaattgcgataaagtattagaaaacccggttttagaacgttgacgaccaacgtaactacgagataccgcaaatccagcgatattcataataaagtaaaaacctgggcgagatagttcttcttccattggaataccacccacccaatttgcgtttttaatcacgccaattacagttgcgccagcttttgacaaagtttcgatagacaaaaatgtacgacgggtcatattgatatcctcaagttaagtaatttattatcctataatatcacaaattttaattaatgtaaacggttatgatagtttccatagcaacaagctaaaacaaacgcaaatcaacgggatacataatatccaaattaagcaccaagctttactgctcatgattacctcttagtaattagtcatgttatgttttgatgaggtaatagtaacacatccttccatggatgtaaacggctgataatcaaatcattaggtaccaaccttcatacttacttcttttgacaattttcttagtgtcttctgggtccccaacgaacactggggtaaactcgaagcctccagttaaattctctacgacatctaatataattatacagtctgcaacttgatgattcaggaatggccctagattaaccccataaccatagggaaagttaccagagtatcccgtagtaactgaaatccacttagactctgattgatgtgtttttacttcaatgcggagaccacagtatcttggatgcgcaagtacatcccatgcgaaggtgtaagggtcatcatgattttcttgaccatgcattacataaccattaacccattctgcgacggccttttcagctaattgcgctatcatgcaccttttaaccgcggcctctttatcttggcctgggtcttttgacatcgtgtaatttacggtgtcttttattttaactctatcttctggagttaaatcattcttcccgcggataaatgtcggcagtttcttcagtctcaacaaccccggattgttcttgttcattttgctcacctgtataaacgtcttttacccgaagaataccacaataaattcggtcatcaaaatttaaactttctttgtttgagagagcaaattgtatgtgttctttaccaataaaaacatcatcgcaagtcagtacaccatcaacattagggtcaatcaccaaatccaaagcatacacattttgagctttgtgattgccgtgcaatcgtgattcattaattttcgaatcagaaacgttaatccaattaatcataatcatctcttaaagaaaaagggaccgaagtcccttagaattaaattagcgatgcgacggtattagaacgtgcaccatttgctagttacatcttcaattacttggtatttacatgtacgcattttagcatcgccgtaatcaaccgggatagatactacatctcgcggatgaactttaactgaaacgatacggtcactagaaccacggaaatagctaatataactcttagagcaaacatgcaatccagctgaacatgtacgttcgtcatcttcatcaacctggttacgagccatttctacagtaaccccaggggagttatcaaaagtgcggctatggcaatctttgtagttttcagtaacaactttccaagcgataaaatggccgtcatcagtgatttcaatatcgttcgcaaccaagaaatcaaacagacgagctacagctttgcgacttgggttttccatcaaattttcaaggaaaggcatatagaattcgaagttctcgccctgttccatggatttcaggatacgctctgtgagacctgatttaatttcgatgtctttatagaacagacggccttcgtcaattttaacattacctttaacgtaagaggtcaaacctttttcagcgttaacgatttccaatgcgccttcaatatcaccatcaatcaggcgctgcagagcaattttaaaaccgggtagtttatcatctgccggataagttttgtgagtgctcaaatcggtaattgagataaatttattagaagcgttccagatatattctggcgcagcaaccggagtttcaactgcttcaatttcttctactttagtttcttgagccatttctttaactacgcggcggatagtatctactgaacagttgaagtgcttagcaatccagtctcgtttgtagcctgaatcaacataatcaacgatagacaatttctgagaacgagtaaacatttgtacggccat

SEQ ID NO: 2 - DNA, 3822 bp, Unknown
  Salmonella bacteriophage S16
atggccgaattaaaacgtaagttcagagctcaagaaggtctggacgctgcgggtgagaaagtcatcaacgttgccaaggcggatcgcacggtcatgtccgatggcgttaacgttgaataccttatccaagaaaacacattacaacaatatgatagtactcgcggttatccagcacattttgctgtcatttatgataatcgtatttgggtgtctaatagagaaattgctgaaccagcaggcgattttactgagctttactggaattctgtacgcactgatgcaaaatggaaaacagtgtcatctggaacaacaaatttaaaatcaggtgattttatttcagctgacactgccggaagaacagatatgaaattcattcttcctagcaatccgcaagatggcgataccatttttattaaagatattggtggtcagccaggatatgcttctcttactgtcgatgcatctattcaatccatagtatggatgggttctcaaataagaaccgctcaaatgacgcatccatactcacaaatggttttcgttttcagtaatcgtttgtggcaactttatataagtgataacgaatcaagagccacttatataactccagctagtattcatgaagcgcaagcaggcgaaaatctagttagaagatttacttctggagcagaagtttttattactcttcctaaacatgctaacaacggtgatataatttccgtagttgatttagattctcttaacccgttgtttcatactacattaaaaacttatgaccaaaatactagtataggccaagttggaactcatgagatgcagttccgtacttccggagatggatttgtagtatttgattcagctgataatttatggagagtatgggacggtgatttacgtacccgcttgcgtattgtaactgaagatactgacgttagaccaaattctcatatcatggtttttggggctaacaatgacgaaataaagacagttaatctaaacttgcccgaatctccagctattggagacacagtaaaaatttctcttaactatatgagaaaagggcaaacagttaatataaacgctactggaactgacacaatagcttcaaatattgaacttttgcaatttccaaaacgctctgattatccgcctggagctacttgggttcaaagcagtactttaacgttcaatggtgatgaatcttatgttccaattttagatttatcttacatagaagatgaaaactctcaatattgggtagttgcagataatactccgaccgttgaaagagtcgattcgactaatgatgagacccgagctcgtttaggtgttattgctttggcaacccaagagcaagctaataaagataaagaagataatccagaaaaagaattagcgattactccagaaactttggctaaccgtattgctactaaaatccgccgcggtattgctcgtttagctactcaagctgaattagaagttaaaactggcggggctttattagatgatgtgattgttactccgaaagttctgaatgaccgtaccgctttagaagaccgtcaaggcctcgctgaattagcaactcaatcagaaactaacgatggtgttgatgattctagaattgtaactccgaaaaagttacataaccgcaaagcttcagaaatattaaccggtattttggcgttagttaaaactggaatttcaactcttgcaggtgtagatagagatacaaaaggaagtaacgtatatgactatactgataatgaaaaagctgtaactccagcttctttatttgaaaacaaagctacatatacatcacaaggtggttcatacttagctacagaaaccgaagttattcagggcactccacatgaccctaaagtgcctacagtagtaactccagtcgaattgcataagaaaactgctactgaaacacgtattggttttagtgaaattgctacgcaaaacgaagtcaatactgggacagatgatttccgttatgttactcctaagaaattaaatggccgtaatgctacggaagatttaactggtatttcgcgagtcgcgacagacgctgaattcgctgctggggagctagataatgttatttctactcctaaaaagattaagaattacttttcttctcctgaccgtaaatctgttgttactgaatctggattagttgaatcaggtaactcctgggaccattataaccttgatattcaaaaagcatctgagacgcaacgcggcacattacaattagccacacaagttctcactgacgctggggttgatgacacgactgctgtaactccgaaaaagttacaagctaaaaagacatctgaaacttctgaaggcattattcagatagcgactcagtcagaaacaacaagtggtactgtaggaaataaagctgttcctccgaagcatctaaaatatgctatccaagaacaacctgattgggaagcttctcctcttcgtagaggtccagttaaattaacagaaggcgctttaacgtttgttggtgataaagaatttggttctggtgtcaaatttgacactacaactggtctttatattaatgataatgataagttaaatgccggaaattatttcaaatctggatatgctatatctccctttgaaatgaacaagactcttcaaaactttttgccaataaacgcaactgctgttaattcgcataagttagataatcttgattcaactcagttcatcagaagagatattgaccaaacggtcgaaggttcgttaacgttaacgcaacaaacgaacactagtgctcctctagtatcctctagtactgcgaagtttgttagtatgttggttactacagaagctactatcggggacgctacgggtcactctgtgattaatttggacgctaaaaccaataaatgggttattgatggacaagctaattctcagtatttagattttactgccggaactacagatgttcttaagctcaaacgagatggagacgttaacgtagcccaaacattatcggctggtaatagagtagatgcttcaaaaggctttagcgtcgaaggtggtataatggttattaatcctaccgccaacaatattcaaattggttcacaatctaaagctactaatatccaaacaacagacgccggaaacttgaaagttactgatacatcaggctcttctgtgatattgaccactaaaaacgctgttactatcgtaggtaataacttcgttaataaagctggcgattcaatgtctggccgtttagatatttctgctgcaatgagttcagtaattactgaagctaaagcgataggtcctcttactaacgaaactgtgggtaattggtcagcagaaatcataactgaagatatctacaaaacattgcctggatttatggttccaatttattctgacgatggcggtggtaaagtaattataggttatgtagattatgacccggctgatgcatcaaaacgcggtgtaagagctccgggtgtattatctcagattggaacgagtaaaaaggaattcacatatcaaatatggaaccctcgtccagctacagcttatacagatgctaaatcttctctttggattagaacctatgacccagttaaaggtgcattcaatgaatggggaagagtttacactactgaagctcctgttacgtctgctgaaattggtgccgtttctacagctggttcttcatttaataacttaacaatcagagactggttacagattggtaatgttcgcattacgcctaatcctgatacccaatctgttgattttacttggataccttaa

SEQ ID NO: 3 - protein, 1273 aa, Unknown
  Salmonella bacteriophage S16
MAELKRKFRAQEGLDAAGEKVINVAKADRTVMSDGVNVEYLIQENTLQQYDSTRGYPAHFAVIYDNRIWVSNREIAEPAGDFTELYWNSVRTDAKWKTVSSGTTNLKSGDFISADTAGRTDMKFILPSNPQDGDTIFIKDIGGQPGYASLTVDASIQSIVWMGSQIRTAQMTHPYSQMVFVFSNRLWQLYISDNESRATYITPASIHEAQAGENLVRRFTSGAEVFITLPKHANNGDIISVVDLDSLNPLFHTTLKTYDQNTSIGQVGTHEMQFRTSGDGFVVFDSADNLWRVWDGDLRTRLRIVTEDTDVRPNSHIMVFGANNDEIKTVNLNLPESPAIGDTVKISLNYMRKGQTVNINATGTDTIASNIELLQFPKRSDYPPGATWVQSSTLTFNGDESYVPILDLSYIEDENSQYWVVADNTPTVERVDSTNDETRARLGVIALATQEQANKDKEDNPEKELAITPETLANRIATKIRRGIARLATQAELEVKTGGALLDDVIVTPKVLNDRTALEDRQGLAELATQSETNDGVDDSRIVTPKKLHNRKASEILTGILALVKTGISTLAGVDRDTKGSNVYDYTDNEKAVTPASLFENKATYTSQGGSYLATETEVIQGTPHDPKVPTVVTPVELHKKTATETRIGFSEIATQNEVNTGTDDFRYVTPKKLNGRNATEDLTGISRVATDAEFAAGELDNVISTPKKIKNYFSSPDRKSVVTESGLVESGNSWDHYNLDIQKASETQRGTLQLATQVLTDAGVDDTTAVTPKKLQAKKTSETSEGIIQIATQSETTSGTVGNKAVPPKHLKYAIQEQPDWEASPLRRGPVKLTEGALTFVGDKEFGSGVKFDTTTGLYINDNDKLNAGNYFKSGYAISPFEMNKTLQNFLPINATAVNSHKLDNLDSTQFIRRDIDQTVEGSLTLTQQTNTSAPLVSSSTAKFVSMLVTTEATIGDATGHSVINLDAKTNKWVIDGQANSQYLDFTAGTTDVLKLKRDGDVNVAQTLSAGNRVDASKGFSVEGGIMVINPTANNIQIGSQSKATNIQTTDAGNLKVTDTSGSSVILTTKNAVTIVGNNFVNKAGDSMSGRLDISAAMSSVITEAKAIGPLTNETVGNWSAEIITEDIYKTLPGFMVPIYSDDGGGKVIIGYVDYDPADASKRGVRAPGVLSQIGTSKKEFTYQIWNPRPATAYTDAKSSLWIRTYDPVKGAFNEWGRVYTTEAPVTSAEIGAVSTAGSSFNNLTIRDWLQIGNVRITPNPDTQSVDFTWIP

SEQ ID NO: 4 - DNA, 1182 bp, Unknown
  Salmonella bacteriophage S16
atggcgagttttggtaacggatataccaaaactcaagttatttctgaaaataattccatcaaatacaaaatttcatttgctgcggggtcggttttttcgaccccttcttctgcttattttacctttcaggataatccaataggtaaccagcaagatggtgctggtataaatatcagagtttttaatccagcattgaatactgtatctgctaagaaaacatttttattgacacccaatgataatgacccaggcaatagagcattcattgaatatttgtcaacatttactcaagataatactaacttgttaatattcaccacatcaggtgatattaaaacgagcaatttagtagagaataaatttaagtccatttattcaactatgtggccaaataaatggatgacatctcggtattcatgtacttattgcggtttattttcaataaaaaataataaaattattgccgaaaacgtaacatattctgatggagttcttcgggacgaagacatcagacctgctttagaattcgtttatgataaagctgatgatattggagctaccgggttttcttatagagctgttgaagattttgaagaatacactagttccgcagcaacaataaaaagatatccagttgattctgcttcgggcgtagaaattagctcaattggaatatctcctggcgatattctgttctggtcatttgaatttttgcatggagataatattccgccggaagttccagggacaaataataataaaataagaatagaaataagatggttaaattcatctggagggtggattaaatcagttaatgttgactctaaccacgcaaatgctggaaaatggatacagcacgaacaaactgttgaagtccctgctgacgctgcccggatagttattcttgcttctaaaactactccaactgatacggtcggtactggaggtgttcgtagtatgattttaactgaaacttcgcgagctactgaggcattaacttctccatctgcgatatcagttaatggtattcgtttgaatactatagtttcaggagataacccgacgctgcttattttacctgctaacgaagttgattcaactggcaaaccattaccaggtgaagatgtttcaggaataatttacagttctgattggagagagtttgagaaaaatatttaa

SEQ ID NO: 5 - protein, 393 aa, Unknown
  Salmonella bacteriophage S16
MASFGNGYTKTQVISENNSIKYKISFAAGSVFSTPSSAYFTFQDNPIGNQQDGAGINIRVFNPALNTVSAKKTFLLTPNDNDPGNRAFIEYLSTFTQDNTNLLIFTTSGDIKTSNLVENKFKSIYSTMWPNKWMTSRYSCTYCGLFSIKNNKIIAENVTYSDGVLRDEDIRPALEFVYDKADDIGATGFSYRAVEDFEEYTSSAATIKRYPVDSASGVEISSIGISPGDILFWSFEFLHGDNIPPEVPGTNNNKIRIEIRWLNSSGGWIKSVNVDSNHANAGKWIQHEQTVEVPADAARIVILASKTTPTDTVGTGGVRSMILTETSRATEALTSPSAISVNGIRLNTIVSGDNPTLLILPANEVDSTGKPLPGEDVSGIIYSSDWREFEKNI

SEQ ID NO: 6 - DNA, 666 bp, Unknown
  Salmonella bacteriophage S16
atggccgatttaaaagcaaatagtacagtaggcggagctccgatatggcataaaggaaattttcctttgtctccggtaggtgatacgctactttataagactttcaaagtctatacagaattcgataaacctcaagcagtagataacgattttgtttctaaagccgctggaggagaatatttaaaaaacgttaattttaaagaaggcttatcatttagcgacaaagatggagcttctgtttttattggtgttcctaaaaatactacagcaacagctacttataccgcatcaataaaattaaccggacaattcgctttagaaactcctgataataaaccatttatcatatttgacccaaacgagtcatttagccctgatgtatatcgcttaactgttatgggtgatatgctttctcgtcaaatttatgatgaatccggaagagttttttctcctgggaatactccgtcaaaagcgcaagttgggttagatttagtcgataacgctaagcaagttcaacttgaaaaatctggtgttcaaacaatgaccggggttttggctgctccaaactttataagtaccaatccggcaacggcagataatcatgtagctcgttttgaccagattgtaataaaagactcaattcaagattttggatattatagttaa

SEQ ID NO: 7 - protein, 221 aa, Unknown
  Salmonella bacteriophage S16
MADLKANSTVGGAPIWHKGNFPLSPVGDTLLYKTFKVYTEFDKPQAVDNDFVSKAAGGEYLKNVNFKEGLSFSDKDGASVFIGVPKNTTATATYTASIKLTGQFALETPDNKPFIIFDPNESFSPDVYRLTVMGDMLSRQIYDESGRVFSPGNTPSKAQVGLDLVDNAKQVQLEKSGVQTMTGVLAAPNFISTNPATADNHVARFDQIVIKDSIQDFGYYS

SEQ ID NO: 8 - DNA, 2250 bp, Unknown
  Salmonella bacteriophage S16
atggctactataaaacaaatacaatttaaaagaactaaagtagcaggctctcgtcctactgccgctcaactcgctgaaggcgaacttgctattaacttaaaagaccgcactattttcacgaaagacgatttgaaccaaatcattgatttaggttttgcaaaaggcggtgaagtatcaggagatatcacgcagattggtaattatacccaaactgggaactataatttaaccggcgatgctactatatcaggcaaaactactacaagcacactggatgttgggtctgtttcagatctaagacagacaaactttagaccagtattaagcacaacaactggttctaattttattatatcaaactctggcggcttgattaaaccaattactttgacggtagaaggcacagctacaagttcaaacactattctgcgccattcagttgatactactgtagcagcttctggatttatcgattcaattaatgtttctttaaatccgacggacggcgctcttgttacagctctcaatggtactgtgaatatcggtagttcccttaaaactcctaaactttcagtttctggcgcggaaactgctttgggagattatagtatttcaatcggtgataatgatacaggatttaaatggaattctgatggtgtattcagcttagttactgacagtaattcaatttatacatactctcgtgataggacatattctaaccgtccaacaaatttccggtatacgtctgactttgatgctacgacacctgctttagctccgccaggcacatggttagcttcagttgaaactgcaatagacggtaacgcttacggcgatggaatgagctatctcggttataaagataccgcaggttatagtttttatttccgcggcggcggcacttttaacgtagcttctaaaggtggatttaatgtagacacagctgcggcttttgccaaaacggttgatgtatctgatattctaacatgcagctctattattaaagctaaaggcccaggtcaagttgatgttactagtgctggtaatatcgcacttggtggaactattcaatgggtgccttcgtatatgagcggaagcccaaatagagcacgagatacaatagctacagcagcttggggcgatgccgaccaacgaattaacgtattagaaacatctgacccgcatggttggtggtattatatacagcgcgcaggttctggaagttcttctcctacgggaatagaagggcgagtcaatggctcttggcaggcatctgatttaatttctgataatactctaagagtagctggagctttcacttgtactagaagaaactccgcaggatggggtgataacgctggatggtatgctggagcaacaccagtagtagctaaccaaggaaacgttcaagaaatggaccccggtgtaggcggtttttatcctggatttgctcaatataactataatggcactggatggaaccaagcatttgttctggggttactaggccaaggtgttcagagatggcgtcgtggtgtattagctcttagaggcgatggccctgttgatgctggacaacaaattgctcgttggtattttagtcaagaagacggaagtttggaatcagaaggtccgcttaaagctcctagtgttcaagctggacaaataacatcttttggtgtaaacgttacaaacgcgttaggcagtgcgtctatagctattggcgataacgataccggattgcgctggggcggcgatggtatcgttcaaattgtagctaataacgctatcgtaggcggatggaattcaacagatatttttactgaagctggtaaacatataacatccaatggaaatttaaatcaatggggcggaggcgcaatttattgtagagaccttaatgttagttccgacagaagaattaagaaagacataaaagcatttgaaaatcccgtagatattttaagcactataggcggttatacttatcttattgaaaaaggatttaatgaagatggaagtcaggcttacgaagagtccgctggattaattgctcaagaagtagaagctgttcttcctcgtttagttaaaatatccaatgatggaacaaaagatgttaaaagacttaattataatggtataacagctttaaatactgctgctataaatgtacatactaaagaaattaatgagctcaaaaagcaattaaaagagcttaaagacattgttaagtttttaactaaataa

SEQ ID NO: 9 - protein, 749 aa, Unknown
  Salmonella bacteriophage S16
MATIKQIQFKRTKVAGSRPTAAQLAEGELAINLKDRTIFTKDDLNQIIDLGFAKGGEVSGDITQIGNYTQTGNYNLTGDATISGKTTTSTLDVGSVSDLRQTNFRPVLSTTTGSNFIISNSGGLIKPITLTVEGTATSSNTILRHSVDTTVAASGFIDSINVSLNPTDGALVTALNGTVNIGSSLKTPKLSVSGAETALGDYSISIGDNDTGFKWNSDGVFSLVTDSNSIYTYSRDRTYSNRPTNFRYTSDFDATTPALAPPGTWLASVETAIDGNAYGDGMSYLGYKDTAGYSFYFRGGGTFNVASKGGFNVDTAAAFAKTVDVSDILTCSSIIKAKGPGQVDVTSAGNIALGGTIQWVPSYMSGSPNRARDTIATAAWGDADQRINVLETSDPHGWWYYIQRAGSGSSSPTGIEGRVNGSWQASDLISDNTLRVAGAFTCTRRNSAGWGDNAGWYAGATPVVANQGNVQEMDPGVGGFYPGFAQYNYNGTGWNQAFVLGLLGQGVQRWRRGVLALRGDGPVDAGQQIARWYFSQEDGSLESEGPLKAPSVQAGQITSFGVNVTNALGSASIAIGDNDTGLRWGGDGIVQIVANNAIVGGWNSTDIFTEAGKHITSNGNLNQWGGGAIYCRDLNVSSDRRIKKDIKAFENPVDILSTIGGYTYLIEKGFNEDGSQAYEESAGLIAQEVEAVLPRLVKISNDGTKDVKRLNYNGITALNTAAINVHTKEINELKKQLKELKDIVKFLTK

SEQ ID NO: 10 - DNA, 750 bp, Unknown
  Salmonella bacteriophage S16
atggcagttcaaggaccttgggtaggttcgtcttatgtcgctgaaacaggtcaaaactgggcctcattagcggcgaatgaattaagagtaacggagaggcccttttggatttcctcatttatagggcgctctaaagaagaaatttgggaatggactggagaaaatcacagtttcaacaaagattggttaataggcgaacttcgcaatagaggtggtactcctgtagtaattaatataagagctcatcaagtgtcttatactccaggcgcacctttatttgaatttcccggagaccttccaaatgcatatattacacttaacatttatgcagatatatatggaagaggtggtactggtggagttgcttatttaggcggaaaccccggcggagactgtattcataattggattggaaacagacttagaataaacaaccaaggctggatttgtggtggcggaggcggcggcggtggctttcgtgttggacatactgaagctggtggtggtggcggacgacctttgggagcgggcggagtttcaagcttaaatctaaacggagacaatgctactttgggtgctcccgggcgaggatatcaacttggaaacgattatgcaggtaacggcggcgatgtcggtaatcctggttcagctagttcagccgaaatgggcggtggagcggctggacgagctgttgtaggaacctcacctcaatggataaatgttggtaatattgctggttcttggttataa

SEQ ID NO: 11 - protein, 249 aa, Unknown
  Salmonella bacteriophage S16
MAVQGPWVGSSYVAETGQNWASLAANELRVTERPFWISSFIGRSKEEIWEWTGENHSFNKDWLIGELRNRGGTPVVINIRAHQVSYTPGAPLFEFPGDLPNAYITLNIYADIYGRGGTGGVAYLGGNPGGDCIHNWIGNRLRINNQGWICGGGGGGGGFRVGHTEAGGGGGRPLGAGGVSSLNLNGDNATLGAPGRGYQLGNDYAGNGGDVGNPGSASSAEMGGGAAGRAVVGTSPQWINVGNIAGSWL